SEQUENCE LISTING (29 sequences)

SEQ ID NO: 1 - DNA, 49 bp, Artificial Sequence
  Synthetic - BfaI-truseqR1-pmeI-nextera7 (adapter)
  5′Phos (1)..(1)
taagatcggaagagcgtcgtgtttaaacggagatgtgtataagagacag

SEQ ID NO: 2 - DNA, 19 bp, Artificial Sequence
  Synthetic - Tn5MErev (adapter)
  5′Phos (1)..(1)
ctgtctcttatacacatct

SEQ ID NO: 3 - DNA, 20 bp, Artificial Sequence
  Synthetic - TruseqR1(splint oligo)
acacgacgctcttccgatct

SEQ ID NO: 4 - DNA, 66 bp, Artificial Sequence
  Synthetic - Nextera-pcr-i7-10-L
caagcagaagacggcatacgagatcagcctcggtctcgtgggctcggagatgtgtataagagacag

SEQ ID NO: 5 - DNA, 70 bp, Artificial Sequence
  NEB primer i501
aatgatacggcgaccaccgagatctacactatagcctacactctttccctacacgacgctcttccgatct

SEQ ID NO: 6 - DNA, 70 bp, Artificial Sequence
  Synthetic - dT30VN-ME-A
  misc_feature (34)..(37): n is a, c, g, t or u
  misc_feature (70)..(70): n is a, c, g, t or u
tcgtcggcagcgtcagatgtgtataagagacagnnnnvttttttttttttttttttttttttttttttvn

SEQ ID NO: 7 - DNA, 41 bp, Artificial Sequence
  Synthetic - NotI-TSO
  5′isodG (1)..(1)
gcggccgcaagcagtggtatcaacgcagagtacatrgrgrg

SEQ ID NO: 8 - DNA, 23 bp, Artificial Sequence
  Synthetic - IS PCR
aagcagtggtatcaacgcagagt

SEQ ID NO: 9 - DNA, 25 bp, Artificial Sequence
  Synthetic - Tn5ME-A-aHiC
agcgtcagatgtgtataagagacag

SEQ ID NO: 10 - DNA, 47 bp, Artificial Sequence
  Synthetic - Nextera i7
caagcagaagacggcatacgagatctagtacggtctcgtgggctcgg

SEQ ID NO: 11 - DNA, 51 bp, Artificial Sequence
  Synthetic - Nextera i5
aatgatacggcgaccaccgagatctacacctctctattcgtcggcagcgtc

SEQ ID NO: 12 - DNA, 25 bp, Artificial Sequence
  Synthetic - sox2-gRNA-#1-1F
caccggtgtcgtcttgtctttagtc

SEQ ID NO: 13 - DNA, 25 bp, Artificial Sequence
  Synthetic - sox2-gRNA-#1-1R
aaacgactaaagacaagacgacacc

SEQ ID NO: 14 - DNA, 25 bp, Artificial Sequence
  Synthetic - sox2-gRNA-#1-2F
caccgggaccataggttcctagagc

SEQ ID NO: 15 - DNA, 25 bp, Artificial Sequence
  Synthetic - sox2-gRNA-#1-2R
aaacgctctaggaacctatggtccc

SEQ ID NO: 16 - DNA, 25 bp, Artificial Sequence
  Synthetic - sox2-gRNA-#1-3F
caccggggcagccttgatgtcctaa

SEQ ID NO: 17 - DNA, 25 bp, Artificial Sequence
  Synthetic - sox2-gRNA-#1-3R
aaacttaggacatcaaggctgcccc

SEQ ID NO: 18 - DNA, 25 bp, Artificial Sequence
  Synthetic - sox2-gRNA-#2-1F
caccgtccccgtgcattgaagaaag

SEQ ID NO: 19 - DNA, 25 bp, Artificial Sequence
  Synthetic - sox2-gRNA-#2-1R
aaacctttcttcaatgcacggggac

SEQ ID NO: 20 - DNA, 25 bp, Artificial Sequence
  Synthetic - sox2-gRNA-#2-2F
caccgcaggtgtcttgcctgcccta

SEQ ID NO: 21 - DNA, 25 bp, Artificial Sequence
  Synthetic - sox2-gRNA-#2-2R
aaactagggcaggcaagacacctgc

SEQ ID NO: 22 - DNA, 25 bp, Artificial Sequence
  Synthetic - sox2-gRNA-#2-3F
caccggcagcagaaggttctttagc

SEQ ID NO: 23 - DNA, 25 bp, Artificial Sequence
  Synthetic - sox2-gRNA-#2-3R
aaacgctaaagaaccttctgctgcc

SEQ ID NO: 24 - DNA, 25 bp, Artificial Sequence
  Synthetic - sox2-gRNA-#3-1 F
caccgaaagcgagcgccctgattaa

SEQ ID NO: 25 - DNA, 25 bp, Artificial Sequence
  Synthetic - sox2-gRNA-#3-1 R
aaacttaatcagggcgctcgctttc

SEQ ID NO: 26 - DNA, 25 bp, Artificial Sequence
  Synthetic - sox2-gRNA-#3-2 F
caccgtcccgggagtaacgagcaag

SEQ ID NO: 27 - DNA, 25 bp, Artificial Sequence
  Synthetic - sox2-gRNA-#3-2 R
aaaccttgctcgttactcccgggac

SEQ ID NO: 28 - DNA, 25 bp, Artificial Sequence
  Synthetic - sox2-gRNA-#3-3 F
caccggttactcccgggagaggcgc

SEQ ID NO: 29 - DNA, 25 bp, Artificial Sequence
  Synthetic - sox2-gRNA-#3-3 R
aaacgcgcctctcccgggagtaacc

(19) United States
(12) Patent Application Publication
(10) Pub. No.: US 2024/0052338 A1
(43) Pub. Date: Feb. 15, 2024

(54) COMPOSITIONS FOR AND METHODS OF CO-ANALYZING CHROMATIN STRUCTURE AND FUNCTION ALONG WITH TRANSCRIPTION OUTPUT

(71) Applicant: Duke University, Durham, NC (US)

(72) Inventors: Yarui Diao, Durham, NC (US); Xiaolin Wei, Durham, NC (US); Yu Xiang, Durham, NC (US)

(73) Assignee: Duke University, Durham, NC (US)

(21) Appl. No.: 18/033,002

(22) PCT Filed: Nov. 2, 2021
(86) PCT No.: PCT/US2021/057742

§ 371 Date: Apr. 20, 2023

Related U.S. Application Data
(60) Provisional application No. 63/108,565, filed on Nov. 2, 2020.

(51) Int. Cl. C12N 15/10; G16B 45/00

(52) U.S. Cl. CPC C12N 15/1065; G16B 45/00

(57) ABSTRACT

Disclosed herein are compositions for and methods of performing a multi-omics assay comprising analyzing chromatin structure and function and analyzing the transcriptome using the same population of cells. Disclosed herein are compositions for and methods of performing a high-throughput chromosome conformation capture on accessible DNA and mRNA-Seq co-assay (HiCAR).

### II. CROSS-REFERENCE TO RELATED APPLICATIONS

This application claims the benefit of priority to U.S. Provisional Patent Application No. 63/108,565 filed 2 Nov. 2020, the entirety of which is incorporated by reference herein.

### I. STATEMENT REGARDING FEDERALLY SPONSORED RESEARCH OR DEVELOPMENT

This invention was made with government support under Grant No. U01HL156064 awarded by National Institute Health (NIH). The government has certain rights in the invention.

### III. REFERENCE TO THE SEQUENCE LISTING

The Sequence Listing submitted 2 Nov. 2021 as a text file named “21_2028_WO_Sequence_Listing”, created on 2 Nov. 2021 and having a size of 7 kilobytes is hereby incorporated by reference pursuant to 37 C.F.R. § 1.52(e)(5).

### IV. BACKGROUND

Cis-regulatory elements (cREs), such as enhancers, promoters, insulators and silencers, play a critical role in regulating spatial-temporal gene expression in development and diseases (Gerstein M B, et al. (2012) Nature. 489:91-100; Roadmap Epigenomics Consortium. et al. (2015) Nature. 518:317-330 (2015): Diao Y, et al. (2017) Nat. Methods. 14:629-635). CREs are characterized by the presence of “open” or accessible chromatin that is depleted of packaging nucleosome particles, making way for the binding of Transcription Factors (TFs) and a variety of epigenetic remodelers. These accessible chromatin regions can be identified by Assay for Transposase-Accessible Chromatin using sequencing (ATAC-Seq), DNase-Seq, and FAIRE-Seq (Formaldehyde-Assisted Isolation of Regulatory Elements). cREs can form dynamic high-order chromatin interactions to precisely control the expression of distal target genes.

The development of chromosome conformation capture (3C)-based technologies has greatly improved the understanding of the principles of high-order chromatin organization and revealed how dynamic chromatin looping affects gene expression in a cell type specific manner. Among these technologies, Hi-C has been widely used to measure genome-wide chromatin architecture (Lieberman-Aiden E, et al. (2009) Science. 326:289-293: Dixon J R, et al. (2012) Nature. 485:376-380) but requires extremely deep sequencing depth (e.g., several billions of reads) to resolve chromatin interactions at 5 KB to 10 KB resolution. To reduce the sequencing costs, alternative methods such as ChIA-PET, HiChiP, PLAC-seq, and Capture-C have been developed. However, these methods rely on ChIP-grade antibody (ChIA-PET, HiChIP and PLAC-seq) or pre-designed capture probes (Capture-C) to enrich a subset of chromatin interactions associated with specific proteins, histone modifications, or targeted genome regions. More recently, Trac-looping and Ocean-C have been developed to analyze interactions among accessible chromatin regions, independent of ChIP antibodies or capture probes (Lai B, et al. (2018) Nat. Methods. 15:741-747; Li T, et al. (2018) Genome Biol. 19:54). Although these two methods do not require targeted immunoprecipitation or DNA pulldown, the methods require a large number of cells and yield a relatively low proportion of long-range cis reads. This prevents their application to low input materials (e.g., clinical samples and primary tissues). Moreover, none of the methods described above enable the simultaneous assessment of the transcriptome from the same biological sample, which is the key functional output of genome architecture and chromatin accessibility.

Therefore, a robust. sensitive, and cost-effective method is urgently needed to enable a comprehensive co-analysis of chromatin structure and function as well as transcription output using low-volume materials.

### V. BRIEF DESCRIPTION OF THE FIGURES

FIG. 1A-FIG. 1E provides an overview of HiCAR experimental design and HiCAR data quality control. FIG. 1A is a schematic identifying the steps of a HiCAR experiment. The nuclei were isolated from cross-linked cells and treated by Tn5 transposase loaded with engineered DNA adaptors, followed by restriction enzyme digestion with 4 base cutter CviQI and in situ ligation. The engineered Tn5 adaptors were ligated to the proximal genomic DNA digested by CviQI. After in situ ligation, the genomic DNA were purified after reverse crosslinking, and subjected to a second restriction enzyme digestion by another 4-base cutter NlaIII. Then, the resulting DNA fragments were circularized and PCR amplified for deep sequencing. The DNA sequences amplified from the splint oligo sequence and the Tn5/ME region were defined as R1 reads and R2 reads, respectively. The cytoplasmic and nucleic RNA fractions were collected and pooled together for RNA-Seq analysis. FIG. 1B shows the aggregated signals of HiCAR R2 reads (red), R1 reads (blue), and in situ Hi-C (black) within +/−3 KB window centered at H1 hESC ATAC-seq peaks. The HiCAR R1, R2, and Hi-C reads were normalized against sequence depten (counts per million). Signal coverage (y-axis) was calculated as sequencing read depth per base within +/−2 KB window of peak center. FIG. 1C shows the aggregated signals of HiiCAR R2 reads (red), Trac-looping reads (green), Ocean-C reads (orange), and in situ Hi-C reads (blue) within +/−2 KB window centered at TSS. Enrichment was calculated by comparing the normalized reads signal on peak center against the signal at +/−2 KB region. FIG. 1D shows the number of input cells and sequencing outputs of three methods. FIG. 1E shows the percentage of uniquely mapped short range (<20 KB) cis, long range (>20 KB) cis, and the trans (inter-chromosomal) reads from HiCAR, in situ Hi-C, and Trac-looping data. FIG. 1F shows the contact frequency as a function of distance measured by HiCAR, in situ Hi-C, and Trac-looping data.

FIG. 2A-FIG. 2H demonstrate that HiCAR captures the key features of chromatin organization, chromatin accessibility, and transcriptome. FIG. 2A shows the contact matrices of H1 hESC obtained from HiCAR (top right, above the diagonal) and in situ Hi-C (bottom left below the diagonal) data at successive zoom-in views. The H1 hESC in situ Hi-C data was obtained from 4DN data portal. The color represents sequence depth normalized reads signal (counts per million mapped reads). FIG. 2B is a series of scatter plots showing the global correlation of compartment scores (left panel), TAD insulation score (middle panel) and TAD directionality index (right panel) computed from HiCAR and in situ Hi-C. respectively. The R value: Pearson correlation coefficient. FIG. 2C shows aggregated HiCAR (top row) and in situ Hi-C (bottom row) contact matrix (10 KB bin) within +/−250 KB window centered on the indicated peak regions of Hi hESC. FIG. 2D is a representative genome browser view showing the signals of HiCAR RNA-Seq (pink) and HiCAR 1D open chromatin profile (light blue). The red track indicates the H1 hESC bulk RNA-Seq and the dark blue track indicates ATAC data, downloaded from ENCODE and 4DN data portal, respectively. FIG. 2E is a scatter plot showing the correlation of HiCAR RNA-Seq vs. bulk RNA-Seq dataset. FIG. 2F is a scatter plot showing the correction of HiCAR R2 reads compared to ATAC-seq reads. FIG. 2G is a Venn diagram showing open chromatin peaks identified by RiCAR R2 reads (ID open chromatin peaks) and ATAC-Seq in H1 hESC. MACS2 was used for peak calling. FIG. 2H compared the open chromatin peaks identified by HiCAR R2 reads and ATAC-seq. The overlapping open chromatin peaks and the non-overlapping peaks are separated. Boxplot showing the distribution of the MACS p value of the peaks. Wilcoxon rank-sum test was used for statistical analysis to compute p value.

FIG. 3A-FIG. 3F identifies long-range cis-regulatory chromatin interactions with HiCAR. FIG. 3A is a genome browser screenshot showing ChIP-seq (NANOG, SOX2, CTCF, H3K4mel, H3K4me3), RNA-Seq, ATAC-seq of H1 hESC, as well as the chromatin loops and interactions identified by HiCAR. CTCF HiChIP, H3K4me3 PLAC-seq and in situ Hi-C data with H1 or 119 hESCs. FIG. 3B defines chromatin loops and interactions with at least one anchor overlapping with ATAC-seq peaks as “testable” loops/interactions. The proportion of the “testable” loops/interactions that can be discovered by HiCAR interaction was calculated to estimate the sensitivity of HiCAR interaction calling. FIG. 3C shows the orientation of CTCF motif located on the pairwise anchors of each chromatin loop and interactions. The length of the color bar indicates the proportion of convergent, tandem, and divergent CTCF motif pairs among tested HiCCUPS loops and MAPS interactions. FIG. 3D shows that the TSS-eQTL pairs identified in human pluripotent stem cells were significantly enriched on HiCAR interactions. Red line represents the number of observed eQTL-TSS pairs overlapping with HiCAR interactions. The histogram represents the distribution of the number of eQTL-TSS pairs overlapped with randomly sampled (10,000 times shuffling) pairwise DNA regions with matched linear genomic distance to HiCAR interactions. (Empirical p-value <0.0001). FIG. 3E is a genome browser screenshot showing H1 hESC ATAC-seq track and HiCAR interactions near SOX2 locus. The three arrowheads point to the three candidate SOX2 enhancers (highlighted in light blue).

FIG. 3F shows the mRNA expression of SOX2 after the Hi hESC were infected by lentiviral vectors expressing dCas9-KRAB together with control sgRNA or the sgRNAs targeting enhancer regions. The sgRNAs were designed to specifically target the SOX2 candidate enhancers showing in FIG. 3E. After lentiviral infection, the hESCs were selected by puromycin for 3-days, then cultured for another 7-days without puromycin. The total RNA was extracted and subjected to RT-qPCR analysis. The mRNA level of SOX2 was normalized against housekeeping gene GAPDH. The data was collected from three biological replicates. P values were calculated by two-tailed Student's t test.

FIG. 4A-FIG. 4E demonstrate that the poised. bivalent, and repressed chromatin regions form massive, long-range, and significant chromatin interactions comparable to the active chromatin states. FIG. 4A shows thee fold change (y-axis) of HiCAR interaction for each chromHMM state, which was calculated as “observed/expected”. The fold change of Hi-C loops for each chromHMM state was calculated in the same way. The anchor (5 KB bin) sequences of all interactions identified by HiCAR were used and the “observed” number of anchors overlapped with each individual chromatin state defined by chromHMM were calculated. Based on the genome-wide distribution of each chromHMM state, the “expected” number of anchors overlapped with each state was also calculated. FIG. 4B shows the “observed” interaction frequency of pairwise chromatin states (total 18 states determined by ChromHMM) based on HiCAR interaction. Based on the genome-wide distribution of each chromHMM state, the “expected” interaction frequency between any two states was calculated. The fold change of pairwise interaction frequency and P-value were calculated using the “annotateInteractions” function from Homer. X-axis: log 2 (fold change) of “observed” interaction frequency over “expected” interaction frequency. Y-axis: −log 10(FDR), the FDR is the output from HOMER. Red dots: the interactions between “active” chromatin states; Blue dots: the interactions between “inactive” states, including bivalent/repressed/poised chromatin states; Purple dots: the interactions between “active” versus “inactive” states. FIG. 4C shows the mRNA level of genes expressed from the promoters located on anchors for 14,845 and 10,287 HiCAR interactions with at least one anchor overlapped with H3K37ac and H3K27me3 peaks, respectively. FIG. 4D shows the interaction strength quantified by −log 10 FDR (where the FDR is output from MAPS) for 14,845 and 10.287 HiCAR interactions with at least one anchor overlapped with H3K37ac and 3K27me3 peaks, respectively. FIG. 4E shows the linear genomic distance between anchors of interactions. The P value for the boxplot is calculated from Wilcoxon rank-sum test.

FIG. 5A-FIG. 5C identifies those epigenome features important for chromatin spatial interactive activity. FIG. 5A represents the 5 KB anchors of HiCAR interactions ranked along the x-axis based on their cumulative interactive score (sum of −log 10 FDR, y-axis). FDR is the output of MAPS of each significant interaction. Total 2,096 anchors were identified as interaction hotspots associated with abnormal high-level interactive score (red dots. described infra). FIG. 5B is a scatterplot showing the significantly enriched (red dots) or depleted (blue dot, ZNF274) histone mark and TF binding on interaction hotspots versus regular interaction anchors. For signal enrichment analysis, the 75 public ChIP-seq data listed in Table 1 was used. FIG. 5C presents the results from employing five machine learning algorithms (including Decision tree, Linear regression, XGBoost, Random forest, and Linear-kernel support vector machine) to predict the top ranked epigenome features that are potentially important for the spatial interactive activity of cREs. The “union features” were defined as the features predicted by at least two algorithms. The features highlighted in blue color were the features with known function in regulating 3D chromatin interactions.

FIG. 6A-FIG. 6E show the HiCAR library enrichment analysis and data quality control. FIG. 6A provides the aggregated signals of HiCAR R2 reads (red), R1 reads (blue), and in situ Hi-C (black) reads within +/−3 KB window of indicated peak regions of H1 hESC. The HiCAR R1, R2, and Hi-C reads were normalized against sequence depth (counts per million). Signal coverage (y-axis) was calculated as sequencing read depth per base within +/−2 KB window of peak center. FIG. 6B provides the aggregated signals of HiCAR R2 reads (red). R1 reads (blue), H3K4mel HiChIP (purple), H3K4me3 PLAC-seq (black), and DNase Hi-C (brown) within +/−2 KB window centered at TSS. Enrichment fold was calculated by comparing the reads coverage on peak center against the reads coverage at +/−2 KB region. FIG. 6C shows the use of HiCrep to compute the similarity of chromatin contact matrice including three HiCAR biological replicates and 4DN in situ Hi-C data. The number was the SCC value computed from HiCrep. FIG. 6D provides scatter plots with PCC of the reads counts from two biological replicates of HiCAR RNA-Seq library (left) and HiCAR DNA library R2 reads (right panel). FIG. 6E shows the HiCAR 1D open chromatin peaks are called by MACS2. The peaks were ranked along x-axis based on their MACS p value (−log 10). At a given P value, the y-axis indicated the proportion of the HiCAR 1D peaks that could be validated by H1 hESC ATAC-seq peaks.

FIG. 7A-FIG. 7B show the gene ontology terms associated with H3K27ac- and H3K27m3-anchored HiCAR interactions, respectively. Those genes whose promoters overlapped with HiCAR interaction anchors were selected for gene ontology (GO) enrichment analysis. FIG. 7A shows GO terms enriched on 1H3K27ac-anchored interactions while FIG. 7B shows GO terms enriched on H3K27me3-anchored interactions.

FIG. 8A-FIG. 8E show the spatial interactive activity of cis-regulatory sequence had a very weak correlation with its transcriptional activity, enhancer activity, or chromatin accessibility. FIG. 8A-FIG. 8C are scatter plots showing the cumulative interactive score (sum of −log 10FDR) of HiCAR interaction anchor on y-axis, against x-axis showing the mRNA level (log 2 FPKM) of the genes expressed from the promoters overlapped with anchors (FIG. 5A), H3K27ac ChIP-seq signal of anchors indicating their enhancer activity mark (FIG. 8B), and chromatin accessibility of anchors measured by ATAC-seq signal (FIG. 8C). PCC means Pearson correlation coefficient. FIG. 5D is a histogram showing the distribution of mRNA levels expressed from the gene promoters overlap with HiCAR interaction hotspots or regular anchors. FIG. 8E is boxplot showing the distribution of mRNA levels expressed from the gene promoters that overlapped with HiCAR interaction hotspots or regular anchors. The p value (0.96) was calculated by Wilcoxon rank-sum test in FIG. 5D.

FIG. 9A-FIG. 9B demonstrate the use of machine learning to predict histone mark and TF binding important for cRE's spatial interactive activity. FIG. 9A shows the top ranked 15 features predicted by five machine learning algorithms (i.e., Decision tree, Linear regression, XGBoost. Random forest, and Linear-kernel support vector machine (Linear SVM)). FIG. 9B shows mean absolute error and Mean squared error of each regression method.

FIG. 10A-FIG. 10F identify long-range cis-regulatory chromatin interaction in GM12878 and mESCs with HiCAR. FIG. 10A is a genome browser screenshot showing CTCF ChIP-Seq. DNase hypersensitive (DH4S), and the HiCCUPS loops and MAPS interactions identified by HiCAR. in situ Hi-C, and SMC1A HiChIP in GM12878 cells. FIG. 10B is a genome browser screenshot showing H3K27ac ChIP-seq and the HiCCUPS loops and MAPS interactions identified by HiCAR. in situ Hi-C, CTCF PLAC-seq, and H3K4me3 PLAC-seq in mESC cells. FIG. 10C-FIG. 10D describe the chromatin loops and interactions with at least one anchor overlapping with ATAC-seq peaks, which are defined as “testable” loops/interactions. The proportion of the “testable” loops/interactions that could be discovered by HiCAR interaction was calculated to estimate the sensitivity of HiCAR interaction calling in GM12878 and mESCs. FIG. 10C shows that in GM12878 cells, HiCAR discovered 79% and 62% of “testable” loops/interactions identified by in situ Hi-C and SMC1A HiChIP, respectively. FIG. 10D shows that in mESC, HiCAR discovered 74%, 70%, and 85% of “testable” loops and interactions identified by in situ Hi-C, H3K4me3 PLAC-seq, and CTCF PLAC-seq, respectively. FIG. 10E-FIG. 10F show the examination of the motif orientation of CTCF on the anchors of chromatin loop and interactions. The length of the bars indicated the proportion of chromatin loops/interactions that harbored convergent, tandem, and divergent CTCF motif on their anchors. FIG. 10E show that in GM12878 cells, 72.4%, 75.8%, and 89.8% HiCAR interactions, SMC1A HiChIP interactions, and in situ Hi-C loops harbored convergent CTCF motif on their anchors. FIG. 10F shows that in mESC cells, 63.7%, 62.7%, and 55.7% of HiCAR interactions, CTCF PLAC-seq interactions, and H3K4me3 PLAC-seq interactions harbored convergent CTCF motif on their anchors.

FIG. 11 shows the HiCAR data processing pipeline.

### VI. BRIEF SUMMARY

Disclosed herein is a method of performing a multi-omics assay, the method comprising analyzing chromatin structure and function; and analyzing the transcriptome, wherein the steps are performed using the same population of cells.

Disclosed herein is a method of performing a multi-omics assay, the method comprising using a population of cells to generate DNA for analyzing chromatin structure and function; and using the same population of cells to generate RNA for analyzing the transcriptome, wherein the method identifies cis-regulatory chromatin interactions, characterizes chromatin accessibility, and analyzes the transcriptome in a population of cells.

Disclosed herein is a method of performing a multi-omics assay, the method comprising identifying cis-regulatory chromatin interactions; characterizing chromatin accessibility; and analyzing the transcriptome, wherein the steps are performed using the same population of cells.

Disclosed herein is a method of performing a multi-omics assay in a single population of cells, the method comprising (i) identifying cis-regulatory chromatin interactions and characterizing chromatin accessibility by purifying and tagmenting DNA and performing PCR using the purified and tagmented DNA; and (ii) analyzing the transcriptome by collecting cytoplasmic and nucleic RNA while performing step (i) and creating an RNA-Seq library using the collected RNA.

Disclosed herein are methods of performing a multi-omics assay comprising (i) identifying chromatin interactions and assessing chromatin accessibility, wherein identifying chromatin interactions and assessing chromatin accessibility comprises incubating isolated nuclei with an assembled Tn5 transposomes; digesting the isolated nuclei with a restriction enzyme; incubating the digested nuclei with a splint oligonucleotide; ligating in situ the Tn5 adaptor to the proximal genomic DNA; reversing the crosslink; purifying the reverse cross-linked DNA and dissolving the purified DNA; digesting the purified DNA with a restriction enzyme; circularizing the digested DNA and purifying the circularized DNA; digesting the purified DNA with a restriction enzyme; performing PCR to generate DNA libraries; and (ii) sequencing RNA, wherein sequencing RNA comprises collecting supernatant comprising cytoplasmic RNA; collecting supernatant comprising the nucleic RNA; combining the supernatant comprising cytoplasmic RNA and the supernatant comprising nucleic RNA and reversing the crosslink; purifying the reverse crosslinked RNA, dissolving the purified RNA, and treating the purified RNA with DNase to remove DNA in solution; and using the purified RNA to create an RNA-Seq library.

Disclosed herein is a method of performing a high-throughput chromosome conformation capture on accessible DNA and mRNA-Seq co-assay (HiCAR), the method comprising incubating isolated nuclei with an assembled Tn5 transposomes; digesting the isolated nuclei with a first restriction enzyme; incubating the digested nuclei with a splint oligonucleotide; ligating in situ the Tn5 adaptor to the proximal genomic DNA; reversing the crosslink; purifying the reverse cross-linked DNA and dissolving the purified DNA; digesting the purified DNA with a second restriction enzyme; circularizing the digested DNA and purifying the circularized DNA; digesting the purified DNA with a third restriction enzyme; performing PCR to generate DNA libraries; and creating a RNA-Seq library, wherein the method identifies cis-regulatory chromatin interactions. characterizes chromatin accessibility, and analyzes the transcriptome in the population of cells.

Disclosed is a method of performing a high-throughput chromosome conformation capture on accessible DNA and mRNA-Seq co-assay (HiCAR), the method comprising isolating nuclei from a population of cells; incubating the isolated nuclei with an assembled Tn5 transposomes; digesting the isolated nuclei with a first restriction enzyme; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with a second restriction enzyme; circularizing the digested DNA and purifying the circularized DNA; digesting the purified DNA with a third restriction enzyme; performing PCR to generate DNA libraries; and creating a RNA-Seq library, wherein the method identifies cis-regulatory chromatin interactions, characterizes chromatin accessibility, and analyzes the transcriptome in the population of cells.

Disclosed is a method of performing a high-throughput chromosome conformation capture on accessible DNA and mRNA-Seq co-assay (HiCAR), the method comprising isolating nuclei from a population of cells; incubating the isolated nuclei with an assembled Tn5 transposome; digesting the isolated nuclei with CviQI; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with NIaIII; circularizing the digested DNA and purifying the circularized DNA: digesting the purified DNA with PmeI; performing PCR to generate DNA libraries; and creating an RNA-Seq library, wherein the method identifies cis-regulatory chromatin interactions, characterizes chromatin accessibility, and analyzes the transcriptome in the population of cells.

Disclosed is a method of performing a high-throughput chromosome conformation capture on accessible DNA and mRNA-Seq co-assay (HiCAR), the method comprising incubating the isolated nuclei with an assembled Tn5 transposome: digesting the isolated nuclei with CviQI; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with NIaIII; circularizing the digested DNA and purifying the circularized DNA; digesting the purified DNA with PmeI; performing PCR to generate DNA libraries; and creating an RNA-Seq library wherein the method identifies cis-regulatory chromatin interactions, characterizes chromatin accessibility, and analyzes the transcriptome in the population of cells.

Disclosed herein is a method of performing a genome-wide profiling of chromatin interactions and/or accessibility and gene expression, the method comprising performing PCR using purified and tagmented DNA; and creating an RNA-Seq library using cytoplasmic and nucleic RNA, wherein the steps are performed using the same population of cells.

Disclosed herein is a method of performing a co-assay, the method comprising (i) purifying and tagmenting DNA; (ii) performing PCR using the DNA of step (i); (iii) collecting cytoplasmic and nucleic RNA during step (i); and (iv) creating an RNA-Seq library using the RNA of step (iii), wherein the method identifies cis-regulatory chromatin interactions, characterizes chromatin accessibility, and analyzes the transcriptome in a population of cells.

Disclosed herein is a kit comprising one or more components and/or reagents for use in a disclosed method of performing a multi-omics assay. Disclosed herein is a kit comprising one or more components and/or reagents for use in a disclosed method of performing a high-throughput chromosome conformation capture on accessible DNA and mRNA-Seq co-assay (HiCAR). Disclosed herein is a kit comprising one or more components and/or reagents for use in a disclosed method of genome-wide profiling of chromatin interactions and/or accessibility and gene expression. Disclosed herein is a kit comprising one or more components and/or reagents for use in a disclosed method of performing a co-assay. Disclosed herein is a kit comprising one or more components and/or reagents for use in a disclosed method of identifying chromatin interactions and assessing chromatin accessibility. Disclosed herein is a kit comprising one or more components and/or reagents for use in a disclosed method of sequencing RNA.

### VII. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Relevant Definitions

Before the present compositions and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, a “subject” can be a source of a population of cells used in a disclosed method. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a disease or disorder, be suspected of having a disease or disorder, or be at risk of developing and/or acquiring a disease or disorder (such as, for example, a disease or disorder having chromatin deregulation and/or chromatin dysregulation). In an aspect, a subject can be diagnosed with or can be suspected of having a critical limb ischemia (CLI).

As used herein, the term “diagnosed” means having been subjected to an examination by a person of skill, for example, a physician, and found to have a condition that can be diagnosed or treated by one or more of the disclosed compositions or by one or more of the disclosed methods. For example, “diagnosed with a disease or disorder” means having been subjected to an examination by a person of skill, for example, a physician, and found to have a condition that can be treated by one or more of the disclosed compositions or by one or more of the disclosed methods. For example, “suspected of having a disease or disorder” can mean having been subjected to an examination by a person of skill, for example, a physician, and found to have a condition that can likely be treated by one or more of the disclosed compositions or by one or more of the disclosed methods. In an aspect, an examination can be physical, can involve various tests (e.g., blood tests, genotyping, biopsies, etc.) and assays (e.g., enzymatic assay), or a combination thereof.

As used herein, “fragmenting” or “digesting” nucleic acids (e.g., chromatin) can employ the use of restriction enzymes. As known to the art, a restriction enzyme can have a restriction site of 1, 2, 3, 4, 5, or 6 bases long. Following restriction, the resulting fragments can vary in size.

As used herein, an adapter oligonucleotide can include any oligonucleotide having a sequence, at least a portion of which is known, that can be joined to a target polynucleotide. Adapter oligonucleotides can comprise DNA. RNA, nucleotide analogues, non-canonical nucleotides, labeled nucleotides, modified nucleotides, or combinations thereof. Adapter oligonucleotides can be single-stranded, double-stranded, or partial duplex. In general, a partial-duplex adapter comprises one or more single-stranded regions and one or more double-stranded regions. Different adapters can be joined to target polynucleotides in sequential reactions or simultaneously. For example, the first and second adapters can be added to the same reaction. Adapters can be manipulated prior to combining with target polynucleotides. For example, terminal phosphates can be added or removed (such as, for example, with SEQ ID NO:01 and SEQ ID NO:02).

Adapter oligonucleotides can have any suitable length, at least sufficient to accommodate the one or more sequence elements of which they are comprised. Adapters can be about, less than about, or more than about 10, 15, 20, 25, 30, 35, 40, 45, 50, 55, 60, 65, 70, 75, 80, 90, 100, 200, or more nucleotides in length. Adaptors can be about 10 to about 50 nucleotides in length, or about 20 to about 40 nucleotides in length.

As used herein, “inhibit.” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a disease or disorder having chromatin deregulation and/or chromatin dysregulation). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease. pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease. pathological condition, or disorder (such as a disease or disorder having chromatin deregulation and/or chromatin dysregulation). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established disease or disorder in a subject by 1%-100% as compared to a control (such as, for example, an individual not having a disease or disorder having chromatin deregulation and/or chromatin dysregulation). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder having chromatin deregulation and/or chromatin dysregulation. For example, treating a disease or disorder having chromatin deregulation and/or chromatin dysregulation can reduce one or more symptoms in a subject by 1%-100% as compared to a control (such as, for example, an individual not having a disease or disorder having chromatin deregulation and/or chromatin dysregulation). In an aspect, treating can refer to 1%, 2%, 3%, 4%, 5%, 6%, 7%, 8%, 9%, 10%, 20%, 30%, 40%. 50%, 60%, 70%, 80%, 90%, 100% reduction of one or more symptoms of an established disease or disorder having chromatin deregulation and/or chromatin dysregulation. It is understood that treatment does not necessarily refer to a cure or complete ablation or eradication of a disease or disorder having chromatin deregulation and/or chromatin dysregulation. However, in an aspect, treatment can refer to a cure or complete ablation or eradication of a disease or disorder having chromatin deregulation and/or chromatin dysregulation. In an aspect, a disease or disorder can be critical limb ischemia (CLI).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent” and “preventing” and “prevention” also refer to prophylactic or preventative measures for protecting or precluding a subject (e.g., an individual) not having a given a disease or disorder associated with chromatin deregulation and/or chromatin dysregulation or related complication from progressing to that complication. In an aspect, a disease or disorder can be critical limb ischemia (CLI).

By “determining the amount” is meant both an absolute quantification of a particular analyte (e.g., an mRNA sequence containing a particular tag) or a determination of the relative abundance of a particular analyte (e.g., an amount as compared to a mRNA sequence including a different tag). The phrase includes both direct or indirect measurements of abundance (e.g., individual mRNA transcripts may be quantified or the amount of amplification of an mRNA sequence under certain conditions for a certain period of time may be used a surrogate for individual transcript quantification) or both.

As used herein, “fixative” or “cross-linker” can generally refer to an agent that can fix or cross-link cells. As known to the art, fixing or cross-linking cells can stabilize protein-nucleic acid complexes in the cell.

As used herein, “multi-omics” provides clinicians and researchers an opportunity to understand that flow of information that underlies various disease and disorders. Multi-omics includes but is not limited to “genomics”, “epigenomics”, “transcriptomics”, “proteomics”, “metabolomics”, and “microbiomics”.

As used herein, “modifying the method” can comprise modifying or changing one or more features or aspects of one or more steps of a disclosed method. For example, in an aspect, a method can be altered by changing the amount of one or more of the disclosed components and/or reagents, or by changing the frequency of administration of one or more of the components and/or reagents, or by changing the duration of time one or more of the disclosed components and/or reagents are administered to a subject, or by substituting for one or more of the disclosed components and/or reagents with a similar or equivalent component and/or reagent.

As used herein, “concurrently” means (1) simultaneously in time, or (2) at different times during the course of a common schedule.

The term “contacting” as used herein refers to bringing one or more of the disclosed components and/or reagents to a target area or intended target area in such a manner that the one or more of disclosed components and/or reagents exert an effect on the intended target or targeted area either directly or indirectly.

In an aspect, “determining” can also refer to measuring or ascertaining the level of one or more RNAs in a biosample or population of cells or measuring or ascertaining the level or one or more RNAs or miRNAs in a biosample or population of cells. Methods and techniques for determining the level of RNAs are known to the art and are disclosed herein. In an aspect, “determining” can also refer to identifying and/or characterizing chromatin interactions and/or chromatin accessibility in one or more populations of cells.

As used herein, the term “package insert” is used to refer to instructions customarily included in commercial packages of therapeutic products, that contain information about the indications, usage, dosage, administration, contraindications and/or warnings concerning the use of such therapeutic products.

Disclosed are the components to be used to prepare the disclosed components and/or reagents as well the disclosed components and/or reagents used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-f), C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspects or combination of aspects of the disclosed methods.

### B. Methods of Performing a Multi-Omics Assay

Disclosed herein is a method of performing a multi-omics assay, the method comprising analyzing chromatin structure and function; and analyzing the transcriptome, wherein the steps are performed using the same population of cells.

Disclosed herein is a method of performing a multi-omics assay, the method comprising using a population of cells to generate DNA for analyzing chromatin structure and function; and using the same population of cells to generate RNA for analyzing the transcriptome, wherein the method identifies cis-regulatory chromatin interactions, characterizes chromatin accessibility, and analyzes the transcriptome in a population of cells.

Disclosed herein is a method of performing a multi-omics assay, the method comprising identifying cis-regulatory chromatin interactions; characterizing chromatin accessibility; and analyzing the transcriptome, wherein the steps are performed using the same population of cells.

Disclosed herein is a method of performing a multi-omics assay in a single population of cells, the method comprising (i) identifying cis-regulatory chromatin interactions and characterizing chromatin accessibility by purifying and tagmenting DNA and performing PCR using the purified and tagmented DNA; and (ii) analyzing the transcriptome by collecting cytoplasmic and nucleic RNA while performing step (i) and creating an RNA-Seq library using the collected RNA.

In an aspect. purifying and tagmenting DNA can comprise one or more of the following: isolating nuclei from a population of cells; incubating the isolated nuclei with an assembled Tn5 transposome; digesting the isolated nuclei with a first restriction enzyme; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with a second restriction enzyme; circularizing the digested DNA and purifying the circularized DNA; digesting the purified DNA with a third restriction enzyme, or any combination thereof. In an aspect, purifying and tagmenting DNA can comprise isolating nuclei from a population of cells; incubating the isolated nuclei with an assembled Tn5 transposome; digesting the isolated nuclei with a first restriction enzyme; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with a second restriction enzyme; circularizing the digested DNA and purifying the circularized DNA; digesting the purified DNA with a third restriction enzyme, or any combination thereof.

In an aspect of a disclosed method, analyzing chromatin structure and function can comprise one or more of the following: isolating nuclei from a population of cells; incubating the isolated nuclei with an assembled Tn5 transposome; digesting the isolated nuclei with a first restriction enzyme; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with a second restriction enzyme; circularizing the digested DNA and purifying the circularized DNA: digesting the purified DNA with a third restriction enzyme; performing PCR to generate DNA libraries, or any combination thereof, wherein the method identifies cis-regulatory chromatin interactions and characterizes chromatin accessibility. In an aspect, a disclosed method can comprise isolating nuclei from a population of cells; incubating the isolated nuclei with an assembled Tn5 transposome; digesting the isolated nuclei with a first restriction enzyme; incubating the digested nuclei with a splint oligonucleotide: ligating in situ the Tn5 adaptors to the proximal genomic DNA; reversing the crosslink: purifying the reverse cross-linked DNA and dissolving the purified DNA; digesting the purified DNA with a second restriction enzyme; circularizing the digested DNA and purifying the circularized DNA; digesting the purified DNA with a third restriction enzyme; and performing PCR to generate DNA libraries, wherein the method identifies cis-regulatory chromatin interactions and characterizes chromatin accessibility. In an aspect, the steps in a disclosed method can be performed in the order as listed.

In an aspect, analyzing the transcriptome can comprise one or more of the following: combining supernatant comprising cytoplasmic RNA and supernatant comprising nucleic RNA; reversing the crosslink; purifying the reverse crosslinked RNA: dissolving the purified RNA; treating the purified RNA with DNase; creating an RNA-Seq library, or any combination thereof. In an aspect, analyzing the transcriptome can comprise combining supernatant comprising cytoplasmic RNA and supernatant comprising nucleic RNA; reversing the crosslink; purifying the reverse crosslinked RNA; dissolving the purified RNA; treating the purified RNA with DNase; and creating an RNA-Seq library. RNA-Seq and RNA-Seq protocols are well-known to the art. In an aspect, creating an RNA-Seq library can comprise using a smartseq2 protocol. In an aspect. the steps of a disclosed method of analyzing the transcriptome can be performed in the order as listed.

In an aspect, a disclosed method of performing a multi-omics assay can further comprise processing the resulting datasets. In an aspect. processing the resulting datasets can comprise mapping and visualizing the uniquely mapped paired-end tags using a bioinformatics software program for visualizing molecular interactions, generating a comprehensive map of cis-regulatory chromatin contacts, calculating a cumulative interactive score for each resulting interaction anchor, or any combination thereof. In an aspect, a disclosed method can identify chromatin interactions that are enriched across multiple chromatin states. In an aspect, multiple chromatin states can comprise enhancers, promoters, and regions associated with active, poised, bivalent, and repressed chromatin states.

In an aspect, a disclosed restriction enzyme can comprise a restriction site of 1, 2, 3, 4, 5, 6, or 8 bases long. In an aspect of a disclosed method performing a multi-omics assay, the first, second, and third restriction enzymes are the same. In an aspect of a disclosed method, the first, second, and third restriction enzymes are different. In an aspect of a disclosed method, two of the first, second, and third restriction enzymes are the same. Restriction enzymes suitable for a disclosed method performing a multi-omics assay are disclosed infra. In an aspect, a disclosed restriction enzyme can comprise a 4 bp cutter. 4 bp cutters suitable for a disclosed method performing a multi-omics assay are disclosed infra. In an aspect, a 4 bp cutter can provide better data resolution than, for example, a 6 bp cutter or a 8 bp cutter. In an aspect, a first disclosed restriction enzyme can be CviQI. In an aspect, a second disclosed restriction enzyme can be NIaIII. In an aspect, a third disclosed restriction enzyme can be PmeI. In an aspect, a disclosed first restriction enzyme can be CviQI, the second restriction enzyme can be NIaIII, and the third restriction enzyme can be PmeI. In an aspect, a disclosed method can use any combination of 4 bp cutters.

In an aspect, a disclosed population of cells can be cross-linked. Crosslinking is known to the art and crosslinking cells to preserve protein-chromatin interactions is also known to the art. Further, crosslinking protocols are also known to the art and are discussed infra. In an aspect, a disclosed crosslinking protocol can comprise washing the population of cells with PBS, contacting the cells with accutase, removing the accutase, resuspending the cells with Dulbecco's Modified Eagle Medium (DMEM), contacting the cells with fixative agent, contacting the cells with glycine, pelleting the crosslinked cells by centrifugation, and washing the pelleted crosslinked cells using PBS. Fixative agents suitable for use in a disclosed method performing a multi-omics assay are disclosed infra. In an aspect, a disclosed fixative agent can comprise formaldehyde.

In an aspect, a disclosed isolating step can comprise incubating the cells in a buffer comprising bovine serum albumin (BSA), dithiothreitol (DTT), and IGEPAL.

In an aspect, a disclosed isolating can further comprise centrifuging the cells to isolate the nuclei and collecting the supernatant comprising cytoplasmic RNA. In an aspect, a disclosed incubating step can further comprise centrifuging the isolated nuclei and collecting the supernatant comprising the nucleic RNA.

In an aspect, a disclosed Tn5 transposome can be a pre-assembled Tn5 transposome. In an aspect, a disclosed method can comprise assembling the Tn5 transposome. In an aspect, assembling a disclosed Tn5 transposome can comprise annealing two Tn5 adaptors and incubating the annealed Tn5 adaptors with a Tn5 transposase. In an aspect, a disclosed Tn5 adaptor can comprise the sequence set forth in SEQ ID NO:01. In an aspect, a disclosed Tn5 adaptor can comprise the sequence set forth in SEQ ID NO:02. In an aspect, a disclosed Tn5 adaptor can comprise the sequence set forth in SEQ ID NO:01 and the other Tn5 adaptor can comprise the sequence set forth in SEQ ID NO:02. In an aspect a disclosed Tn5 adaptor can comprise a Mosaic End sequence for Tn5 recognition and a single-stranded flanking sequence that ligates to CviQI-digested DNA fragment using a splint oligonucleotide. In an aspect, a skilled person can craft a Tn5 adaptor. In an aspect, a Tn5 adaptor for use in a disclosed method can comprise a ME sequence and a reverse complement sequence to the splint oligonucleotide and can have the ability to ligate to the restriction enzyme digested genomic DNA.

In an aspect of a disclosed method of performing a multi-omics assay, a disclosed splint oligonucleotide can comprise the sequence set forth in SEQ ID NO:03. In an aspect, the ligating in situ step of a disclosed method can comprise using a T4 DNA ligase and a ligation buffer (such as, for example, a T4 ligation buffer). In an aspect, a skilled person can craft a splint oligonucleotide. In an aspect, a splint oligonucleotide for use in a disclosed method can comprise a reverse complement sequence to the Tn5 adaptor. In an aspect, a disclosed splint oligonucleotide/Tn5 adaptor can have the ability to ligate to the restriction enzyme digested genomic DNA.

In an aspect, the reversing the crosslink step of a disclosed method can comprise resuspending the nuclei in Tris-HCL, Proteinase K, and NaCl. In an aspect, the purifying the reverse cross-linked DNA step of a disclosed method can comprise a phenol:chloroform:isoamyl alcohol treatment followed by ethanol precipitation.

In an aspect, a disclosed method can further comprise repairing the Tn5 transposition gap. In an aspect, repairing the Tn5 transposition gap can comprise incubating the purified DNA with dNTPs and a DNA polymerase (such as, for example, a T4 DNA polymerase). DNA polymerases are known to the art and disclosed supra.

In an aspect of a disclosed method of performing a multi-omics assay, performing PCR step can comprise mixing the digested purified DNA with dNTPs, a forward primer, a reverse primer, and a polymerase. In an aspect, a disclosed forward primer can comprise the sequence set forth in SEQ ID NO:04 and wherein the reverse primer can comprise the sequence set forth in SEQ ID NO:05. In an aspect, a skilled person can craft one or more primers for use in a disclosed method. In an aspect, a primer for use in a disclosed kit can amplify DNA from Tn5 inserted regions. In an aspect, a primer for use in a disclosed kit can amplify DNA ligated to Tn5 adaptor.

In an aspect of a disclosed method of performing a multi-omics assay, the resulting amplified chimeric DNA fragment can contain one end derived from the CviQI digested genomic DNA and one end derived from the Tn5-tagmented open chromatin sequence. In an aspect of a disclosed method, the end derived from disclosed CviQI digested genomic DNA can be captured by Read 1 of each pair-end sequence. In an aspect of a disclosed method, the end derived from disclosed Tn5-tagmented open chromatin sequence can be captured by Read 2 of each pair-end sequence. In an aspect of a disclosed method, the end derived from disclosed CviQI digested genomic DNA can be captured by Read 1 of each pair-end sequence while the end derived from disclosed Tn5-tagmented open chromatin sequence can be captured by Read 2 of each pair-end sequence.

In an aspect, a disclosed method of performing a multi-omics assay can comprise using gel extraction to obtain those PCR products having a size of about 400-600 bp. In an aspect, the gel extracted PCR products can be subjected to deep sequencing. Gel extraction techniques are known to the art. In an aspect, gel extracted PCR products can be subjected to deep sequencing. As known to the art. deep sequencing is synonymous with next generation sequencing and refers to sequencing a genomic region multiple times (e.g., sometimes hundreds or even thousands of times). Deep sequencing protocols are known to the art.

In an aspect, a disclosed method does not comprise (or can exclude) antibody-mediated immunoprecipitation, adaptor ligation, biotin pulldown, or any combination thereof.

In an aspect, a disclosed population of cells can comprise at least 75,000 cells, at least 80,000 cells, at least 85,000 cells, at least 90,000 cells, at least 95,000 cells, at least 100,000 cells, at least 105,000 cells, at least 110,000 cells, at least 115,000 cells, at least 120,000 cells, or at least 125,000 cells. In an aspect, a disclosed population of cells can comprise about 75,000 to about 125,000 cells or can comprise about 100,000 cells.

In an aspect, a disclosed population of cells can comprise cells obtained from a biosample and then subjected to a crosslinking protocol. Crosslinking protocols are known to the art. In an aspect of a disclosed method, a disclosed crosslinking protocol can comprise washing the cells obtained from the biosample with PBS, contacting the cells with a digestion agent (such as, for example, accutase, collagenase, liberase, trypsin, TrypLE, non-enzymatic cell dissociation solution (NECDS)), removing the digestion agent, resuspending the cells with Dulbecco's Modified Eagle Medium (DMEM), contacting the cells with fixative agent, contacting the cells with glycine, pelleting the crosslinked cells by centrifugation, and washing the pelleted crosslinked cells using PBS.

In an aspect, a disclosed population of cells can be obtained from any number of sources or samples. For example, a disclosed biosample comprising cells for use in a disclosed method can be obtained from a subject by any number of means known to the art, including by obtaining or harvesting bodily fluids (e.g., blood, tears, urine, CSF. serum, lymph, mucus, saliva, anal and vaginal secretions, perspiration, and semen), taking tissue (e.g., a biopsy, graft, etc.), and/or by collecting cells. In an aspect, a disclosed population of cells can comprise a single type of cell or multiple types of cells. In an aspect, a disclosed population of cells can be heterogenous or homogenous. A disclosed population of cells can comprise a singular type of organism or multiple types of organisms. In an aspect, a disclosed biosample can be obtained from a subject. In an aspect, a disclosed method can comprise obtaining a disclosed biosample from a subject. In an aspect, a disclosed method can comprise obtaining a population of cells from the subject's biosample. In an aspect, a disclosed biosample can comprise a low input clinical sample. In an aspect, a disclosed population of cells can comprise a low input clinical sample.

In an aspect, a subject can be diagnosed with or can be suspected of having a disease or disorder. In an aspect, a disease or disorder can be a disease or disorder associated with chromatin deregulation and/or chromatin dysregulation. Diseases or disorder associated with chromatin deregulation and/or chromatin dysregulation are known to the art and include but are not limited to Alzheimer's disease, Amyotrophic lateral sclerosis (ALS). Angelman syndrome, ATR-X syndrome, Brachydactyly mental retardation syndrome, cerebro-oculo-facio-skeletal syndrome (COFS), Chromatin remodeling CHARGE syndrome, Cockayne syndrome, Coffin-Siris syndrome, Facioscapulohumera muscular dystrophy (FSHD), Fragile X syndrome, Huntington's disease, Immunodeficiency, centromeric region instability, and facial anomalies syndrome (ICF), Juberg-Marsidi syndrome, Kabuki syndrome, Kleefstra syndrome, MRD12, MRD14, MRD15, MRD16, Parkinson's disease, Prader-Willi syndrome, Rett syndrome, Rubinstein-Taybi syndrome, Smith-Fineman-Myers syndrome, Sotos syndrome, Sutherland-Haan syndrome, Weaver syndrome, and X-linked mental retardation.

In an aspect, a subject can be diagnosed with or can be suspected of having a disease or disorder affected by a gene having chromatin deregulation and/or chromatin dysregulation. Such diseases or disorders are known to the art and include but are not limited to 15q11-q13 locus. A2aR, APOE, ARID1A (BAF250A), ARID1B (BAF250B), ATRX (RAD54L), CHD7, CREBBP (CBP, KAT3A), DNMT3B, EHMT1 (GLP, KMT1D), EP300 (KAT3B), ERCC6 (CSB), EZH2 (KMT6), FMR1, FSHD locus 4q35, FUS (TLS), HDAC4, JARID1C (SMCX, KDM5C), MARCB1 (BAF47, SNF5L1), MECP2, MLL2 (KMT2B), NSD1 (KMT3B), PHF8, SCA7 locus, SMARCA2 (BRM, BAF190B, SNF2A), SMARCA4 (BRG1, BAF190A, SNF2B), SNCA (alpha-synuclein), TNFA (TNF-alpha), UBE3A (E6AP), and UTX (KDM6A).

In an aspect, a subject can be diagnosed with or can be suspected of having a critical limb ischemia (CLI).

In an aspect, a disclosed method of performing a multi-omics assay can comprise repeating the steps using a second population of cells. In an aspect, a disclosed second population of cells can comprise cells obtained from a disclosed second biosample and then can then be subjected to a crosslinking protocol. In an aspect, a disclosed second biosample can be obtained from a subject. In an aspect, a disclosed biosample can be obtained from a subject not having been diagnosed with or not suspected of having a disease or disorder.

In an aspect of a disclosed method of performing a multi-omics assay can further comprise processing the resulting datasets. In an aspect, a disclosed method can further comprise comparing the datasets obtained from the first population of cells to the datasets obtained from the second population of cells. In an aspect, a disclosed method can comprise measuring differences in the cis-regulatory chromatin interactions, the chromatin accessibility, the transcriptome, or any combination thereof between the two populations of cells.

In an aspect, processing the datasets for a disclosed second population of cells (or any populations of cells) can comprise mapping and visualizing the uniquely mapped paired-end tags using a bioinformatics software program for visualizing molecular interactions for a disclosed second population of cells. generating a comprehensive map of cis-regulatory chromatin contacts a disclosed second population of cells, or any combination thereof. For example, in an aspect, a disclosed method of performing a multi-omics assay can capture the number of active-to-active interactions to the number of inactive-to-inactive interactions in one or more populations of cells, or comparing the interaction strength/confidence of the active-to-active interactions to interaction strength/confidence of the inactive-to-inactive interactions in one or more populations of cells, or comparing the transcriptional/enhancer activity of the active-to-active interactions to the transcriptional/enhancer activity of the inactive-to-inactive interactions in one or more populations of cells, or any combination thereof.

In an aspect, a disclosed method of performing a multi-omics assay can generate about 10-fold to about 20-fold more cis-paired-end tags than Trac-looping or can generate about 15-fold to about 18-fold more cis-paired-end tags than Trac-looping.

In an aspect, a disclosed method can generate greater than 200 million pair-end raw reads, or about 250 million to about 350 million pair-end raw reads, or about 300 million pair-end raw reads, or greater than 300 million pair-end raw reads. In an aspect, a disclosed method can generate about 100 million to about 200 million uniquely mapped paired-end tags, or more than 100 million uniquely mapped paired-end tags, or more than 200 million uniquely mapped paired-end tags.

In an aspect of a disclosed method, the resolution of the cis-regulatory chromatin contacts can comprise about 5 KB, about 10 KB, about 15 KB, about 20 KB. or greater than 20 KB. In an aspect of a disclosed method, the resolution of the cis-regulatory chromatin contacts can comprise about 5 KB.

In an aspect, a disclosed Tn5 transposome can be a pre-assembled Tn5 transposome. In an aspect, a disclosed method can further comprise assembling a Tn5 transposome prior to a disclosed incubating step. In an aspect, assembling a disclosed Tn5 transposome can comprise annealing a first Tn5 adaptor and a second Tn5 adaptor and mixing the annealed Tn5 adaptor with Tn5 transposase. In an aspect, a disclosed method can further comprise purifying Tn5 transposase from transformed bacteria carrying a Tn5 expression plasmid.

In an aspect, a disclosed method can further comprise integrating public epigenome datasets into a disclosed processing step.

In an aspect, processing a disclosed resulting dataset can comprise using a distiller pipeline. In an aspect, a disclosed distiller pipeline can comprise one or more of the following: aligning the reads to hg38 reference genome using bwa mem with flags -SP; parsing the alignments; generating paired end tags (PET) using the pairtools; filtering out PETs with low mapping quality (MAPQ <10); removing PETs with the same coordinate on the genome or mapped to the same digestion fragment; flipping uniquely mapped PETs as side 1 with the lower genomic coordinate; aggregating the flipped uniquely mapped PETs into contact matrices in the cooler format using the cooler tools at delimited resolution; extracting dense matrix data from cooler files; visualizing the dense matrix data using HiGlass, or any combination thereof. In an aspect, a disclosed distiller pipeline can comprise aligning the reads to hg38 reference genome using bwa mem with flags -SP; parsing the alignments; generating paired end tags (PET) using the pairtools: filtering out PETs with low mapping quality (MAPQ <10); removing PETs with the same coordinate on the genome or mapped to the same digestion fragment: flipping uniquely mapped PETs as side 1 with the lower genomic coordinate; aggregating the flipped uniquely mapped PETs into contact matrices in the cooler format using the cooler tools at delimited resolution; extracting dense matrix data from cooler files; and visualizing the dense matrix data using HiGlass. In an aspect, a disclosed method can comprise calculating the R1 and R2 reads signal around TSS or peaks prior to PET flipping.

In an aspect of a disclosed method of performing a multi-omics assay, the similarity between different Hi-C datasets can be measured by HiCRep (described by Yang T, et al. (2017) Genome Res. 27:1939-1949). In an aspect, the stratum adjusted correlation coefficient (SCC) can be calculated on a per chromosome basis using HiCRep on 100 KB resolution data with a max distance of 5 Mb. In an aspect, the SCC can be calculated as a weighted average of stratum-specific Pearson's correlation coefficients.

In an aspect of a disclosed method of performing a multi-omics assay, compartmentalization, directionality index, and insulation score can be assessed using cooltools (see https://github.com/mirnylab/cooltools). Briefly, eigenvector decomposition can be performed on cis contact maps at 100 KB resolution. The first three eigenvectors and eigenvalues can be calculated, and the eigenvector associated with the largest absolute eigenvalue can be chosen. An identically binned track of GC content can be used to orient the eigenvectors. The insulation score and directionality Index can be computed by cooltools using ‘find_insulating_boundaries’ and ‘directionality’ function, respectively.

In an aspect of a disclosed method of performing a multi-omics assay, the curves of contact probability as a function of genomic separation can be generated by pairsqc following the 4DN pipeline (see https://github.com/4dn-dcic/pairsqc). Briefly, the genome can be binned at log 10 scale at interval of 0.1. For each bin, contact probability can be computed as number of reads/number of possible reads/bin size.

To process the RNA profile data, reads can be aligned to hg38 genome with Hisat2 (Kim D, et al. (2019) Nat. Biotechnol. 37:907-915) using hg38 genome_tran index obtained from Hisat2 website (http://daehwankimlab.github.io/hisat2/download/). Raw reads for each gene can be quantified using featureCounts.

To process 1D open chromatin peak in a disclosed method, unique mapped DNA library R2 reads can be extracted before PET flipping. R2 reads from long range (>20 KB) and the inter-chromosome trans-PETs can be combined and processed to be compatible as MACS2 input BED files. R2 reads from the short-range cis-PETs can be discarded to avoid the potential bias due to proximity to CviQI enzyme cut sites (Lareau CA, et al. (2018) Nature Methods. 15:155-156). MACS2 can be used to identify ATAC peaks following the ENCODE pipeline (see https://github.com/ENCODE-DCC/atac-seq-pipeline) with the following parameters: “-q 0.01 --shift 150 --extsize -75--nomodel -B --SPMR --keep-dup all”.

In an aspect of a disclosed method of performing a multi-omics assay, a CTCF ChIP-seq peak list of H1 can be downloaded from ENCODE (accession No. ENCFF821AQO) and searched for CTCF sequence motifs using gimme (Van Heeringen S J, et al. (2011) Bioinformatics. 27:270-271) and CTCF motif (MA0139.1) from the JASPAR database (FOrnes O, et al. (2020) Nucleic Acid Res. 48:D87-D92). In an aspect of a disclosed method, a subset of interactions with both ends containing either a single CTCF motif or multiple CTCF motifs in the same direction can be selected. In an aspect, the frequency of all possible directionality of CTCF motif pairs, convergent, tandem and divergent can be evaluated.

In an aspect, a disclosed method of performing a multi-omics assay can comprise chromatin interaction calling. In an aspect, HiCAR, PLAC-seq, and HiChIP datasets can be used. In an aspect, a disclosed method can use MAPS to call the significant chromatin interactions. In an aspect. paired-end tags can first be extracted from cooler datasets at 5 KB or 10 KB resolution using the “cooler dump” function with parameters: “-t pixels -H --join”. In an aspect, interaction anchor bins can be defined by the ATAC peaks or corresponding ChIP-seq peaks called using MACS2. MAPS can apply a positive Poisson regression-based approach to normalize systematic biases from restriction enzyme cut sites, GC content, sequence mappability, and 1D signal enrichment. In an aspect, interactions that are located within 15 KB of each other at both ends into clusters can be grouped and all other interactions can be classified as singletons. In an aspect, interactions with 6 or more and normalized contact frequency (raw read counts/expected read counts) >=2 can be retained and the significant interactions can be defined by FDR <0.01 for clusters and FDR <0.0001 for singletons. In an aspect of a disclosed method that addresses the situ Hi-C dataset, the hic file can be downloaded from 4DN data portal (accession No. 4DNES2MSJIGV) and HiCCUPS can be applied to call interactions at 10 KB resolution with the following parameters: “-r 10000 -k KR -f.1,.1 -p 4,2 -i 7,5 -t 0.02,1.5,1.75,2 -d 20000,20000”.

In an aspect of a disclosed method of performing a multi-omics assay, chromatin state calls can be obtained from the Roadmap Epigenomics Mapping Consortium. In an aspect, chromatin state calls can comprise an 18-state model. To determine which pairs of chromatin states were enriched at interaction anchors at a statistically significant level, the distribution of chromatin states can be examined at interaction anchors using HOMER. In an aspect. it can be assessed whether a connection between the feature is over-represented or under-represented given the general enrichment for each chromatin states at the interaction anchors. In an aspect, the HOMER “annotateInteractions” function can be used to obtain the p value and enrichment fold ratio for all pairs of chromatin states. The FDR adjusted p values can be obtained using the p.adjust function from the R package, with option method=“fdr”.

In an aspect, the enrichment for chromatin interactions in significant eQTL-TSS association can be tested. In an aspect, the eQTL-TSS associations can be obtained. To assess the significance of the enrichment, in an aspect, a null distribution can be generated by creating a simulated interaction datasets by resampling the same number of interactions at random from distance-matched interactions (with 10,000 repeats). In an aspect, the empirical P-value can be computed by comparing the observed overlapping number with the null distribution.

In an aspect of a disclosed method of performing a multi-omics assay, epigenetic features can be collected from a public database or consortium (e.g., the ENCODE consortium). In an aspect, average bigWig signals on each 5 KB anchor can be computed using the bigWigAverageOverBed command from UCSC. In an aspect, regression-based machine learning can be employed in a disclosed method. For regression, in an aspect, a sigmoid function can be used to scale the chromatin interaction score into a [0,1] range:

$$f ( x ) = \frac{1}{1 + {e}^{- c 1 ( x - c 2 )}}$$

In an aspect, c1 can be set to 0.05 and c2 can be set to 20 empirically, such that the bins with stronger interactions can have a value closer to 1 after sigmoid conversion. In an aspect, regression methods in the scikit-learn Python package can be used for regression analysis, including linear regression, decision tree, xbgboost, random forest and linear-kernel support vector machine (SVM). In an aspect, the XGBoost Python package can be used for XGBoost regression analysis.

In an aspect, a disclosed method of performing a multi-omics assay can comprise a gene ontology (GO) enrichment analysis. In an aspect, Clusterprofile can be used to examine whether particular gene sets are enriched in certain gene lists. In an aspect, GO categories with “BH” adjusted p value <0.05 can be considered significant.

Disclosed herein are methods of performing a multi-omics assay comprising identifying chromatin interactions and assessing chromatin accessibility, and sequencing RNA.

In an aspect, a disclosed identifying chromatin interactions and assessing chromatin accessibility step can comprise incubating isolated nuclei with an assembled Tn5 transposome; digesting the isolated nuclei with a restriction enzyme; incubating the digested nuclei with a splint oligonucleotide; ligating in situ the Tn5 adaptor to the proximal genomic DNA; reversing the crosslink; purifying the reverse cross-linked DNA and dissolving the purified DNA; digesting the purified DNA with a restriction enzyme; circularizing the digested DNA and purifying the circularized DNA; digesting the purified DNA with a restriction enzyme; performing PCR to generate DNA libraries.

In an aspect, a disclosed sequencing RNA step can comprise collecting supernatant comprising cytoplasmic RNA in a disclosed isolating step comprising centrifuging the cells to isolate the nuclei. In an aspect, a disclosed sequencing RNA step can further comprise collecting supernatant comprising the nucleic RNA in a disclosed incubating step of comprising centrifuging the isolated nuclei. In an aspect, a disclosed sequencing RNA step can comprise combining the supernatant comprising cytoplasmic RNA and the supernatant comprising nucleic RNA and reversing the crosslink. In an aspect, a disclosed sequencing RNA step can further comprise purifying the reverse crosslinked RNA, dissolving the purified RNA, and treating the purified RNA with DNase to remove DNA in solution. In an aspect, a disclosed sequencing RNA step can further comprise using a sample of the purified RNA to create an RNA-Seq library. RNA-Seq and RNA-Seq protocols are well-known to the art. In an aspect, creating an RNA-Seq library in a disclosed method can comprise using a smartseq2 protocol.

Disclosed herein are methods of performing a multi-omics assay comprising (i) identifying chromatin interactions and assessing chromatin accessibility, wherein identifying chromatin interactions and assessing chromatin accessibility comprises incubating isolated nuclei with an assembled Tn5 transposome; digesting the isolated nuclei with a restriction enzyme; incubating the digested nuclei with a splint oligonucleotide; ligating in situ the Tn5 adaptor to the proximal genomic DNA; reversing the crosslink; purifying the reverse cross-linked DNA and dissolving the purified DNA; digesting the purified DNA with a restriction enzyme; circularizing the digested DNA and purifying the circularized DNA: digesting the purified DNA with a restriction enzyme; performing PCR to generate DNA libraries; and (ii) sequencing RNA, wherein sequencing RNA comprises collecting supernatant comprising cytoplasmic RNA; collecting supernatant comprising the nucleic RNA: combining the supernatant comprising cytoplasmic RNA and the supernatant comprising nucleic RNA and reversing the crosslink; purifying the reverse crosslinked RNA, dissolving the purified RNA, and treating the purified RNA with DNase to remove DNA in solution; and using the purified RNA to create an RNA-Seq library.

In an aspect, the identifying chromatin interactions and assessing chromatin accessibility step and the sequencing RNA step can be performed concurrently. In an aspect, the steps of a disclosed method are performed in the order as listed.

In an aspect, a disclosed method does not comprise antibody-mediated immunoprecipitation, adaptor ligation, biotin pulldown, or any combination thereof.

In an aspect, a disclosed restriction enzyme can comprise a restriction site of 1, 2, 3, 4, 5, 6, or 8 bases long. In an aspect of a disclosed method performing a multi-omics assay, the first, second, and third restriction enzymes are the same. In an aspect of a disclosed method, the first, second, and third restriction enzymes are different. In an aspect of a disclosed method, two of the first, second, and third restriction enzymes are the same. Restriction enzymes suitable for a disclosed method performing a multi-omics assay are disclosed supra. In an aspect, a disclosed restriction enzyme can comprise a 4 bp cutter. 4 bp cutters suitable for a disclosed method performing a multi-omics assay are disclosed infra. In an aspect, a first disclosed restriction enzyme can be CviQI. In an aspect, a second disclosed restriction enzyme can be NIaIII. In an aspect, a third disclosed restriction enzyme can be PmeI. In an aspect, a first disclosed restriction enzyme can be CviQI, a second disclosed restriction enzyme can be NIaIII, and a third disclosed restriction enzyme can be PmeI.

In an aspect, a disclosed population of cells can be crosslinked prior to incubating step of a disclosed method. Crosslinking is known to the art and crosslinking cells to preserve protein-chromatin interactions is also known to the art. Further, crosslinking protocols are also known to the art and are discussed supra. In an aspect, a disclosed crosslinking protocol can comprise washing the population of cells with PBS, contacting the cells with accutase, removing the accutase, resuspending the cells with Dulbecco's Modified Eagle Medium (DMEM), contacting the cells with fixative agent, contacting the cells with glycine, pelleting the crosslinked cells by centrifugation, and washing the pelleted crosslinked cells using PBS. Fixative agents suitable for use in a disclosed method performing a multi-omics assay are disclosed supra. In an aspect, a disclosed fixative agent can comprise formaldehyde.

In an aspect, the isolating step of a disclosed method can comprise incubating the cells in a buffer comprising bovine serum albumin (BSA), dithiothreitol (DTT), and IGEPAL. In an aspect, the isolating step of a disclosed method can further comprise centrifuging the cells to isolate the nuclei and collecting the supernatant comprising cytoplasmic RNA.

In an aspect, the incubating step of a disclosed method can further comprise centrifuging the isolated nuclei to stop the reaction and collecting the supernatant comprising the nucleic RNA.

In an aspect, a disclosed Tn5 transposome can be a pre-assembled Tn5 transposome. In an aspect, a disclosed method can further comprise assembling the Tn5 transposome. In an aspect, assembling the Tn5 transposome can comprise annealing two Tn5 adaptors and incubating the annealed Tn5 adaptors with a Tn5 transposase. In an aspect, a disclosed Tn5 adaptor can comprise the sequence set forth in SEQ ID NO:01. In an aspect, a disclosed Tn5 adaptor can comprise the sequence set forth in SEQ ID NO:02. In an aspect, disclosed Tn5 adaptors used in a disclosed can comprise the sequence set forth in SEQ ID NO:01 and SEQ ID NO:02. In an aspect a disclosed Tn5 adaptor can comprise a Mosaic End sequence for Tn5 recognition and a single-stranded flanking sequence that ligates to CviQI-digested DNA fragment using a splint oligonucleotide. In an aspect, a skilled person can craft a Tn5 adaptor. In an aspect, a Tn5 adaptor for use in a disclosed method can comprise a ME sequence and a reverse complement sequence to the splint oligonucleotide and can have the ability to ligate to the restriction enzyme digested genomic DNA.

In an aspect, a disclosed splint oligonucleotide can comprise the sequence set forth in SEQ ID NO:03. In an aspect, the ligating in situ step of a disclosed method can comprise using a T4 DNA ligase and a ligation buffer (such as, for example, a T4 ligation buffer). In an aspect, a skilled person can craft a splint oligonucleotide. In an aspect, a splint oligonucleotide for use in a disclosed method can comprise a reverse complement sequence to the Tn5 adaptor. In an aspect, a disclosed splint oligonucleotide/Tn5 adaptor can have the ability to ligate to the restriction enzyme digested genomic DNA.

In an aspect, the reversing the crosslink step of a disclosed method can comprise resuspending the nuclei in Tris-HCL, Proteinase K, and NaCl. In an aspect, the purifying the reverse cross-linked DNA step of a disclosed method can comprise a phenol:chloroform:isoamyl alcohol treatment followed by ethanol precipitation.

In an aspect, a disclosed method can further comprise repairing the Tn5 transposition gap. In an aspect, repairing the Tn5 transposition gap can comprise incubating the purified DNA with dNTPs and a DNA polymerase (such as, for example, a T4 DNA polymerase). DNA polymerases are known to the art and disclosed infra.

In an aspect of a disclosed method, performing PCR can comprise mixing the digested purified DNA with dNTPs, a forward primer, a reverse primer, and a polymerase. In an aspect, a disclosed forward primer can have the sequence set forth in SEQ ID NO:04. In an aspect, a disclosed reverse primer can comprise the sequence set forth in SEQ ID NO:05. In an aspect, a skilled person can craft one or more primers for use in a disclosed method. In an aspect, a primer for use in a disclosed kit can amplify DNA from Tn5 inserted regions. In an aspect, a primer for use in a disclosed kit can amplify DNA ligated to Tn5 adaptor.

In an aspect of a disclosed method, the resulting amplified chimeric DNA fragment can contain one end derived from the CviQI digested genomic DNA and one end derived from the Tn5-tagmented open chromatin sequence. In an aspect of a disclosed method, the end derived from disclosed CviQI digested genomic DNA can be captured by Read 1 of each pair-end sequence. In an aspect of a disclosed method, the end derived from disclosed Tn5-tagmented open chromatin sequence can be captured by Read 2 of each pair-end sequence. In an aspect of a disclosed method, the end derived from disclosed CviQI digested genomic DNA can be captured by Read 1 of each pair-end sequence while the end derived from disclosed Tn5-tagmented open chromatin sequence can be captured by Read 2 of each pair-end sequence.

In an aspect, a disclosed method can further comprise using gel extraction to obtain those PCR products having a size of about 400-600 bp. Gel extraction techniques are known to the art. In an aspect, gel extracted PCR products can be subjected to deep sequencing. As known to the art, deep sequencing is synonymous with next generation sequencing and refers to sequencing a genomic region multiple times (e.g., sometimes hundreds or even thousands of times). Deep sequencing protocols are known to the art.

In an aspect, the sequencing RNA step of a disclosed method of performing a multi-omics assay can comprise combining the supernatant comprising cytoplasmic RNA and the supernatant comprising nucleic RNA and reversing the crosslink. In an aspect, a disclosed method can further comprises purifying the reverse crosslinked RNA. In an aspect, a disclosed method can further comprise dissolving the purified RNA and treating the purified RNA with DNase to remove DNA in solution. In an aspect, a disclosed method can further comprise using a sample of the purified RNA to create an RNA-Seq library. RNA-Seq and RNA-Seq protocols are well-known to the art. In an aspect, creating an RNA-Seq library in a disclosed method can comprise using a smartseq2 protocol.

In an aspect, a disclosed population of cells can comprise at least 75,000 cells, at least 80,000 cells, at least 85,000 cells, at least 90,000 cells, at least 95,000 cells, at least 100,000 cells, at least 105,000 cells, at least 110,000 cells, at least 115,000 cells, at least 120,000 cells, or at least 125,000 cells. In an aspect, a disclosed population of cells can comprise about 75,000 to about 125,000 cells or can comprise about 100,000 cells.

In an aspect, a disclosed population of cells can comprise cells obtained from a biosample and then subjected to a crosslinking protocol. Crosslinking protocols are known to the art. In an aspect of a disclosed method, a disclosed crosslinking protocol can comprise washing the cells obtained from the biosample with PBS, contacting the cells with a digestion agent (such as, for example, accutase, collagenase, liberase, trypsin. TrypLE, non-enzymatic cell dissociation solution (NECDS)), removing the digestion agent, resuspending the cells with Dulbecco's Modified Eagle Medium (DMEM), contacting the cells with fixative agent, contacting the cells with glycine, pelleting the crosslinked cells by centrifugation, and washing the pelleted crosslinked cells using PBS.

In an aspect, a disclosed population of cells can be obtained from any number of sources or samples. For example, a disclosed biosample comprising cells for use in a disclosed method can be obtained from a subject by any number of means known to the art, including by obtaining or harvesting bodily fluids (e.g., blood, tears, urine, CSF, serum, lymph, mucus, saliva, anal and vaginal secretions, perspiration, and semen), taking tissue (e.g., a biopsy, graft, etc.), and/or by collecting cells. In an aspect, a disclosed population of cells can comprise a single type of cell or multiple types of cells. In an aspect, a disclosed population of cells can be heterogenous or homogenous. A disclosed population of cells can comprise a singular type of organism or multiple types of organisms. In an aspect, a disclosed second biosample can be obtained from a subject. In an aspect, a disclosed method can comprise obtaining a disclosed biosample from a subject. In an aspect, a disclosed method can comprise obtaining a population of cells from the subject's biosample. In an aspect, a disclosed biosample can comprise a low input clinical sample. In an aspect, a disclosed population of cells can comprise a low input clinical sample.

In an aspect, a subject can have been diagnosed with or can be suspected of having a disease or disorder. In an aspect, a disease or disorder can be a disease or disorder associated with chromatin deregulation and/or chromatin dysregulation. Diseases or disorder associated with chromatin deregulation and/or chromatin dysregulation are known to the art and are discussed supra. In an aspect, a subject can be diagnosed with or can be suspected of having a disease or disorder having a gene affected by chromatin deregulation and/or chromatin dysregulation. Such diseases or disorders are known to the art and are discussed supra. In an aspect, a subject can be diagnosed with or can be suspected of having a critical limb ischemia (CLI).

In an aspect, a disclosed method can comprise subjecting a disclosed population of cells to a crosslinking protocol.

In an aspect, a disclosed method can further comprise repeating one or more steps of the method using a second population of cells. In an aspect, a disclosed method can further comprise repeating all the steps of the method using a disclosed population of cells. In an aspect, a disclosed second population of cells can comprise cells obtained from a disclosed second biosample and then subjected to a crosslinking protocol. In an aspect, a disclosed second population of cells can be obtained from any number of sources or samples. For example, a disclosed second biosample comprising cells for use in a disclosed method can be obtained from a subject by any number of means known to the art, including by obtaining or harvesting bodily fluids (e.g., blood, tears, urine, CSF, serum, lymph, mucus, saliva, anal and vaginal secretions, perspiration, and semen), taking tissue (e.g., a biopsy, graft, etc.), and/or by collecting cells. In an aspect, a disclosed second population of cells can comprise a single type of cell or multiple types of cells. In an aspect, a disclosed second population of cells can be heterogenous or homogenous. A disclosed second population of cells can comprise a singular type of organism or multiple types of organisms. In an aspect, a disclosed method can comprise obtaining a disclosed second biosample from a subject. In an aspect, a disclosed method can comprise obtaining a disclosed second population of cells from the subject's biosample. In an aspect, a disclosed biosample can comprise a low input clinical sample. In an aspect, a disclosed population of cells can comprise a low input clinical sample.

In an aspect, a disclosed second biosample can be obtained from a subject. In an aspect, a disclosed second biosample can be obtained from a subject not having been diagnosed with or not suspected of having a disease or disorder. In an aspect, a disclosed second biosample can be obtained from a subject having been diagnosed with or is suspected of having a disease or disorder. In an aspect, a disclosed second biosample can be obtained from the same subject that provided the disclosed first biosample. In an aspect, the first and second disclosed populations of cells can be obtained from the same subject. In an aspect, the first and second disclosed populations of cells can be obtained from different subjects. In an aspect, the first and second disclosed populations of cells can be obtained from the same subject, wherein the disclosed first population can be obtained prior to a treatment and wherein the disclosed second population can be obtained after the treatment.

In an aspect, a disclosed method of performing a multi-omics assay can comprise repeating one or more steps of the method using additional populations of cells (e.g., a third population, a fourth population, a fifth population, etc.). In an aspect, a disclosed method can be repeated one or more times using a new population of cells each time the method is repeated. In an aspect, a disclosed method can be used to compare chromatin interactions and chromatin accessibility across multiple populations of cells (e.g., a first population, a second population, a third population, a fourth population, so forth and so on). In an aspect, a disclosed method can be used to compare RNA-Seq data across multiple populations of cells (e.g., a first population, a second population, a third population, a fourth population, so forth and so on). In an aspect, a disclosed method can be used to compare RNA-Seq data to a pre-existing database.

In an aspect, a disclosed population of cells can comprise cultured cells. In an aspect, a first disclosed population of cells can comprise cultured cells, a second disclosed population of cells can comprise cultured cells, or both a first disclosed population and a second disclosed population of cells can comprise cultured cells. In an aspect, a disclosed population of cultured cells can comprise wild-type, normal, non-diseased, and/or non-disordered cells. In an aspect, a disclosed population of cultured cells can comprise mutant, atypical, diseased, and/or disordered cells. In an aspect, disclosed cultured cells can be mESCs, GM12878 cells, and/or H1 hESCs.

In an aspect, a disclosed method of performing a multi-omics assay can further comprise processing the resulting datasets concerning chromatin interactions and chromatin accessibility. In an aspect, processing the datasets can comprise mapping and visualizing the uniquely mapped paired-end tags using a bioinformatics software program for visualizing molecular interactions, generating a comprehensive map of cis-regulatory chromatin contacts, calculating a cumulative interactive score for each interaction anchor, or any combination thereof. In an aspect, a disclosed method can comprise comparing the resulting chromatin datasets obtained from the first population of cells to the datasets obtained from the second population of cells. In an aspect, a disclosed method can comprise comparing the resulting chromatin datasets obtained from multiple population of cells. In an aspect, a disclosed method can comprise comparing a resulting chromatin dataset obtained from a first population to chromatin dataset obtained from multiple population of cells (e.g., a second population, a third population, a fourth population, a fifth population, etc.).

In an aspect, a disclosed method can further comprise identifying transcriptome differences between the two or more, three or more, four or more, five or more, or more than five populations of cells.

In an aspect, a disclosed method of performing a multi-omics assay can further comprise identifying differences in cis-regulatory chromatin interactions and in chromatin accessibility between two or more, three or more, four or more, five or more, or more than five populations of cells.

In an aspect, a disclosed method can generate about 10-fold to about 20-fold more cis-paired-end tags than Trac-looping or can generate about 15-fold to about 18-fold more cis-paired-end tags than Trac-looping.

In an aspect, a disclosed method can generate greater than 200 million pair-end raw reads, or about 250 million to about 350 million pair-end raw reads, or about 300 million pair-end raw reads, or greater than 300 million pair-end raw reads. In an aspect, a disclosed method can generate about 100 million to about 200 million uniquely mapped paired-end tags, or more than 100 million uniquely mapped paired-end tags, or more than 200 million uniquely mapped paired-end tags.

In an aspect of a disclosed method, the resolution of the cis-regulatory chromatin contacts can comprise about 5 KB, about 10 KB, about 15 KB, about 20 KB. or greater than 20 KB. In an aspect of a disclosed method, the resolution of the cis-regulatory chromatin contacts can comprise about 5 KB.

In an aspect, a disclosed method of performing a multi-omics assay can capture “active-to-active” interactions and/or “inactive-to-inactive” interactions in one or more populations of cells. For example, in an aspect, a disclosed method can capture the number of active-to-active interactions to the number of inactive-to-inactive interactions in one or more populations of cells, or comparing the interaction strength/confidence of the active-to-active interactions to interaction strength/confidence of the inactive-to-inactive interactions in one or more populations of cells, or comparing the transcriptional/enhancer activity of the active-to-active interactions to the transcriptional/enhancer activity of the inactive-to-inactive interactions in one or more populations of cells, or any combination thereof.

In an aspect, a disclosed Tn5 transposome can be a pre-assembled Tn5 transposome. In an aspect, a disclosed method can further comprise assembling a Tn5 transposome prior to a disclosed incubating step. In an aspect, assembling a disclosed Tn5 transposome can comprise annealing a first Tn5 adaptor and a second Tn5 adaptor and mixing the annealed Tn5 adaptor with Tn5 transposase. In an aspect, a disclosed method can further comprise purifying Tn5 transposase from transformed bacteria carrying a Tn5 expression plasmid.

In an aspect, a disclosed method can further comprise integrating public epigenome datasets into a disclosed processing step.

In an aspect of a disclosed method, processing chromatin datasets can comprise using a distiller pipeline. Distiller pipelines are known to the art. For example, in an aspect, a disclosed method can comprise using a distiller pipeline found at https://github.com/mirnylab/distiller-nf. In an aspect, processing HiCAR datasets can comprise one or more of the following: aligning the reads to hg38 reference genome using bwa mem with flags -SP; parsing the alignments; generating paired end tags (PET) using the pairtools (e.g., https://github.com/mimylab/pairtools); filtering out PETs with low mapping quality (MAPQ <10); removing PETs with the same coordinate on the genome or mapped to the same digestion fragment; flipping uniquely mapped PETs as side 1 with the lower genomic coordinate; aggregating the flipped uniquely mapped PETs into contact matrices in the cooler format using the cooler tools at delimited resolution; extracting dense matrix data from cooler files; and visualizing the dense matrix data using HiGlass. In an aspect, a disclosed method can further comprise calculating the R1 and R2 reads signal around TSS or peaks prior to PET flipping.

In an aspect of a disclosed method of performing a multi-omics assay, the similarity between different Hi-C datasets can be measured by HiCRep (described by Yang T, et al. (2017) Genome Res. 27:1939-1949). In an aspect, the stratum adjusted correlation coefficient (SCC) can be calculated on a per chromosome basis using HiCRep on 100 KB resolution data with a max distance of 5 Mb. In an aspect, the SCC can be calculated as a weighted average of stratum-specific Pearson's correlation coefficients.

In an aspect of a disclosed method of performing a multi-omics assay. compartmentalization, directionality index, and insulation score can be assessed using cooltools (see https://github.com/mirnylab/cooltools). Briefly, eigenvector decomposition can be performed on cis contact maps at 100 KB resolution. The first three eigenvectors and eigenvalues can be calculated, and the eigenvector associated with the largest absolute eigenvalue can be chosen. An identically binned track of GC content can be used to orient the eigenvectors. The insulation score and directionality Index can be computed by cooltools using ‘find_insulating_boundaries’ and ‘directionality’ function, respectively.

In an aspect of a disclosed method of performing a multi-omics assay, the curves of contact probability as a function of genomic separation can be generated by pairsqc following the 4DN pipeline (see https://github.com/4dn-dcic/pairsqc). Briefly, the genome can be binned at log 10 scale at interval of 0.1. For each bin, contact probability can be computed as number of reads/number of possible reads/bin size.

To process the RNA profile data, reads can be aligned to hg38 genome with Hisat2 (Kim D, et al. (2019) Nat. Biotechnol. 37:907-915) using hg38 genome_tran index obtained from Hisat2 website (http://daehwankimlab.githab.io./hisat2/download/). Raw reads for each gene can be quantified using featureCounts.

To process 1D open chromatin peak in a disclosed method, unique mapped DNA library R2 reads can be extracted before PET flipping. R2 reads from long range (>20 KB) and the inter-chromosome trans-PETs can be combined and processed to be compatible as MACS2 input BED files. R2 reads from the short-range cis-PETs can be discarded to avoid the potential bias due to proximity to CviQI enzyme cut sites (Lareau C A. et al. (2018) Nature Methods. 15:155-156). MACS2 can be used to identify ATAC peaks following the ENCODE pipeline (see https://github.com/ENCODE-DCC-atac-seq-pipeline) with the following parameters: “-q 0.01 --shift 150 --extsize -75--nomodel -B --SPMR --keep-dup all”.

In an aspect of a disclosed method of performing a multi-omics assay, a CTCF ChIP-seq peak list of H1 can be downloaded from ENCODE (accession No. ENCFF82IAQO) and searched for CTCF sequence motifs using gimme (Van Heeringen S J, et al. (2011) Bioinformatics. 27:270-271) and CTCF motif (MA0139.1) from the JASPAR database (Fornes O, et al. (2020) Nucleic Acid Res. 48:D87-D92). In an aspect of a disclosed method, a subset of interactions with both ends containing either a single CTCF motif or multiple CTCF motifs in the same direction can be selected. In an aspect, the frequency of all possible directionality of CTCF motif pairs, convergent, tandem and divergent can be evaluated.

In an aspect, a disclosed method of performing a multi-omics assay can comprise chromatin interaction calling. In an aspect, HiCAR, PLAC-seq, and HiChIP datasets can be used. In an aspect, a disclosed method can use MAPS to call the significant chromatin interactions. In an aspect, paired-end tags can first be extracted from cooler datasets at 5 KB or 10 KB resolution using the “cooler dump” function with parameters: “-t pixels -H --join”. In an aspect, interaction anchor bins can be defined by the ATAC peaks or corresponding ChIP-seq peaks called using MACS2. MAPS can apply a positive Poisson regression-based approach to normalize systematic biases from restriction enzyme cut sites, GC content, sequence mappability, and 1D signal enrichment. In an aspect, interactions that are located within 15 KB of each other at both ends into clusters can be grouped and all other interactions can be classified as singletons. In an aspect, interactions with 6 or more and normalized contact frequency (raw read counts/expected read counts) >=2 can be retained and the significant interactions can be defined by FDR <0.01 for clusters and FDR <0.0001 for singletons. In an aspect of a disclosed method that addresses the situ Hi-C dataset, the .hic file can be downloaded from 4DN data portal (accession No. 4DNES2M5JIGV) and HiCCUPS can be applied to call interactions at 10 KB resolution with the following parameters: “-r 10000 -k KR -f 0.1,.1 -p 4,2 -i 7,5 -t 0.02,1.5,1.75,2 -d 20000,20000”.

In an aspect of a disclosed method of performing a multi-omics assay, chromatin state calls can be obtained from the Roadmap Epigenomics Mapping Consortium. In an aspect, chromatin state calls can comprise an 18-state model. To determine which pairs of chromatin states were enriched at interaction anchors at a statistically significant level, the distribution of chromatin states can be examined at interaction anchors using HOMER. In an aspect, it can be assessed whether a connection between the feature is over-represented or under-represented given the general enrichment for each chromatin states at the interaction anchors. In an aspect, the HOMER “annotateInteractions” function can be used to obtain the p value and enrichment fold ratio for all pairs of chromatin states. The FDR adjusted p values can be obtained using the p.adjust function from the R package. with option method=“fdr”.

In an aspect, the enrichment for chromatin interactions in significant eQTL-TSS association can be tested. In an aspect, the eQTL-TSS associations can be obtained. To assess the significance of the enrichment, in an aspect, a null distribution can be generated by creating a simulated interaction datasets by resampling the same number of interactions at random from distance-matched interactions (with 10,000 repeats). In an aspect, the empirical P-value can be computed by comparing the observed overlapping number with the null distribution.

In an aspect of a disclosed method of performing a multi-omics assay. epigenetic features can be collected from a public database or consortium (e.g., the ENCODE consortium). In an aspect, average bigWig signals on each 5 KB anchor can be computed using the bigWigAverageOverBed command from UCSC. In an aspect, regression-based machine learning can be employed in a disclosed method. For regression, in an aspect, a sigmoid function can be used to scale the chromatin interaction score into a [0,1] range:

$$f ( x ) = \frac{1}{1 + {e}^{- c 1 ( x - c 2 )}}$$

In an aspect, c1 can be set to 0.05 and c2 can be set to 20 empirically, such that the bins with stronger interactions can have a value closer to 1 after sigmoid conversion. In an aspect, regression methods in the scikit-learn Python package can be used for regression analysis, including linear regression, decision tree, xbgboost, random forest and linear-kernel support vector machine (SVM). In an aspect, the XGBoost Python package can be used for XGBoost regression analysis.

In an aspect, a disclosed method of performing a multi-omics assay can comprise a gene ontology (GO) enrichment analysis. In an aspect, Clusterprofile can be used to examine whether particular gene sets are enriched in certain gene lists. In an aspect, GO categories with “BH” adjusted p value <0.05 can be considered significant.

In an aspect, identifying chromatin interactions and assessing chromatin accessibility can comprise isolating nuclei from a population of cells; incubating isolated nuclei with an assembled Tn5 transposome; digesting the isolated nuclei with a first restriction enzyme; incubating the digested nuclei with a splint oligonucleotide; ligating in situ the Tn5 adaptor to the proximal genomic DNA; reversing the crosslink; purifying the reverse cross-linked DNA and dissolving the purified DNA; digesting the purified DNA with a second restriction enzyme; circularizing the digested DNA and purifying the circularized DNA: digesting the purified DNA with a third restriction enzyme; and performing PCR to generate DNA libraries.

In an aspect, identifying chromatin interactions and assessing chromatin accessibility can comprise isolating nuclei from a population of cells; incubating isolated nuclei with an assembled Tn5 transposome; digesting the isolated nuclei with CviQI; incubating the digested nuclei with a splint oligonucleotide; ligating in situ the Tn5 adaptor to the proximal genomic DNA; reversing the crosslink; purifying the reverse cross-linked DNA and dissolving the purified DNA; digesting the purified DNA with NIaIII; circularizing the digested DNA and purifying the circularized DNA; digesting the purified DNA with PmeI; and performing PCR to generate DNA libraries.

### C. Methods of Performing HiCAR

Disclosed herein is a method of performing a high-throughput chromosome conformation capture on accessible DNA and mRNA-Seq co-assay (HiCAR), the method comprising incubating isolated nuclei with an assembled Tn5 transposome; digesting the isolated nuclei with a first restriction enzyme; incubating the digested nuclei with a splint oligonucleotide; ligating in situ the Tn5 adaptor to the proximal genomic DNA; reversing the crosslink; purifying the reverse cross-linked DNA and dissolving the purified DNA; digesting the purified DNA with a second restriction enzyme; circularizing the digested DNA and purifying the circularized DNA; digesting the purified DNA with a third restriction enzyme; performing PCR to generate DNA libraries; and creating a RNA-Seq library, wherein the method identifies cis-regulatory chromatin interactions, characterizes chromatin accessibility, and analyzes the transcriptome in the population of cells.

Disclosed is a method of performing a high-throughput chromosome conformation capture on accessible DNA and mRNA-Seq co-assay (HiCAR), the method comprising isolating nuclei from a population of cells; incubating the isolated nuclei with an assembled Tn5 transposome; digesting the isolated nuclei with a first restriction enzyme; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with a second restriction enzyme; circularizing the digested DNA and purifying the circularized DNA: digesting the purified DNA with a third restriction enzyme; performing PCR to generate DNA libraries; and creating a RNA-Seq library, wherein the method identifies cis-regulatory chromatin interactions, characterizes chromatin accessibility, and analyzes the transcriptome in the population of cells.

Disclosed is a method of performing a high-throughput chromosome conformation capture on accessible DNA and mRNA-Seq co-assay (HiCAR), the method comprising isolating nuclei from a population of cells; incubating the isolated nuclei with an assembled Tn5 transposome; digesting the isolated nuclei with CviQI; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with NIaIII; circularizing the digested DNA and purifying the circularized DNA; digesting the purified DNA with PmeI; performing PCR to generate DNA libraries; and creating an RNA-Seq library, wherein the method identifies cis-regulatory chromatin interactions. characterizes chromatin accessibility, and analyzes the transcriptome in the population of cells.

Disclosed is a method of performing a high-throughput chromosome conformation capture on accessible DNA and mRNA-Seq co-assay (HiCAR), the method comprising incubating the isolated nuclei with an assembled Tn5 transposome: digesting the isolated nuclei with CviQI; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with NIaIII; circularizing the digested DNA and purifying the circularized DNA: digesting the purified DNA with PmeI; performing PCR to generate DNA libraries; and creating an RNA-Seq library wherein the method identifies cis-regulatory chromatin interactions, characterizes chromatin accessibility, and analyzes the transcriptome in the population of cells.

In an aspect, the steps of a disclosed method can be performed in the order as listed.

In an aspect, a disclosed method can further comprise processing the resulting HiCAR datasets. In an aspect, processing the HiCAR datasets can comprise mapping and visualizing the uniquely mapped paired-end tags using a bioinformatics software program for visualizing molecular interactions, generating a comprehensive map of cis-regulatory chromatin contacts, calculating a cumulative interactive score for each HiCAR interaction anchor, or any combination thereof. In an aspect. chromatin interactions identified by a disclosed method can be enriched across multiple chromatin states. In an aspect, the multiple chromatin states can comprise enhancers, promoters, and regions associated with active, poised, bivalent, and repressed chromatin states.

In an aspect, a disclosed method does not comprise antibody-mediated immunoprecipitation, adaptor ligation. biotin pulldown, or any combination thereof.

In an aspect, a disclosed restriction enzyme can comprise a restriction site of 1, 2, 3, 4, 5, 6, or 8 bases long. In an aspect of a disclosed method, the first, second, and third restriction enzymes are the same. In an aspect of a disclosed method, the first, second, and third restriction enzymes are different. In an aspect of a disclosed method, two of the first, second, and third restriction enzymes are the same.

In an aspect, a disclosed restriction enzyme can comprise AatII, Acc65I, AccI, AciI, AcII, AcuI, AfeI, AflIII, AflIII, AfIIII, AgeI, AhdI, AleI, AluI, AwI, AlwNI, ApaI, ApalI, ApeKI, ApoI, AscI, AseI, AsiSI, AvaI, AvalI, AvrII, BaeGI, BaeI, BamHI, BanI, BanII, BbsI, BbvCI, BbvI, BccI, BceAI, BcgI, BciVI, BclI, BfaI, BfuAI, BfuCI, BglH, BglII, BlpI, BmgBI, BmrI, BmtI, BpmI, Bpu10L, BpuE1, BsaA1, BsaBI, BsaHI, BsaI, BsaJI, BsaWI, BsaXI, BscRI, BscYI, BsgI, BsiEI, BsiHKAI, BsiWI, BslI, BsmAI, BsmBI, BsmFI, BsmI, BsoBI, Bspl286I, BspCNI, BspDI, BspEI, BspHLI, BspMI, BspQI, BsrBI, BsrD, BsrFL, BsrG, BsrI, BssHII, BssKL, BssS1, BstAPI, BstBI, BstEII, BstNI, BstUI, BstXI, BstYI, BstZ17I, Bsu36I, BtgI, BtgZI, BtsCI, BtsI, Cac8I, ClaI, CspCI, CviAII, CviKi-1, CviQI, DdcI, DpnI, DpnII, DraI, DraIII, DrdI, EacI, EagI, EarI, EciI, Eco53kI, EcoNI, EcoO109T, EcoP15I, EcoRI, EcoRV, FatI, FauI, Fnu4HI, FokI, FseI, FspI, HaelI, HaeIII, HgaI, HhaI, HincII, HindIII, HinfI, HinP1I, HpaI, HpaII, HphI, Hpy166II, Hpy188L, Hpy188III, Hpy991, HpyAV, HpyCH4III, HpyCH4IV, HpyCH4V, KasI, KpnI, MboI, MbolI, MfeI, MluI, MiyI, MmeI, MnII, MscI, MseI, MsII, MspAlI, MspI, MwoI, NaeI, NarI, Nb. BbvC1, Nb.Bsml, Nb.BsrDI, Nb.BtsT, NciI, NcoI, NdeI, NgoMIV, NheI, NIaIII, NlaTV, NmeAIII, NoI, NruI, NsiI, NspI, Nt.AlwI, Nt.BbvCL, Nt.BsmAL, Nt.BspQL Nt.BstNBI, Nt.CviPII, Pacl PaeR71, PciI, PflFIL PflMI, PhoI, PleI, PmeI, PmlI PpuML, PshAI, PsiI, PspGI, PspOMI, PspXI, PstT, PvuI, PvulI, RsaI, RsrlI, Sacl SaciI, SalI, SapI, Sau3AI, Sau96I, SbfI, ScaI, ScrFI, SexAI, SfaNL Sfc, SfiI, SfoL SgrAL SmaI, SmiI, SnaBI, SpeI, SphI, SspI, StuT, StyD41, StyL SwaI, T, Taqga TfiI, TliI, TseI, Tsp45L, Tsp509I, TspMI, TspRI, Tthl11, XbaI, XcmiI, XhoI, XmaI, XmnI, or ZraI.

In an aspect, a disclosed restriction enzyme can comprise a 4 bp cutter. In an aspect, a disclosed 4 base cutter can comprise AciI, AluI, BfaI, BfuCI, BstUI, CviAII, CviKI-1, CviQI, DpnI, DpnII, FatI, HaeIII, HhaI, HinPII, HpaII, HpyCH4IV, HpyCH4V, LpnPI, MboI, MluCI, MnlI, MseI, MspI, MspJT, NIaIlI, PhoI, RsaI, Sau3AI, TagαI, Tsp509T, AccII, AfaT, AluBL AoxI, AspLE, BscFI, Bshl2361, BshFI, Bshi, BsiSI, BsnL Bspl43I, BspACI, BspANI, Bsp NiI, BssMI, BstENiI, BstFNI, BstHHL BstKTI, BstMBIL BsuRI, CfoI, Csp6I, CviJI, CviRI, CviTL Fae, PaiI, FnuDiI, FspBI, GlaI, HapiI, HinITl, R9529, Hin6I, HpySE526T, Hsp92IL HspAI, Kzo9I, MacI, MaelI, MalI, MvnI, NdelH, PalI, RsaN1, SaqAI, SetI, SgeI, SgrTI, Sse91, SsiI, Sthl32I, TaiI, TaqI, TasI, ThaI, TrulI, Tru9I, TscI, TspEI, TthHB81, and XspI. In an aspect, a 4 bp cutter can provide better data resolution than, for example, a 6 bp cutter or a 8 bp cutter.

In an aspect, a first disclosed restriction enzyme can be CviQI. In an aspect, a second disclosed restriction enzyme can be NIaIII. In an aspect, a third disclosed restriction enzyme can be PmeI. In an aspect, a first disclosed restriction enzyme can be CviQI, a second disclosed restriction enzyme can be NIaIII, and a third disclosed restriction enzyme can be PmeI. In an aspect, a disclosed method can use any combination of 4 bp cutters.

In an aspect, a disclosed population of cells can be crosslinked prior to incubating step of a disclosed method. Crosslinking is known to the art and crosslinking cells to preserve protein-chromatin interactions is also known to the art. Further, crosslinking protocols are also known to the art (see, e.g., Tian B, et al. (2012) Methods Mol. Biol. 809:105-120). In an aspect, a disclosed crosslinking protocol can comprise washing the population of cells with PBS, contacting the cells with accutase, removing the accutase, resuspending the cells with Dulbecco's Modified Eagle Medium (DMEM), contacting the cells with fixative agent, contacting the cells with glycine, pelleting the crosslinked cells by centrifugation, and washing the pelleted crosslinked cells using PBS.

In an aspect, a disclosed fixative agent can comprise formaldehyde, glutaraldehyde, ethanol-based fixatives, methanol-based fixatives, acetone, acetic acid, osmium tetraoxide, potassium dichromate, chromic acid, potassium permanganate. mercurials, picrates, formalin, paraformaldehyde, amine-reactive NHS-ester crosslinkers such as bis[sulfosuccinimidyl] suberate (BS3), 3,3′-dithiobis(sulfosuccinimidylpropionate] (DTSSP), ethylene glycol bis[sulfosuccinimidylsuccinate (sulfo-EGS), disuccinimidyl glutarate (DSG), disuccinimidyl suberate, dithiobis[succinimidyl propionate] (DSP), disuccinimidyl subcrate (DSS), ethylene glycol bis[succinimidylsuccinate] (EGS), NHS-ester/diazirine crosslinkers such as NHS-diazirine, NHS-LC-diazirine, NHS-SS-diazirine, sulfo-NI-IS-diazirine, sulfo-NHS-LC-diazirine. acrolein, glyoxal, carbodiimides, diimidoesters, choro-s-triazides, mercuric chloride, and sulfo-NHS-SS-diazirine. In an aspect, a population of cells can be fixed with formaldehyde. In an aspect, a disclosed fixative agent can comprise formaldehyde.

In an aspect, the isolating step of a disclosed method can comprise incubating the cells in a buffer comprising bovine serum albumin (BSA), dithiothreitol (DTT), and IGEPAL. In an aspect, the isolating step of a disclosed method can further comprise centrifuging the cells to isolate the nuclei and collecting the supernatant comprising cytoplasmic RNA.

In an aspect, the incubating step of a disclosed method can further comprise centrifuging the isolated nuclei to stop the reaction and collecting the supernatant comprising the nucleic RNA.

In an aspect, a disclosed Tn5 transposome can be a pre-assembled Tn5 transposome. In an aspect, a disclosed method can further comprise assembling the Tn5 transposome. In an aspect, assembling the Tn5 transposome can comprise annealing two Tn5 adaptors and incubating the annealed Tn5 adaptors with a Tn5 transposase. In an aspect, a disclosed Tn5 adaptor can comprise the sequence set forth in SEQ ID NO:01. In an aspect, a disclosed Tn5 adaptor can comprise the sequence set forth in SEQ ID NO:02. In an aspect, disclosed Tn5 adaptors used in a disclosed can comprise the sequence set forth in SEQ ID NO:01 and SEQ ID NO:02. In an aspect a disclosed Tn5 adaptor can comprise a Mosaic End sequence for Tn5 recognition and a single-stranded flanking sequence that ligates to CviQI-digested DNA fragment using a splint oligonucleotide. In an aspect, a skilled person can craft a Tn5 adaptor. In an aspect, a Tn5 adaptor for use in a disclosed method can comprise a ME sequence and a reverse complement sequence to the splint oligonucleotide and can have the ability to ligate to the restriction enzyme digested genomic DNA.

In an aspect, a disclosed splint oligonucleotide can comprise the sequence set forth in SEQ ID NO:03. In an aspect, the ligating in situ step of a disclosed method can comprise using a T4 DNA ligase and a ligation buffer (such as, for example, a T4 ligation buffer). In an aspect, a skilled person can craft a splint oligonucleotide. In an aspect, a splint oligonucleotide for use in a disclosed method can comprise a reverse complement sequence to the Tn5 adaptor. In an aspect. a disclosed splint oligonucleotide/Tn5 adaptor can have the ability to ligate to the restriction enzyme digested genomic DNA.

In an aspect, the reversing the crosslink step of a disclosed method can comprise resuspending the nuclei in Tris-HCL, Proteinase K, and NaCl. In an aspect, the purifying the reverse cross-linked DNA step of a disclosed method can comprise a phenol:chloroform:isoamyl alcohol treatment followed by ethanol precipitation.

In an aspect, a disclosed method can further comprise repairing the Tn5 transposition gap. In an aspect, repairing the Tn5 transposition gap can comprise incubating the purified DNA with dNTPs and a DNA polymerase (such as, for example, a T4 DNA polymerase). DNA polymerases are known in the art. In an aspect, a DNA polymerase can comprise DNA-dependent DNA polymerase activity, RNA-dependent DNA polymerase activity, or DNA-dependent and RNA-dependent DNA polymerase activity. In an aspect, DN A polymerases can be thermostable or non-thermostable. Example of DNA polymerases can include but are not limited to Taq polymerase, Tth polymerase. Tli polymerase, Pfu polymerase, Pfutubo polymerase, Pyrobest polymerase, Pwo polymerase, KOD polymerase, Bst polymerase, Sac polymerase, Sso polymerase, Poc polymerase. Pab polymerase, Mth polymerase, Pho polymerase. ES4 polymerase, VENT polymerase, DEEPVENT polymerase, EX-Tag polymerase, LA-Taq polymerase, Expand polymerases, Platinum Taq polymerases, Hi-Fi polymerase, Tbr polymerase, Tfl polymerase, Tru polymerase, Tac polymerase, Tne polymerase, Tma polymerase. Tih polymerase, Tfi polymerase, Kienow fragment, and variants, modified products and derivatives thereof.

In an aspect of a disclosed method, performing PCR can comprise mixing the digested purified DNA with dNTPs, a forward primer, a reverse primer, and a polymerase. In an aspect, a disclosed forward primer can have the sequence set forth in SEQ ID NO:04. In an aspect, a disclosed reverse primer can comprise the sequence set forth in SEQ ID NO:05. In an aspect, a skilled person can craft one or more primers for use in a disclosed method. In an aspect, a primer for use in a disclosed kit can amplify DNA from Tn5 inserted regions. In an aspect, a primer for use in a disclosed kit can amplify DNA ligated to Tn5 adaptor.

In an aspect of a disclosed method, the resulting amplified chimeric DNA fragment can contain one end derived from the CviQI digested genomic DNA and one end derived from the Tn5-tagmented open chromatin sequence. In an aspect of a disclosed method, the end derived from disclosed CviQI digested genomic DNA can be captured by Read 1 of each pair-end sequence. In an aspect of a disclosed method, the end derived from disclosed Tn5-tagmented open chromatin sequence can be captured by Read 2 of each pair-end sequence. In an aspect of a disclosed method, the end derived from disclosed CviQI digested genomic DNA can be captured by Read 1 of each pair-end sequence while the end derived from disclosed Tn5-tagmented open chromatin sequence can be captured by Read 2 of each pair-end sequence.

In an aspect, a disclosed method can further comprise using gel extraction to obtain those PCR products having a size of about 400-600 bp. Gel extraction techniques are known to the art. In an aspect, gel extracted PCR products can be subjected to deep sequencing. As known to the art, deep sequencing is synonymous with next generation sequencing and refers to sequencing a genomic region multiple times (e.g., sometimes hundreds or even thousands of times). Deep sequencing protocols are known to the art.

In an aspect, the creating a RNA-Seq library step of a disclosed method can comprise combining the supernatant comprising cytoplasmic RNA and the supernatant comprising nucleic RNA and reversing the crosslink. In an aspect, a disclosed method can further comprises purifying the reverse crosslinked RNA. In an aspect, a disclosed method can further comprise dissolving the purified RNA and treating the purified RNA with DNase to remove DNA in solution. In an aspect, a disclosed method can further comprise using a sample of the purified RNA to create a RNA-Seq library. RNA-Seq and RNA-Seq protocols are well-known to the art. In an aspect, the creating an RNA-Seq library in a disclosed method can comprise using a smartseq2 protocol.

In an aspect, a disclosed population of cells can comprise at least 75,000 cells, at least 80,000 cells, at least 85,000 cells, at least 90,000 cells, at least 95,000 cells, at least 100,000 cells, at least 105,000 cells, at least 110,000 cells, at least 115,000 cells, at least 120,000 cells, or at least 125,000 cells. In an aspect, a disclosed population of cells can comprise about 75,000 to about 125,000 cells or can comprise about 100,000 cells.

In an aspect, a disclosed population of cells can comprise cells obtained from a biosample and then subjected to a crosslinking protocol. Crosslinking protocols are known to the art. In an aspect of a disclosed method, a disclosed crosslinking protocol can comprise washing the cells obtained from the biosample with PBS, contacting the cells with a digestion agent (such as, for example, accutase, collagenase, liberase, trypsin, TrypLE, non-enzymatic cell dissociation solution (NECDS)), removing the digestion agent, resuspending the cells with Dulbecco's Modified Eagle Medium (DMEM), contacting the cells with fixative agent, contacting the cells with glycine, pelleting the crosslinked cells by centrifugation, and washing the pelleted crosslinked cells using PBS.

In an aspect, a disclosed population of cells can be obtained from any number of sources or samples. For example, a biosample comprising cells for use in a disclosed method can be obtained from a subject by any number of means known to the art, including by obtaining or harvesting bodily fluids (e.g., blood, tears, urine, CSF, serum, lymph, mucus, saliva, anal and vaginal secretions. perspiration, and semen), taking tissue (e.g., a biopsy, graft, etc.), and/or by collecting cells. In an aspect, a disclosed population of cells can comprise a single type of cell or multiple types of cells. In an aspect, a disclosed population of cells can be heterogenous or homogenous. A disclosed population of cells can comprise a singular type of organism or multiple types of organisms. In an aspect, a disclosed biosample can be obtained from a subject. In an aspect, a disclosed method can comprise obtaining a biosample from a subject. In an aspect, a disclosed method can comprise obtaining a population of cells from the subject's biosample. In an aspect, a disclosed biosample can comprise a low input clinical sample. In an aspect, a disclosed population of cells can comprise a low input clinical sample.

In an aspect, a subject can have been diagnosed with or can be suspected of having a disease or disorder. In an aspect, a disease or disorder can be a disease or disorder associated with chromatin deregulation and/or chromatin dysregulation. Diseases or disorder associated with chromatin deregulation and/or chromatin dysregulation are known to the art and include but are not limited to Alzheimer's disease. Amyotrophic lateral sclerosis (ALS), Angelman syndrome, ATR-X syndrome, Brachydactyly mental retardation syndrome, cerebro-oculo-facio-skeletal syndrome (COFS). Chromatin remodeling CHARGE syndrome, Cockayne syndrome, Coffin-Siris syndrome, Facioscapulohumera muscular dystrophy (FSHD), Fragile X syndrome, Huntington's disease. Immunodeficiency, centromeric region instability, and facial anomalies syndrome (ICF), Juberg-Marsidi syndrome, Kabuki syndrome, Kleefstra syndrome, MRD12, MRD14, MRD15, MRD16, Parkinson's disease. Prader-Willi syndrome, Rett syndrome, Rubinstein-Taybi syndrome, Smith-Fineman-Myers syndrome, Sotos syndrome, Sutherland-Haan syndrome, Weaver syndrome, and X-linked mental retardation.

In an aspect, a subject can be diagnosed with or can be suspected of having a disease or disorder affected by a gene having chromatin deregulation and/or chromatin dysregulation. Such diseases or disorders are known to the art and include but are not limited to 15q11-q13 locus, A2aR, APOE, ARID1A (BAF250A), ARID1B (BAF250B), ATRX (RAD54L), CHD7, CREBBP (CBP, KAT3A), DNMT3B, EHMT1 (GLP, KMT1D), EP300 (KAT3B), ERCC6 (CSB), EZH2 (KMT6), FMR1, FSHD locus 4q35, FUS (TLS), HDAC4, JARID1C (SMCX, KDM5C), MARCB1 (BAF47, SNF5LI), MECP2, MLL2 (KMT2B), NSD1 (KMT3B), PHF8. SCA7 locus, SMARCA2(BRM, BAF190B, SNF2A), SMARCA4 (BRG1, BAF190A, SNF2B), SNCA (alpha-synuclein), TNFA (TNF-alpha), UBE3A (E6AP), and UTX (KDM6A).

In an aspect, a subject can be diagnosed with or can be suspected of having a critical limb ischemia (CLI).

In an aspect, a disclosed method can comprise subjecting a disclosed population of cells to a crosslinking protocol.

In an aspect, a disclosed method of performing HiCAR can further comprise repeating one or more steps of the method using a second population of cells. In an aspect, a disclosed method can further comprise repeating all the steps of the method using a disclosed second population of cells. In an aspect, a disclosed second population of cells can comprise cells obtained from a disclosed second biosample and then subjected to a crosslinking protocol. In an aspect, a disclosed second population of cells can be obtained from any number of sources or samples. For example, a disclosed second biosample comprising cells for use in a disclosed method can be obtained from a subject by any number of means known to the art, including by obtaining or harvesting bodily fluids (e.g., blood, tears, urine, CSF, serum, lymph, mucus, saliva, anal and vaginal secretions, perspiration, and semen), taking tissue (e.g., a biopsy, graft, etc.), and/or by collecting cells. In an aspect, a disclosed second population of cells can comprise a single type of cell or multiple types of cells. In an aspect, a disclosed second population of cells can be heterogenous or homogenous. A disclosed second population of cells can comprise a singular type of organism or multiple types of organisms. In an aspect, a disclosed method can comprise obtaining a disclosed second biosample from a subject. In an aspect, a disclosed method can comprise obtaining a disclosed second population of cells from the subject's biosample. In an aspect, a disclosed biosample can comprise a low input clinical sample. In an aspect, a disclosed population of cells can comprise a low input clinical sample.

In an aspect, a disclosed second biosample can be obtained from a subject. In an aspect, a disclosed second biosample can be obtained from a subject not having been diagnosed with or not suspected of having a disease or disorder. In an aspect, a disclosed second biosample can be obtained from a subject having been diagnosed with or is suspected of having a disease or disorder. In an aspect, a disclosed second biosample can be obtained from the same subject that provided the disclosed first biosample. In an aspect, the first and second disclosed populations of cells can be obtained from the same subject. In an aspect, the first and second disclosed populations of cells can be obtained from different subjects. In an aspect, the first and second disclosed populations of cells can be obtained from the same subject, wherein the disclosed first population is obtained prior to a treatment and wherein the disclosed second population is obtained after the treatment.

In an aspect, a disclosed method of performing HiCAR can comprise repeating one or more steps of the method using additional populations of cells (e.g., a third population, a fourth population, a fifth population, etc.). In an aspect, a disclosed method can be repeated one or more times using a new population of cells each time the method is repeated. In an aspect, a disclosed method can be used to compare chromatin interactions and chromatic accessibility across multiple populations of cells (e.g., a first population, a second population, a third population, a fourth population, so forth and so on). In an aspect, a disclosed method can be used to compare RNA-Seq data across multiple populations of cells (e.g., a first population, a second population, a third population, a fourth population. so forth and so on). In an aspect, a disclosed method can be used to compare RNA-Seq data to a pre-existing database.

In an aspect, a disclosed population of cells can comprise cultured cells. In an aspect, a first disclosed population of cells can comprise cultured cells, a second disclosed population of cells can comprise cultured cells, or both a first disclosed population and a second disclosed population of cells can comprise cultured cells. In an aspect, a disclosed population of cultured cells can comprise wild-type. normal, non-diseased, and/or non-disordered cells. In an aspect, a disclosed population of cultured cells can comprise mutant, atypical, diseased, and/or disordered cells. In an aspect, disclosed cultured cells can be mESCs, GM12878 cells, and/or H1 hESCs.

In an aspect, a disclosed method can further comprise processing the resulting HiCAR datasets obtained from a disclosed second population, a disclosed third population, or any other disclosed population of cells. In an aspect, processing the HiCAR datasets obtained from any other disclosed population of cells can comprise mapping and visualizing the uniquely mapped paired-end tags using a bioinformatics software program for visualizing molecular interactions, generating a comprehensive map of cis-regulatory chromatin contacts, calculating a cumulative interactive score for each HiCAR interaction anchor, or any combination thereof. In an aspect, a disclosed method can identify chromatin interactions that are enriched across multiple chromatin states. In an aspect, multiple chromatin states can comprise enhancers, promoters, and regions associated with active, poised, bivalent, and repressed chromatin states.

In an aspect, a disclosed method can comprise comparing HiCAR datasets obtained from the first population of cells to the HiCAR datasets obtained from the second population of cells. In an aspect, a disclosed method can comprise comparing HiCAR datasets obtained from multiple populations of cells. In an aspect, a disclosed method can comprise comparing a HiCAR dataset obtained from a first population to a HiCAR dataset obtained from multiple population of cells (e.g., a second population, a third population, a fourth population, a fifth population, etc.).

In an aspect, a disclosed method can further comprise identifying transcriptome differences between the two or more, three or more, four or more, five or more, or more than five populations of cells.

In an aspect, a disclosed method can further comprise identifying differences in cis-regulatory chromatin interactions between two or more, three or more, four or more, five or more, or more than five populations of cells. In an aspect, a disclosed method can further comprise identifying differences in chromatin accessibility between two or more, three or more, four or more, five or more, or more than five populations of cells.

In an aspect, a disclosed method can generate about 10-fold to about 20-fold more cis-paired-end tags than Trac-looping or can generate about 15-fold to about 18-fold more cis-paired-end tags than Trac-looping.

In an aspect, a disclosed method can generate greater than 200 million pair-end raw reads. or about 250 million to about 350 million pair-end raw reads, or about 300 million pair-end raw reads, or greater than 300 million pair-end raw reads. In an aspect, a disclosed method can generate about 100 million to about 200 million uniquely mapped paired-end tags, or more than 100 million uniquely mapped paired-end tags, or more than 200 million uniquely mapped paired-end tags.

In an aspect of a disclosed method, the resolution of the cis-regulatory chromatin contacts can comprise about 5 KB, about 10 KB, about 15 KB, about 20 KB, or greater than 20 KB. In an aspect of a disclosed method, the resolution of the cis-regulatory chromatin contacts can comprise about 5 KB.

In an aspect, a disclosed method can capture “active-to-active” interactions and/or “inactive-to-inactive” interactions in one or more populations of cells. In an aspect, a disclosed method can capture the number of active-to-active interactions to the number of inactive-to-inactive interactions in one or more populations of cells. In an aspect, a disclosed method can further comprise comparing the interaction strength/confidence of the active-to-active interactions to interaction strength/confidence of the inactive-to-inactive interactions in one or more populations of cells. In an aspect, a disclosed method can further comprise comparing the transcriptional/enhancer activity of the active-to-active interactions to the transcriptional/enhancer activity of the inactive-to-inactive interactions in one or more populations of cells.

In an aspect, a disclosed Tn5 transposome can be a pre-assembled Tn5 transposome. In an aspect, a disclosed method can further comprise assembling a Tn5 transposome prior to a disclosed incubating step. In an aspect, assembling a disclosed Tn5 transposome can comprise annealing a first Tn5 adaptor and a second Tn5 adaptor and mixing the annealed Tn5 adaptor with Tn5 transposase. In an aspect, a disclosed method can further comprise purifying Tn5 transposase from transformed bacteria carrying a Tn5, expression plasmid.

In an aspect, a disclosed method can further comprise integrating public epigenome datasets into a disclosed processing step.

In an aspect of a disclosed method, processing HiCAR datasets can comprise using a distiller pipeline. Distiller pipelines are known to the art. For example, in an aspect, a disclosed method can comprise using a distiller pipeline found at https://github.com/mirnylab.distiller-nf. In an aspect, processing HiCAR datasets can comprise one or more of the following: aligning the reads to hg38 reference genome using bwa mem with flags -SP; parsing the alignments: generating paired end tags (PET) using the pairtools (e.g., https://github.com/mirnylab/pairtools); filtering out PETs with low mapping quality (MAPQ <10); removing PETs with the same coordinate on the genome or mapped to the same digestion fragment; flipping uniquely mapped PETs as side 1 with the lower genomic coordinate; aggregating the flipped uniquely mapped PETs into contact matrices in the cooler format using the cooler tools at delimited resolution; extracting dense matrix data from cooler files; and visualizing the dense matrix data using HiGlass. In an aspect, a disclosed method can further comprise calculating the R1 and R2 reads signal around TSS or peaks prior to PET flipping.

In an aspect of a disclosed method, the similarity between different Hi-C datasets can be measured by HiCRep (described by Yang T, et al. (2017) Genome Res. 27:1939-1949). In an aspect, the stratum adjusted correlation coefficient (SCC) can be calculated on a per chromosome basis using HiCRep on 100 KB resolution data with a max distance of 5 Mb. In an aspect, the SCC can be calculated as a weighted average of stratum-specific Pearson's correlation coefficients.

In an aspect of a disclosed method, compartmentalization, directionality index and insulation score can be assessed using cooltools (see https://github.com-mirnylab/cooltools). Briefly, eigenvector decomposition can be performed on cis contact maps at 100 KB resolution. The first three eigenvectors and eigenvalues can be calculated, and the eigenvector associated with the largest absolute eigenvalue can be chosen. An identically binned track of GC content can be used to orient the eigenvectors. The insulation score and directionality index can be computed by cooltools using ‘find_insulating_boundaries’ and ‘directionality’ function, respectively.

In an aspect of a disclosed method, the curves of contact probability as a function of genomic separation can be generated by pairsqc following the 4DN pipeline (see https://github.com/4dn-dcic/pairsqc). Briefly, the genome can be binned at log 10 scale at interval of 0.1. For each bin, contact probability can be computed as number of reads/number of possible reads/bin size.

To process the HiCAR RNA profile data, reads can be aligned to hg38 genome with Hisat2 (Kim D. et al. (2019) Nat. Biotechnol. 37:907-915) using hg38 genome_tran index obtained from Hisat2 website (http://daehwankimlab.github.io/hisat2/download/). Raw reads for each gene can be quantified using featureCounts.

To process HiCAR 1D open chromatin peak in a disclosed method, unique mapped HiCAR DNA library R2 reads can be extracted before PET flipping. R2 reads from long range (>20 KB) and the inter-chromosome tans-PETs can be combined and processed to be compatible as MACS2 input BED files. R2 reads from the short-range cis-PETs can be discarded to avoid the potential bias due to proximity to CviQI enzyme cut sites (Lareau C A, et al. (2018) Nature Methods. 15:155-156). MACS2 can be used to identify ATAC peaks following the ENCODE pipeline (see https://github.com/ENCODE-DCC/atac-seq-pipeline) with the following parameters: “-q 0.01 --shift 150 --extsize -75-nomodel -B --SPMR --keep-dup all”.

In an aspect of a disclosed method, a CTCF ChIP-seq peak list of H1 can be downloaded from ENCODE (accession No. ENCFF82IAQO) and searched for CTCF sequence motifs using gimme (Van Heeringen S J, et al. (2011) Bioinformatics. 27:270-271) and CTCF motif (MA0139.1) from the JASPAR database (Fornes O, et al. (2020) Nucleic Acid Res. 48:D87-D92). In an aspect of a disclosed method, a subset of interactions with both ends containing either a single CTCF motif or multiple CTCF motifs in the same direction can be selected. In an aspect. the frequency of all possible directionality of CTCF motif pairs, convergent, tandem and divergent can be evaluated.

In an aspect, a disclosed method can comprise chromatin interaction calling. In an aspect, HiCAR, PLAC-seq, and HiChIP datasets can be used. In an aspect, a disclosed method can use MAPS to call the significant chromatin interactions. In an aspect, paired-end tags can first be extracted from cooler datasets at 5 KB or 10 KB resolution using the “cooler dump” function with parameters: “-t pixels -H -join”. In an aspect, interaction anchor bins can be defined by the ATAC peaks or corresponding ChIP-seq peaks called using MACS2. MAPS can apply a positive Poisson regression-based approach to normalize systematic biases from restriction enzyme cut sites, GC content, sequence mappability, and ID signal enrichment. In an aspect, interactions that were located within 15 KB of each other at both ends into clusters can be grouped and all other interactions can be classified as singletons. In an aspect, interactions with 6 or more and normalized contact frequency (raw read counts/expected read counts) >=2 can be retained and the significant interactions can be defined by FDR <0.01 for clusters and FDR <0.0001 for singletons. In an aspect of a disclosed method that addresses the situ Hi-C dataset, the .hic file can be downloaded from 4DN data portal (accession No. 4DNES2M5JIGV) and HiCCUPS can be applied to call interactions at 10 KB resolution with the following parameters: “-r 10000 -k KR -f 0.1,.1 -p 4,2 -i 7.5 -t 0.02,1.5,1.75,2 -d 20000,20000”.

In an aspect of a disclosed method, chromatin state calls can be obtained from the Roadmap Epigenomics Mapping Consortium. In an aspect, chromatin state calls can comprise a 18-state model. To determine which pairs of chromatin states are enriched at interaction anchors at a statistically significant level, the distribution of chromatin states can be examined at interaction anchors using HOMER. In an aspect. it can be assessed whether a connection between the feature is over-represented or under-represented given the general enrichment for each chromatin states at the interaction anchors. In an aspect, the HOMER “annotateInteractions” function can be used to obtain the p value and enrichment fold ratio for all pairs of chromatin states. The FDR adjusted p values can be obtained using the p.adjust function from the R package, with option method=“fdr”.

In an aspect, the enrichment for HiCAR identified interactions in significant eQTL-TSS association can be tested. In an aspect, the eQTL-TSS associations can be obtained. To assess the significance of the enrichment, in an aspect, a null distribution can be generated by creating a simulated interaction datasets by resampling the same number of interactions at random from distance-matched interactions (with 10,000 repeats). In an aspect, the empirical P-value can be computed by comparing the observed overlapping number with the null distribution.

In an aspect of a disclosed method, epigenetic features can be collected from a public database or consortium (e.g., the ENCODE consortium). In an aspect, average bigWig signals on each 5 KB anchor can be computed using the bigWigAverageOverBed command from UCSC. In an aspect, regression-based machine learning can be employed in a disclosed method. For regression, in an aspect, a sigmoid function can be used to scale the chromatin interaction score into a [0,1] range:

$$f ( x ) = \frac{1}{1 + {e}^{- c 1 ( x - c 2 )}}$$

In an aspect, c1 can be set to 0.05 and c2 can be set to 20 empirically, such that the bins with stronger interactions can have a value closer to 1 after sigmoid conversion. In an aspect, regression methods in the scikit-learn Python package can be used for regression analysis, including linear regression. decision tree, xbgboost. random forest and linear-kernel support vector machine (SVM). In an aspect, the XGBoost Python package can be used for XGBoost regression analysis.

In an aspect, a disclosed method can comprise a gene ontology (GO) enrichment analysis. In an aspect. Clusterprofile can be used to examine whether particular gene sets are enriched in certain gene lists. In an aspect, GO categories with “BH” adjusted p value <0.05 can be considered significant.

### D. Methods of Performing a Genome-Wide Profiling of Chromatin Interactions and/or Accessibility and Gene Expression

Disclosed herein is a method of performing a genome-wide profiling of chromatin interactions and/or accessibility and gene expression, the method comprising performing PCR using purified and tagmented DNA; and creating an RNA-Seq library using cytoplasmic and nucleic RNA, wherein the steps are performed using the same population of cells.

In an aspect of a disclosed method of performing a genome-wide profiling of chromatin interactions and/or accessibility and gene expression, purifying and tagmenting DNA can comprise one or more of the following: isolating nuclei from a population of cells; incubating the isolated nuclei with an assembled Tn5 transposome; digesting the isolated nuclei with a first restriction enzyme; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with a second restriction enzyme; circularizing the digested DNA and purifying the circularized DNA: digesting the purified DNA with a third restriction enzyme, or any combination thereof. In an aspect of a disclosed method of performing a genome-wide profiling of chromatin interactions and/or accessibility and gene expression, purifying and tagmenting DNA can comprise isolating nuclei from a population of cells; incubating the isolated nuclei with an assembled Tn5 transposome; digesting the isolated nuclei with a first restriction enzyme; incubating the digested nuclei with a splint oligonucleotide: ligating in situ the Tn5 adaptors to the proximal genomic DNA; reversing the crosslink: purifying the reverse cross-linked DNA and dissolving the purified DNA: digesting the purified DNA with a second restriction enzyme; circularizing the digested DNA and purifying the circularized DNA; and digesting the purified DNA with a third restriction enzyme. In an aspect, the steps in a disclosed method can be performed in the order as listed.

In an aspect, a disclosed method of performing a genome-wide profiling of chromatin interactions and/or accessibility and gene expression can identify cis-regulatory chromatin interactions and can characterize chromatin accessibility.

In an aspect, creating a RNA-Seq library can comprise one or more of the following: combining supernatant comprising cytoplasmic RNA and supernatant comprising nucleic RNA; reversing the crosslink; purifying the reverse crosslinked RNA; dissolving the purified RNA; treating the purified RNA with DNase: or any combination thereof. In an aspect, creating a RNA-Seq library can comprise combining supernatant comprising cytoplasmic RNA and supernatant comprising nucleic RNA; reversing the crosslink; purifying the reverse crosslinked RNA; dissolving the purified RNA: treating the purified RNA with DNase; and creating an RNA-Seq library. In an aspect, creating an RNA-Seq library can comprise using a smartseq2 protocol. In an aspect, the steps of a disclosed method of analyzing the transcriptome can be performed in the order as listed.

In an aspect, a disclosed method of performing a genome-wide profiling of chromatin interactions and/or accessibility and gene expression can further comprise processing the resulting datasets. In an aspect, processing the resulting datasets can comprise mapping and visualizing the uniquely mapped paired-end tags using a bioinformatics software program for visualizing molecular interactions, generating a comprehensive map of cis-regulatory chromatin contacts. calculating a cumulative interactive score for each interaction anchor, or any combination thereof. In an aspect, a disclosed method can identify chromatin interactions that are enriched across multiple chromatin states. In an aspect, multiple chromatin states can comprise enhancers, promoters, and regions associated with active, poised, bivalent, and repressed chromatin states.

In an aspect, a disclosed restriction enzyme can comprise a restriction site of 1, 2, 3, 4, 5, 6, or 8 bases long. In an aspect of a disclosed method performing a multi-omics assay, the first, second, and third restriction enzymes are the same. In an aspect of a disclosed method, the first, second, and third restriction enzymes are different. In an aspect of a disclosed method, two of the first, second, and third restriction enzymes are the same. Restriction enzymes suitable for a disclosed method performing a multi-omics assay are disclosed infra. In an aspect, a disclosed restriction enzyme can comprise a 4 bp cutter. 4 bp cutters suitable for a disclosed method performing a multi-omics assay are disclosed infra. In an aspect, a 4 bp cutter can provide better data resolution than, for example, a 6 bp cutter or a 8 bp cutter. In an aspect, a first disclosed restriction enzyme can be CviQI. In an aspect, a second disclosed restriction enzyme can be NIaIII. In an aspect, a third disclosed restriction enzyme can be PmeI. In an aspect, a disclosed first restriction enzyme can be CviQI, the second restriction enzyme can be NIaIII, and the third restriction enzyme can be PmeI. In an aspect, a disclosed method can use any combination of 4 bp cutters.

In an aspect, a disclosed population of cells can be cross-linked. Crosslinking is known to the art and crosslinking cells to preserve protein-chromatin interactions is also known to the art. Further, crosslinking protocols are also known to the art and are discussed supra. Fixative agents suitable for use in a disclosed method are disclosed supra.

In an aspect, a disclosed isolating step can further comprise centrifuging the cells to isolate the nuclei and collecting the supernatant comprising cytoplasmic RNA. In an aspect, a disclosed incubating step can further comprise centrifuging the isolated nuclei and collecting the supernatant comprising the nucleic RNA.

In an aspect, a disclosed method can comprise assembling the Tn5 transposome. In an aspect, assembling a disclosed Tn5 transposome can comprise annealing two Tn5 adaptors and incubating the annealed Tn5 adaptors with a Tn5 transposase. In an aspect, a disclosed Tn5 adaptor can comprise the sequence set forth in SEQ ID NO:0l and the other Tn5 adaptor can comprise the sequence set forth in SEQ ID NO:02. In an aspect, a skilled person can craft a Tn5 adaptor. In an aspect, a Tn5 adaptor for use in a disclosed method can comprise a ME sequence and a reverse complement sequence to the splint oligonucleotide and can have the ability to ligate to the restriction enzyme digested genomic DNA. In an aspect of a disclosed method of performing a multi-omics assay, a disclosed splint oligonucleotide can comprise the sequence set forth in SEQ ID NO:03. In an aspect, the ligating in situ step of a disclosed method can comprise using a T4 DNA ligase and a ligation buffer (such as, for example, a T4 ligation buffer). In an aspect, a skilled person can craft a splint oligonucleotide. In an aspect, a splint oligonucleotide for use in a disclosed method can comprise a reverse complement sequence to the Tn5 adaptor. In an aspect, a disclosed splint oligonucleotide/Tn5 adaptor can have the ability to ligate to the restriction enzyme digested genomic DNA.

In an aspect, the reversing the crosslink step of a disclosed method can comprise resuspending the nuclei in Tris-HCL, Proteinase K, and NaCl. In an aspect, the purifying the reverse cross-linked DNA step of a disclosed method can comprise a phenol:chloroform:isoamyl alcohol treatment followed by ethanol precipitation.

In an aspect, a disclosed method can further comprise repairing the Tn5 transposition gap. In an aspect, repairing the Tn5 transposition gap can comprise incubating the purified DNA with dNTPs and a DNA polymerase (such as, for example, a T4 DNA polymerase). DNA polymerases are known to the art and disclosed supra.

In an aspect of a disclosed method of performing a genome-wide profiling of chromatin interactions and/or accessibility and gene expression, the performing PCR step can comprise mixing the digested purified DNA with dNTPs, a forward primer, a reverse primer, and a polymerase. In an aspect, a disclosed forward primer can comprise the sequence set forth in SEQ ID NO:04 and wherein the reverse primer can comprise the sequence set forth in SEQ ID NO:05. In an aspect, a skilled person can craft one or more primers for use in a disclosed method. In an aspect, a primer for use in a disclosed kit can amplify DNA from Tn5 inserted regions. In an aspect, a primer for use in a disclosed kit can amplify DNA ligated to Tn5 adaptor.

In an aspect of a disclosed method of performing a genome-wide profiling of chromatin interactions and/or accessibility and gene expression, the resulting amplified chimeric DNA fragment can contain one end derived from the CviQI digested genomic DNA and one end derived from the Tn5-tagmented open chromatin sequence. In an aspect, the end derived from the CviQI digested genomic DNA can be captured by Read 1 of each pair-end sequence and the end derived from the Tn5-tagmented open chromatin sequence can captured by Read 2 of each pair-end sequence.

In an aspect, a disclosed method of performing a genome-wide profiling of chromatin interactions and/or accessibility and gene expression can comprise using gel extraction to obtain those PCR products having a size of about 400-600 bp. In an aspect, the gel extracted PCR products can be subjected to deep sequencing. Deep sequencing protocols are known to the art.

In an aspect, a disclosed method does not comprise (or can exclude) antibody-mediated immunoprecipitation, adaptor ligation, biotin pulldown, or any combination thereof.

In an aspect, a disclosed population of cells can comprise at least 75,000 cells, at least 80,000 cells, at least 85,000 cells, at least 90,000 cells, a t least 95,000 cells, at least 100,000 cells, at least 105,000 cells, at least 110,000 cells, at least 115,000 cells, at least 120,000 cells, or at least 125,000 cells. In an aspect, a disclosed population of cells can comprise about 75,000 to about 125,000 cells or can comprise about 100,000 cells.

In an aspect, a disclosed population of cells can comprise cells obtained from a biosample and then subjected to a crosslinking protocol. Crosslinking protocols are known to the art and discussed supra.

In an aspect, a disclosed population of cells can be obtained from any number of sources or samples. For example, a disclosed biosample comprising cells for use in a disclosed method can be obtained from a subject by any number of means known to the art, including by obtaining or harvesting bodily fluids (e.g., blood, tears, urine, CSF, serum, lymph, mucus, saliva, anal and vaginal secretions. perspiration, and semen), taking tissue (e.g., a biopsy, graft, etc.), and/or by collecting cells. In an aspect, a disclosed population of cells can comprise a single type of cell or multiple types of cells. In an aspect, a disclosed population of cells can be heterogenous or homogenous. A disclosed population of cells can comprise a singular type of organism or multiple types of organisms. In an aspect, a disclosed biosample can be obtained from a subject. In an aspect, a disclosed method can comprise obtaining a disclosed biosample from a subject. In an aspect, a disclosed method can comprise obtaining a population of cells from the subject's biosample. In an aspect, a disclosed biosample can comprise a low input clinical sample. In an aspect, a disclosed population of cells can comprise a low input clinical sample.

In an aspect, a subject can be diagnosed with or can be suspected of having a disease or disorder. In an aspect, a disease or disorder can be a disease or disorder associated with chromatin deregulation and/or chromatin dysregulation. Diseases or disorder associated with chromatin deregulation and/or chromatin dysregulation are known to the art and discussed supra. In an aspect, a subject can be diagnosed with or can be suspected of having a disease or disorder affected by gene having chromatin deregulation and/or chromatin dysregulation. Such diseases or disorders are known to the art and are discussed supra. In an aspect, a subject can be diagnosed with or can be suspected of having a critical limb ischemia (CL).

In an aspect, a disclosed method can comprise repeating the steps using a second population of cells. In an aspect, a disclosed second population of cells can comprise cells obtained from a disclosed second biosample and then can then be subjected to a crosslinking protocol. In an aspect, a disclosed second biosample can be obtained from a subject. In an aspect, a disclosed biosample can be obtained from a subject not having been diagnosed with or not suspected of having a disease or disorder.

In an aspect of a disclosed method can further comprise processing the resulting datasets. In an aspect, a disclosed method can further comprise comparing the datasets obtained from the first population of cells to the datasets obtained from the second population of cells. In an aspect, a disclosed method can comprise measuring differences in the cis-regulatory chromatin interactions, the chromatin accessibility, the transcriptome, or any combination thereof between the two populations of cells.

In an aspect, a disclosed method of performing a multi-omics assay can generate about 10-fold to about 20-fold more cis-paired-end tags than Trac-looping or can generate about 15-fold to about 18-fold more cis-paired-end tags than Trac-looping.

In an aspect, a disclosed method can generate greater than 200 million pair-end raw reads, or about 250 million to about 350 million pair-end raw reads, or about 300 million pair-end raw reads, or greater than 30) million pair-end raw reads. In an aspect, a disclosed method can generate about 100 million to about 200 million uniquely mapped paired-end tags, or more than 100 million uniquely mapped paired-end tags, or more than 200 million uniquely mapped paired-end tags.

In an aspect of a disclosed method, the resolution of the cis-regulatory chromatin contacts can comprise about 5 KB, about 10 KB, about 15 KB, about 20 KB, or greater than 20 KB. In an aspect of a disclosed method, the resolution of the cis-regulatory chromatin contacts can comprise about 5 KB.

In an aspect, a disclosed Tn5 transposome can be a pre-assembled Tn5 transposome. In an aspect, a disclosed method can further comprise assembling a Tn5 transposome prior to a disclosed incubating step. In an aspect, assembling a disclosed Tn5 transposome can comprise annealing a first Tn5 adaptor and a second Tn5 adaptor and mixing the annealed Tn5 adaptor with Tn5 transposase. In an aspect, a disclosed method can further comprise purifying Tn5 transposase from transformed bacteria carrying a Tn5 expression plasmid.

In an aspect, a disclosed method can further comprise integrating public epigenome datasets into a disclosed processing step.

In an aspect, processing the datasets for a disclosed second population of cells (or any populations of cells) can comprise mapping and visualizing the uniquely mapped paired-end tags using a bioinformatics software program for visualizing molecular interactions for a disclosed second population of cells, generating a comprehensive map of cis-regulatory chromatin contacts a disclosed second population of cells, or any combination thereof. For example, in an aspect, a disclosed method can capture the number of active-to-active interactions to the number of inactive-to-inactive interactions in one or more populations of cells, or comparing the interaction strength/confidence of the active-to-active interactions to interaction strength/confidence of the inactive-to-inactive interactions in one or more populations of cells, or comparing the transcriptional/enhancer activity of the active-to-active interactions to the transcriptional/enhancer activity of the inactive-to-inactive interactions in one or more populations of cells, or any combination thereof.

In an aspect, processing a disclosed HICAR dataset can comprise using a distiller pipeline. Distiller pipelines are known to the art and are discussed supra.

### E. Methods of Performing a Co-Assay

Disclosed herein is a method of performing a co-assay, the method comprising (i) purifying and tagmenting DNA: (ii) performing PCR using the DNA of step (i); (iii) collecting cytoplasmic and nucleic RNA during step (i); and (iv) creating an RNA-Seq library using the RNA of step (iii), wherein the method identifies cis-regulatory chromatin interactions, characterizes chromatin accessibility, and analyzes the transcriptome in a population of cells.

In an aspect of a disclosed method of performing a co-assay, purifying and tagmenting DNA can comprise one or more of the following: isolating nuclei from a population of cells; incubating the isolated nuclei with an assembled Tn5 transposome; digesting the isolated nuclei with a first restriction enzyme; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with a second restriction enzyme; circularizing the digested DNA and purifying the circularized DNA; digesting the purified DNA with a third restriction enzyme, or any combination thereof. In an aspect of a disclosed method of performing a co-assay, purifying and tagmenting DNA can comprise isolating nuclei from a population of cells; incubating the isolated nuclei with an assembled Tn5 transposome; digesting the isolated nuclei with a first restriction enzyme; incubating the digested nuclei with a splint oligonucleotide: ligating in situ the Tn5 adaptors to the proximal genomic DNA; reversing the crosslink; purifying the reverse cross-linked DNA and dissolving the purified DNA; digesting the purified DNA with a second restriction enzyme; circularizing the digested DNA and purifying the circularized DNA; digesting the purified DNA with a third restriction enzyme, or any combination thereof. In an aspect, the steps in a disclosed method can be performed in the order as listed.

In an aspect, a disclosed method can identify cis-regulatory chromatin interactions and can characterize chromatin accessibility. In an aspect, a disclosed method of performing a co-assay can comprise isolating nuclei from a population of cells; incubating the isolated nuclei with an assembled Tn5 transposome; digesting the isolated nuclei with a first restriction enzyme; incubating the digested nuclei with a splint oligonucleotide; ligating in situ the Tn5 adaptors to the proximal genomic DNA; reversing the crosslink; purifying the reverse cross-linked DNA and dissolving the purified DNA: digesting the purified DNA with a second restriction enzyme; circularizing the digested DNA and purifying the circularized DNA: digesting the purified DNA with a third restriction enzyme; performing PCR to generate DNA libraries, wherein the method identifies cis-regulatory chromatin interactions and characterizes chromatin accessibility. In an aspect, the steps in a disclosed method can be performed in the order as listed.

In an aspect, analyzing the transcriptome can comprise one or more of the following: combining supernatant comprising cytoplasmic RNA and supernatant comprising nucleic RNA; reversing the crosslink; purifying the reverse crosslinked RNA; dissolving the purified RNA; treating the purified RNA with DNase; creating an RN A-Seq library, or any combination thereof. In an aspect, analyzing the transcriptome can comprise combining supernatant comprising cytoplasmic RNA and supernatant comprising nucleic RNA; reversing the crosslink; purifying the reverse crosslinked RNA; dissolving the purified RNA; treating the purified RNA with DNase; and creating an RNA-Seq library. In an aspect, creating an RNA-Seq library can comprise using a smartseq2 protocol. In an aspect, the steps of a disclosed method of analyzing the transcriptome can be performed in the order as listed.

In an aspect, a disclosed method of performing a co-assay can further comprise processing the resulting HiCAR datasets. In an aspect, processing the HiCAR datasets can comprise mapping and visualizing the uniquely mapped paired-end tags using a bioinformatics software program for visualizing molecular interactions, generating a comprehensive map of cis-regulatory chromatin contacts, calculating a cumulative interactive score for each HiCAR interaction anchor, or any combination thereof. In an aspect, a disclosed method can identify chromatin interactions that are enriched across multiple chromatin states. In an aspect, multiple chromatin states can comprise enhancers, promoters, and regions associated with active, poised, bivalent, and repressed chromatin states.

In an aspect, a disclosed restriction enzyme can comprise a restriction site of 1, 2, 3, 4, 5, 6, or 8 bases long. In an aspect of a disclosed method performing a co-assay, the first, second, and third restriction enzymes are the same. In an aspect of a disclosed method, the first, second, and third restriction enzymes are different. In an aspect of a disclosed method, two of the first, second, and third restriction enzymes are the same. Restriction enzymes suitable for a disclosed method performing a multi-omics assay are disclosed infra. In an aspect, a disclosed restriction enzyme can comprise a 4 bp cutter. 4 bp cutters suitable for a disclosed method performing a multi-omics assay are disclosed infra. In an aspect, a 4 bp cutter can provide better data resolution than, for example, a 6 bp cutter or a 8 bp cutter. In an aspect, a first disclosed restriction enzyme can be CviQI. In an aspect, a second disclosed restriction enzyme can be NIaIII. In an aspect, a third disclosed restriction enzyme can be PmeI. In an aspect, a disclosed first restriction enzyme can be CviQI, the second restriction enzyme can be NIaIII, and the third restriction enzyme can be PmeI. In an aspect, a disclosed method can use any combination of 4 bp cutters.

In an aspect, a disclosed population of cells can be cross-linked prior. In an aspect, a disclosed isolating step can further comprise centrifuging the cells to isolate the nuclei and collecting the supernatant comprising cytoplasmic RNA. In an aspect, a disclosed incubating step can further comprise centrifuging the isolated nuclei and collecting the supernatant comprising the nucleic RNA.

In an aspect, a disclosed method can comprise assembling the Tn5 transposome. In an aspect, assembling a disclosed Tn5 transposome can comprise annealing two Tn5 adaptors and incubating the annealed Tn5 adaptors with a Tn5 transposase. In an aspect, a disclosed Tn5 adaptor can comprise the sequence set forth in SEQ ID NO:01 and the other Tn5 adaptor can comprise the sequence set forth in SEQ ID NO:02. In an aspect, a skilled person can craft a Tn5 adaptor. In an aspect, a Tn5 adaptor for use in a disclosed method can comprise a ME sequence and a reverse complement sequence to the splint oligonucleotide and can have the ability to ligate to the restriction enzyme digested genomic DNA.

In an aspect of a disclosed method of performing a multi-omics assay, a disclosed splint oligonucleotide can comprise the sequence set forth in SEQ ID NO:03. In an aspect, a skilled person can craft a splint oligonucleotide. In an aspect, a splint oligonucleotide for use in a disclosed method can comprise a reverse complement sequence to the Tn5 adaptor. In an aspect, a disclosed splint oligonucleotide/Tn5 adaptor can have the ability to ligate to the restriction enzyme digested genomic DNA.

In an aspect of a disclosed method of performing a co-assay, the performing PCR step can comprise mixing the digested purified DNA with dNTPs, a forward primer, a reverse primer, and a polymerase. In an aspect, a disclosed forward primer can comprise the sequence set forth in SEQ ID NO:04 and wherein the reverse primer can comprise the sequence set forth in SEQ ID NO:05. In an aspect, a skilled person can craft one or more primers for use in a disclosed method. In an aspect, a primer for use in a disclosed kit can amplify DNA from Tn5 inserted regions. In an aspect, a primer for use in a disclosed kit can amplify DNA ligated to Tn5 adaptor.

In an aspect of a disclosed method of performing a co-assay, the resulting amplified chimeric DNA fragment can contain one end derived from the CviQI digested genomic DNA and one end derived from the Tn5-tagmented open chromatin sequence. In an aspect, the end derived from the CviQI digested genomic DNA can be captured by Read 1 of each pair-end sequence and the end derived from the Tn5-tagmented open chromatin sequence can captured by Read 2 of each pair-end sequence

In an aspect, a disclosed method of performing a co-assay can comprise using gel extraction to obtain those PCR products having a size of about 400-600 bp. In an aspect, the gel extracted PCR products can be subjected to deep sequencing.

In an aspect, a disclosed method of performing a co-assay can exclude adaptor ligation and/or biotin pull down.

In an aspect, a disclosed population of cells can comprise at least 75,000 cells, at least 80,000 cells, at least 85,000 cells, at least 90,000 cells, at least 95,000 cells, at least 100,000 cells, at least 105,000 cells, at least 110,000 cells, at least 115,000 cells, at least 120,000 cells, or at least 125,000 cells. In an aspect, a disclosed population of cells can comprise about 75,000 to about 125,000 cells or can comprise about 100,000 cells.

In an aspect, a disclosed population of cells can be obtained from any number of sources or samples. For example, a disclosed biosample comprising cells for use in a disclosed method can be obtained from a subject by any number of means known to the art, including by obtaining or harvesting bodily fluids (e.g., blood, tears, urine, CSF, serum, lymph, mucus, saliva, anal and vaginal secretions, perspiration, and semen), taking tissue (e.g., a biopsy, graft, etc.), and/or by collecting cells. In an aspect, a disclosed population of cells can comprise a single type of cell or multiple types of cells. In an aspect, a disclosed population of cells can be heterogenous or homogenous. A disclosed population of cells can comprise a singular type of organism or multiple types of organisms. In an aspect, a disclosed biosample can be obtained from a subject. In an aspect, a disclosed method can comprise obtaining a disclosed biosample from a subject. In an aspect, a disclosed method can comprise obtaining a population of cells from the subject's biosample. In an aspect, a disclosed biosample can comprise a low input clinical sample. In an aspect, a disclosed population of cells can comprise a low input clinical sample.

In an aspect, a subject can be diagnosed with or can be suspected of having a disease or disorder. In an aspect, a disease or disorder can be a disease or disorder associated with chromatin deregulation and/or chromatin dysregulation. Diseases or disorder associated with chromatin deregulation and/or chromatin dysregulation are known to the art and discussed supra. In an aspect, a subject can be diagnosed with or can be suspected of having a disease or disorder having a gene affected by chromatin deregulation and/or chromatin dysregulation. Such diseases or disorders are known to the art and discussed supra. In an aspect, a subject can be diagnosed with or can be suspected of having a critical limb ischemia (CLI).

In an aspect, a disclosed population of cells can comprise cells obtained from a biosample and then subjected to a crosslinking protocol. Crosslinking protocols are known to the art and are discussed supra. Fixative agents are known to the art and discussed supra.

In an aspect, a disclosed method of performing a co-assay can comprise repeating the steps using a second population of cells. In an aspect, a disclosed second population of cells can comprise cells obtained from a disclosed second biosample and then can then be subjected to a crosslinking protocol. In an aspect, a disclosed second biosample can be obtained from a subject. In an aspect, a disclosed biosample can be obtained from a subject not having been diagnosed with or not suspected of having a disease or disorder.

In an aspect of a disclosed method of performing a co-assay can further comprise processing the resulting datasets. In an aspect, a disclosed method can further comprise comparing the resulting datasets obtained from the first population of cells to the resulting datasets obtained from the second population of cells. In an aspect, a disclosed method can measure differences in the cis-regulatory chromatin interactions, the chromatin accessibility, the transcriptome, or any combination thereof between the two populations of cells.

In an aspect, processing the datasets can comprise mapping and visualizing the uniquely mapped paired-end tags for the second population of cells using a bioinformatics software program for visualizing molecular interactions, generating a comprehensive map of cis-regulatory chromatin contacts for the second population of cells, or any combination thereof. In an aspect, a disclosed method of performing a multi-omics assay can capture “active-to-active” interactions and/or “inactive-to-inactive” interactions for a disclosed second population of cells.

In an aspect, processing a disclosed dataset can comprise using a distiller pipeline. Distiller pipelines are known to the art and are discussed infra.

### F. Kits

Disclosed herein is a kit comprising one or more components and/or reagents for use in a disclosed method of performing a multi-omics assay. Disclosed herein is a kit comprising one or more components and/or reagents for use in a disclosed method of performing a high-throughput chromosome conformation capture on accessible DNA and mRNA-Seq co-assay (HiCAR). Disclosed herein is a kit comprising one or more components and/or reagents for use in a disclosed method of genome-wide profiling of chromatin interactions and/or accessibility and gene expression. Disclosed herein is a kit comprising one or more components and/or reagents for use in a disclosed method of performing a co-assay. Disclosed herein is a kit comprising one or more components and/or reagents for use in a disclosed method of identifying chromatin interactions and assessing chromatin accessibility. Disclosed herein is a kit comprising one or more components and/or reagents for use in a disclosed method of sequencing RNA.

In an aspect, a disclosed kit can comprise the components and/or reagents necessary to perform one or more steps of a disclosed methods, such as, for example, isolating nuclei from a population of cells; incubating the isolated nuclei with an assembled Tn5 transposome; digesting the isolated nuclei with a first restriction enzyme; incubating the digested nuclei with a splint oligonucleotide; ligating in situ the Tn5 adaptors to the proximal genomic DNA; reversing the crosslink; purifying the reverse cross-linked DNA and dissolving the purified DNA; digesting the purified DNA with a second restriction enzyme; circularizing the digested DNA and purifying the circularized DNA; digesting the purified DNA with a third restriction enzyme: performing PCR to generate DNA libraries; deep sequencing the DNA; and creating a RNA-Seq library.

In an aspect, a disclosed kit can comprise one or more Tn5 adaptors such as, for example, an adaptor having the sequence set forth in SEQ ID NO:01 or SEQ ID NO:02 or a sequence having at least 85% identity to the sequence set forth in SEQ ID NO:01 or SEQ ID NO:02. In an aspect a disclosed kit can comprise a Tn5 adaptor comprising a Mosaic End sequence for Tn5 recognition and a single-stranded flanking sequence that ligates to CviQI-digested DNA fragment using a splint oligonucleotide. In an aspect, a skilled person can craft a Tn5 adaptor. In an aspect, a Tn5 adaptor for use in a disclosed kit can comprise a ME sequence and a reverse complement sequence to the splint oligonucleotide and can have the ability to ligate to the restriction enzyme digested genomic DNA. In an aspect, a disclosed kit can comprise a Tn5 transposase. In an aspect, a disclosed kit can comprise a Tn5 expression plasmid and/or bacteria transformed with a Tn5 expression plasmid.

In an aspect, a disclosed kit can comprise one or more disclosed restriction enzymes. In an aspect, a disclosed kit can comprise three disclosed restriction enzymes. In an aspect, a disclosed kit can comprise CviQI, NIaIII, and PmeI.

In an aspect, a disclosed kit can comprise one or more disclosed fixative agents. Fixative agents are known in the art and are discussed supra. In an aspect, a disclosed kit can comprise formaldehyde.

In an aspect, a disclosed kit can comprise one or more disclosed splint oligonucleotides such as, for example, an oligonucleotide having the sequence set forth in SEQ ID NO:03. In an aspect, a skilled person can craft a splint oligonucleotide. In an aspect, a splint oligonucleotide for use in a disclosed kit can comprise a reverse complement sequence to the Tn5 adaptor. In an aspect, a disclosed splint oligonucleotide Tn5 adaptor can have the ability to ligate to the restriction enzyme digested genomic DNA.

In an aspect, a disclosed kit can comprise a disclosed digestion agent such as, for example, accutase, collagenase, liberase, trypsin, TrypLE, non-enzymatic cell dissociation solution (NECDS), or any combination thereof. In an aspect, a disclosed kit can comprise accutase.

In an aspect, a disclosed kit can comprise one or more primers. In an aspect, a disclosed primer can have the sequence set forth in SEQ ID NO:04 or SEQ ID NO:05. In an aspect, a skilled person can craft one or more primers for use in a disclosed kit. In an aspect, a primer for use in a disclosed kit can amplify DNA from Tn5 inserted regions. In an aspect, a primer for use in a disclosed kit can amplify DNA ligated to Tn5 adaptor.

In an aspect, a disclosed kit can comprise one or more polymerases. Polymerases are known to the art and are discussed supra. In an aspect, a disclosed kit can comprise

In an aspect, a disclosed kit can comprise one or more ligases (such as, for example, a T4 DNA ligase). dNTPs, one or more DNA polymerases (such as, for example, a T4 DNA polymerase), one or more transposases (such as, for example, a Tn5 transposase), one or more transformed bacteria, or any combination thereof.

In an aspect, a disclosed kit can comprise at least two components and/or reagents constituting the kit. Together, the components and/or reagents constitute a functional unit for a given purpose (such as, for example, performing HiCAR or performing a multi-omics assay). Individual member components may be physically packaged together or separately. For example, a kit comprising an instruction for using the kit may or may not physically include the instruction with other individual member components and/or reagents. Instead, the instruction can be supplied as a separate member component and/or reagent, either in a paper form or an electronic form which may be supplied on computer readable memory device or downloaded from an internet website. or as recorded presentation. In an aspect, a kit for use in a disclosed method can comprise one or more containers holding a disclosed component and/or reagent and a label or package insert with instructions for use. In an aspect, suitable containers include, for example, bottles, vials, syringes, blister pack, etc. The containers can be formed from a variety of materials such as glass or plastic. The container can hold, for example, a disclosed component and/or reagent and can have a sterile access port (for example the container may be an intravenous solution bag or a vial having a stopper pierceable by a hypodermic injection needle). The label or package insert can indicate that a disclosed component and/or reagent can be used in a disclosed method. In an aspect, a disclosed kit can comprise additional components and/or reagents necessary for administration such as, for example, other buffers, polymerases, primers, chemical reagents, diluents, filters, needles, and syringes.

### VIII. EXAMPLES

### A. Introduction

As detailed in the specific examples that follow, HiCAR (High-throughput chromosome conformation capture on Accessible DNA with mRNA-Seq co-assay) is a novel method that enables simultaneous assessment of cis-regulatory chromatin interactions and chromatin accessibility as well as evaluation of the transcriptome, which represents the functional output of chromatin structure and accessibility. Unlike immunoprecipitation-based methods (e.g., HiChIP, PLAC-seq, and ChIA-PET), HiCAR does not require target-specific antibodies. Instead, by leveraging principles of in situ Hi-C. ATAC-seq, and SMART-seq2 methods, HiCAR requires only ˜100,00) cells as input and avoids many potentially nucleic acid loss-prone steps, such as adaptor ligation and biotin-pull down. With similar sequencing depth, HiCAR outperforms Trac-looping (Lai B. et al. (2018) Nat. Methods. 15:741-747) by generating ˜17-fold more (18.3% versus 1.1%) long-range (>20 KB) cis-paired-end tags (cis-PET), even when starting from 1,000-fold fewer cells (1×10⁵ versus 1×10⁸ million). As a multi-omics co-assay, HiCAR also yields high-quality chromatin accessibility and transcriptome data from the same low-input starting material.

The data provided below demonstrate that HiCAR is a robust and cost-effective multi-omics assay. which is broadly applicable for simultaneous analysis of genome architecture, chromatin accessibility, and the transcriptome using low-input samples.

### B. Materials and Methods

### 1. Cell Culture and Crosslink

Hi hESCs (WiCell, WA01) were cultured in Matrigel (Corning. 354230) coated plates with Stabilized feeder-free maintenance medium mTeSR™ Plus (STEMCELL, #05825). mTeSR™ Plus was changed every other day. For crosslinking, cells were washed once by PBS, then treated by accutase (biolegend, 4423201) for 10 mins at 37° C. After removing the accutase, cells were resuspended by DMEM. Formaldehyde was added to the final concentration of 1%, incubated at room temperature for 10 mins. Glycine was added to the final concentration of 0.2M, incubated at room temperature for 10 mins to quench formaldehyde. Fixed cells were pelleted by centrifugation for 5 min at 4° C. and washed with ice-cold PBS once.

### 2. Tn5 Purification

Briefly, Rosetta DE3 cells transformed with Tn5 expression plasmid pTXB1-Tn5 (Addgene #60240) were cultured in 500 mL LB and incubated at 16° C. overnight for protein induction. The bacteria were collected by centrifuge and resuspended by pre-cooled HEGX (40 mM Hepes-KOH pH 7.2, 1.6 M NaCl, 2 mM EDTA, 20% Glycerol, 0.4% Triton-X100, Roche Complete Protease Inhibitor), sonicated to release the protein. PEI (10% PEI, 4.44% HCl, 800 mM NaCl. 20 mM Hepes, 0.3 mM EDTA, 0.2% Triton X-100, pH 7.2) were then added to the lysate in dropwise to precipitate the E. coli DNA. The lysate was centrifuged, and supernatant was loaded to Chitin column (BIO-RAD, #7372522). The column was rotated at 4° C. for 2-3 hr then washed by HEGX buffer. 15 mL HEGX buffer containing 100 mM DTT was added to elute the protein. The column was incubated for another 24 hr at 4° C. The elution fraction was collected and concentrated to about 1 mL by Amicon Ultracel 30K (Millipore. #UFC903024), then dialyzed twice by 1 L dialysis buffer (100 HEPES-KOH pi 7.2, 0.2 M NaCl, 0.2 mM EDTA, 2 mM DTT, 0.2% Triton X-100. 20% glycerol) for 24 hr using dialysis membrane tube (Spectra, D1614-11). Then the protein was added 80% glycerol to a final concentration of 50%.

### 3. Tn5 Transposase Assembly

To assemble Tn5, 50 μL of 200 μM ME-rev and 50 μL of 200 μM BfaI-truseqR1-pmeI-nextera7 (Table 2) were annealed by the following program: 95° C. 5 min, cool to 14° C. with a slow ramp 1° C.; per min. The annealed adaptor was mixed with Tn5 Transposase in 1:1.5 molar ratio, the mixture was mixed by pipette and incubated at room temperature for 30 mins.

### 4. A Detailed HICAR Protocol

The first step of HiCAR was nuclei preparation and tagmentation. Here, 100,000 crosslinked cells were treated by 1 mL NPB (PBS containing 5% BSA, 1 mM DTT, 0.2% IGEPAL, Roche Complete Protease Inhibitor) at 4° C. for 15 min to isolate the nuclei. After centrifugation, the supernatant containing cytoplasm RNA was saved for future RNA-Seq analysis. The isolated nuclei were resuspended in 350 μL 2×TB buffer (66 mM Tris-AC pH 7.8, 132 mM K-AC, 20 mM Mg-AC, 32% DMF), 335 μL water and 15 μL assembled Tn5 transposome. The oligos used for Tn5 adaptors are listed in Table 2. Next, nuclei are rotated at 37° C. for 1.5 hrs. Then, 350 μL of 40 mM EDTA was added to stop the reaction. After washing the nuclei once by 0.075% BSA, the nuclei were treated by 32.5 μL water, 5 μL 10×NEBuffer3.1 (NEB, #B7203S), 12.5 μL 2% SDS at 62° C. for 10 mins. After centrifugation at 850 g for 5 min, the supernatant containing nuclei RNA was collected for future RNA-Seq library construction. The nuclei were resuspended in 100 μL H₂O, 14 μL 10×NEBuffer3.1, 25 μL 10% Triton X-100, and incubated at 37° C. for 15 min to quench SDS.

The second step in HiCAR was CviQI digestion and in situ ligation. Here, the nuclei were washed by 1 mL 1.1×NEBbuffer 3. 1, then treated by 90 μL 1.1×NEBuffer 3.1 containing 100 U CviQI (NEB, #R0639L) and 3 μL of 200 μM TruseqR1 oligo (Table 2) at room temperature for 1 hr. After digestion, 48 μL 10×T4 ligation buffer, 6 μL T4 DNA ligase (400 U/μL, NEB, #M0202S), 2.4 μL 20 mg/ml BSA (NEB, #B9000S), 40 μL 10% Triton X-100, 283.6 μL H₂O), into the reaction and rotated the nuclei at room temperature for 4 hr.

The third step in HiCAR was reverse crosslink and DNA purification. After centrifugation at 2000 g for 5 min, the supernatant was discarded. The nuclei were resuspended in 200 μL of 10 mM Tris-HCl (pH 8.0). 5 μL Proteinase K (Thermofisher, #AM2546), 10 μL 20% SDS, incubated at 60° C. for 30 min. Next. 22 μL 5M NaCl was added to the buffer and the nuclei were incubated at 68° C. for at least 1.5 hrs to reverse crosslink. The DNA was purified by Phenol:Chloroform:isoamyl Alcohol (25:24:1, v/v, SPECTRUM, #136112-00-0) treatment followed by ethanol precipitation. The DNA was dissolved by 21 μL 10 mM Tris-HCl (pH 8.0).

The fourth step is NIaIII digestion and circularization. The purified DNA was incubated with 4 μL 10 mM dNTP, 5 μL 10× Cutsmart buffer 1.5 μL T4 DNA polymerase (NEB, #M0203L) and 20.5 μL H-O at room temperature for 30 min to repair the Tn5 transposition gap. Next, the reaction was incubated at 75° C. for 20 min to inactivate T4 DNA polymerase. After that, 43 μL water, 5 μL 10× CutSmart buffer, and 2 μL NIaIII (NEB, #R0125L) were added into the sample followed by incubation at 37° C. for 1 hr. The digested DNA was purified by 0.9×(90 μL) volume SPRI beads (BECKMAN, #B23319), and dissolved in 80 μL 10 mM Tris-HCl (pH 8.0) buffer. Next, the DNA was diluted to 0.6 ng/μL and circulated in T4 Ligation Buffer by T4 DNA ligase (400 U/μL, NEB, #M0202S). The sample was mixed and incubated at room temperature for at least 2 hrs. The DNA was purified by DNA clean & concentrator kit (Zymo, #1D4013) and eluted in 20 μL water.

The fifth step in HiCAR is PmeI digestion and PCR. Here. 18 μL purified DNA was mixed with 2.1 μL 10× CutSmart buffer and 0.9 μL PmeI at 37° C. for 1 hr to digest DNA. Then, 20 μL 5×Q5 buffer, 2 μL 10 mM dNTP, 2 μL primer1 (Table 2) (10 μM Nextera-pcr-i7-10-L), 2 μL primer2 (Table 2) (10 μM NEB primer i501), 1 μL Q5 polymerase (NEB. #m0491L) and 73 μL water was added into the sample. The PCR library amplification was performed using the following program (step 1-72° C. for 5 min then 98° C. for 30 sec; step 2-98° C. for 10 sec. 59° C. for 30 sec, 72° C. for 45 sed, repeating step 2 for an additional 11 cycles; step 3-72° C. for 5 min and 4° C. forever). After PCR, the DNA product between 400-600 bp was purified by gel extraction using DNA recovery kit (Zymo, #D4002) for deep sequencing.

The sixth step of HiCAR was the construction of RNA libraries. The cytoplasmic and nuclei RNA fraction was combined. Then 20% SDS was added to the pooled RNA fraction to make the final concentration of SDS as 1%. The sample was mixed and incubated at 60° C. for 30 min. After incubation, 1.9 volume of 5 M NaCl was added to make the final concentration of NaCl 500 mM, and the sample was incubated at 68° C. for at least 1.5 hrs for reverse crosslinking. Next, the RNA was purified by Phenol:Chloroform:Isoamyl Alcohol (25:24:1, v/v, SPECTRUM. #136112-00-0) extraction and ethanol precipitation. The sample was dissolved in 21 μl. 10 mM Tris-HCl (pH 8.0). Then the sample was treated by 0.5 μL DNaseI at 37° C. for 30 min to remove DNA in solution. The RNA was purified by 2× volume of SPRI beads, dissolved RNA by 20 μL 10 mM Tris-HCl (pH 8.0). Then take out 2.3 μL RNA to make an RNA-Seq library using smartseq2 protocol (Picelli S, et al. (2014) Nat. Protoc. 9:171-181).

### 5. HICAR Data Processing

HiCAR datasets were processed following the distiller pipeline (https://github.com:mirnylab/distiller-nf). Briefly, reads were aligned to hg38 reference genome using bwa mem with flags -SP. Alignments were parsed, and paired end tags (PET) were generated using the pairtools (https://github.commimylab/pairtools). PET with low mapping quality (MAPQ <10) were filtered out. PET with the same coordinate on the genome or mapped to the same digestion fragment were removed. Uniquely mapped PETs were flipped as side 1 with the lower genomic coordinate and aggregated into contact matrices in the cooler format using the cooler tools (Abdennur N, et al. (2020) Bioinformatics. 36:311-316) at delimited resolution (5 KB, 10 KB, 50 KB, 100 KB, 250 KB, 500 KB. 1 MB, 25 MB. 50 MB. 100 MB). The dense matrix data were extracted from cooler files and visualized using HiGlass (Kerpedjiev P, et al. (2018) Genome Biol. 19:125). The R1 and R2 reads signal around TSS or peaks were calculated with Enriched Heatmap (Gu Z, et al. (2018) BMC Genomics. 19:234) before PET flipping.

### 6. Hi-C Matrix Correlation SCC (Stratum-Adjusted Correlation Coefficient)

The similarity between different Hi-C datasets were measured by HiCRep (Yang T, et al. (2017) Genome Res. 27:1939-1949). The stratum adjusted correlation coefficient (SCC) is calculated on a per chromosome basis using HiCRep on 100 KB resolution data with a max distance of 5 Mb. The SCC was calculated as a weighted average of stratum-specific Pearson's correlation coefficients.

### 7. Compartments A and B, Directionality, and Insulation Score

Compartmentalization, directionality index, and insulation score was assessed using cooltools (https://github.com/mirnylab/cooltools). Briefly, eigenvector decomposition was performed on cis contact maps at 100-KB resolution. The first three eigenvectors and eigenvalues were calculated, and the eigenvector associated with the largest absolute eigenvalue was chosen. An identically binned track of GC content was used to orient the eigenvectors. The insulation score and directionality Index were computed by cooltools using ‘find_insulating_boundaries’ and ‘directionality’ function, respectively.

### 8. Contact Probability Decaying Curve

The curves of contact probability as a function of genomic separation were generated by pairsqc following the 4DN pipeline (https://github.com-4dn-dcic/pairsqc). Briefly, the genome was binned at log 10 scale at interval of 0.1. For each bin, contact probability was computed as number of reads/number of possible reads/bin size.

### 9. HICAR RNA Profile Processing

Reads were aligned to hg38 genome with Hisat2 (Kim D, et al. (2019) Nat. Biotechnol. 37:907-915) using hg38 genome_tran index obtained from Hisat2 website (http://daehwankimlab.github.io/hisat2/download). Raw reads for each gene were quantified using featureCounts (Liao Y, et al. (2014) Bioinformatics. 30:923-930).

### 10. HICAR 1D Open Chromatin Peak Processing

Unique mapped HiCAR DNA library R2 reads were extracted before PET flipping. R2 reads from long range (>20 KB) and the inter-chromosome trans-PETs were combined and processed to be compatible as MACS2 (Zhang Y, et al. (2008) Genome Biol. 9:R137) input BED files. R2 reads from the short-range cis-PETs were discarded to avoid the potential bias due to proximity to CviQI enzyme cut sites (Lareau C A, et al. (2018) Nature Methods. 15:155-156) MACS2 was used to identify ATAC peaks following the ENCODE pipeline (https://github.com/ENCODE-DCC/atac-seq-pipeline) with the following parameters: “-q 0.01 --shift 150 --extsize -75--nomodel -B --SPMR --keep-dup all”.

### 11. CTCF Motif Orientation Analysis

CTCF ChIP-seq peak list of H1 was downloaded from ENCODE (accession No. ENCFF821AQO) and searched for CTCF sequence motifs using gimme (van Heeringen S J, et al. (2011) Bioinformatics. 27:270-271) and CTCF motif (MA0139.1) from the JASPAR database (Fornes O, et al. (2020) Nucleic Acids Res. 48:187-D92). A subset of interactions with both ends containing either a single CTCF motif or multiple CTCF motifs in the same direction was then selected. The frequency of all possible directionality of CTCF motif pairs, convergent, tandem and divergent, were evaluated.

### 12. Chromatin Interaction Calling

For HiCAR, PLAC-seq and HiChIP datasets, MAPS was used to call the significant chromatin interactions. First, paired-end tags were extracted from cooler datasets at 5 KB or 10 KB resolution using the “cooler dump” function with parameters: “-t pixels -H --join”. The interaction anchor bins were defined by the ATAC peaks or corresponding ChIP-seq peaks called using MACS2 (Zhang Y, et al. (2008) Genome Biol. 9:R137). MAPS applied a positive Poisson regression-based approach to normalize systematic biases from restriction enzyme cut sites, GC content, sequence mappability, and ID signal enrichment. Interactions that were located within 15 KB of each other at both ends into clusters and classified all other interactions as singletons. Only interactions with 6 or more were retained and normalized contact frequency (raw read counts/expected read counts)>2 and the significant interactions were defined by FDR <0.01 for clusters and FDR <0.0001 for singletons. For in situ Hi-C dataset, the .hic file is downloaded from 4DN data portal (accession No. 4DNES2M5JIGV) and HiCCUPS (Durand N C, et al. (2016) Cell Syst. 3:95-98) is applied to call interactions at 10 KB resolution with the following parameters: “-r 10000 -k KR -f 0.1,.1 -p 4,2 -i 7,5 -t 0.02.1.5,1.75.2 -d 20000,20000”.

### 13. Chromatin States Enrichment Analysis at Chromatin Interaction Anchors

Using an 18-state model, chromatin state calls for Ill cell line were obtained from the Roadmap Epigenomics Mapping Consortium. To determine which pairs of chromatin states were enriched at interaction anchors at a statistically significant level, the distribution of chromatin states at interaction anchors using HOMER were examined. Whether a connection between the feature was over-represented or under-represented given the general enrichment for each chromatin states at the interaction anchors was determined. The HOMER “annotateInteractions” function was used to obtain the p value and enrichment fold ratio for all pairs of chromatin states. The FDR adjusted p values were obtained using the p.adjust function from the R package, with option method=“fdr”.

14. Comparison Between eQTL-TSS Association and HICAR Interaction

To test the enrichment for HiCAR identified interactions in significant eQTL-TSS association, the eQTL-TSS associations in H1 hESC were first obtained from DeBoever. C. et al. (2017) Cell Stem Cell. 20:533-546e7. To assess the significance of the enrichment, a null distribution was generated by creating a simulated-interaction datasets by resampling the same number of interactions at random from distance-matched interactions (with 10,000 repeats). The empirical P-value was computed by comparing the observed overlapping number with the null distribution.

15. Machine Learning Approaches to Identity Features Associated with Interaction Activity

Epigenetic features were collected from the public ENCODE consortium from H1 hESC lines. There were 75 ChIP-seq datasets collected for the H1 cell line, including 26 histone mark datasets and 49 transcription factors (redundant datasets from different labs were removed). Average bigWig signals on each 5 KB anchor were computed using the bigWigAverageOverBed command from UCSC. Regression-based machine learning was used. For regression, a sigmoid function was used to scale the chromatin interaction score into a [0,1] range:

$$f ( x ) = \frac{1}{1 + {e}^{- c 1 ( x - c 2 )}}$$

Here, c1=0.05 and c2=20 empirically, such that the bins with stronger interactions had a value closer to 1 after sigmoid conversion. Regression methods were used in the scikit-learn Python package (Pedregosa. F. et al. (2011) J. Machine Learning Res. 12:2825-2830) for regression analysis, including linear regression, decision tree. xbgboost, random forest and linear-kernel support vector machine (SVM). The XGBoost Python package (Chen T, et al. (2016) arXiv [cs.LG]) was used for XGBoost regression analysis. Clusterprofile (Fornes O, et al. (2020) Nucleic Acids Res. 48:D87-D92). was used to examine whether particular gene sets were enriched in certain gene lists. GO categories with “BH” adjusted p-value <0.05 were considered as significant.

### 16. Data Process Pipeline for HiCAR Data

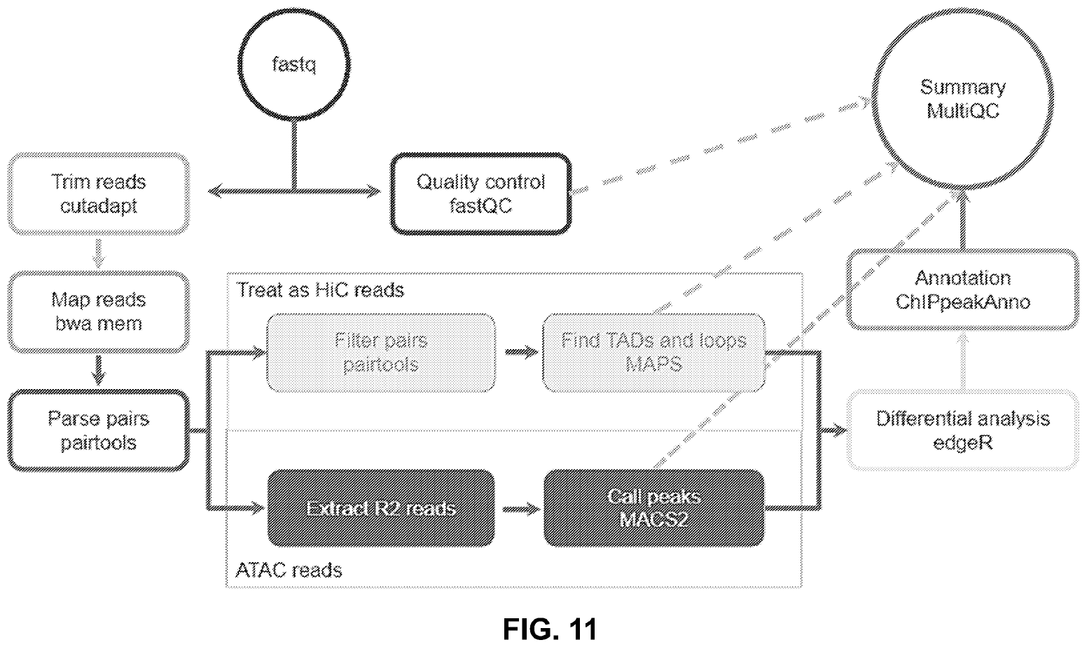

For processing HiCAR data, provided herein is a user-friendly data processing pipeline called HiCARTools (https://github.com/nf-core/hicar). (FIG. 11). HiCARTools or NF-Core/HiCAR is a bioinformatics best-practice analysis pipeline for processing HiCAR data, which is a robust and sensitive multiomic co-assay for the simultaneous analysis of the transcriptome and chromatin accessibility and cis-regulatory chromatin contacts. This pipeline was constructed using Nextflow, which is a workflow tool to run tasks across multiple compute infrastructures in a very portable manner. Nextflow uses Docker/Singularity containers, which made installation trivial and ensured that the results were highly reproducible. The Nextflow DSL2 implementation of this pipeline used one container per process, which made it much easier to maintain and update software dependencies. When possible, these processes were submitted to and installed from nf-core/modules to make them available to all nf-core pipelines and available to everyone within the Nextflow community. On release, automated continuous integration tests run the pipeline on a full-sized dataset on the AWS cloud infrastructure. This ensured that the pipeline ran on AWS, had sensible resource allocation defaults set to run on real-world datasets, and permitted the persistent storage of results to benchmark between pipeline releases and other analysis sources. The results obtained from the full-sized test can then be viewed on nf-core website.

As outlined in FIG. 11, the analysis pathway generally comprises the following steps: (1) Read QC (FastQC); (2) Trim reads (cutadapt); (3) Map reads (bwa mem); (4) Filter reads (pairtools); (5) Quality analysis (pairsqc); (6) Create cooler files for visualization (cooler); (7) Call peaks for ATAC reads (R2 reads) (MACS2); (8) Find TADs and loops (MAPS): (9) Differential analysis (edgeR); (10) Present QC for raw reads (MultiQC). The analysis pathway can also comprise annotation of TADs and loops (ChIPpeakAnno). The nf-core framework for community-curated bioinformatics pipelines was previously (Ewels P A, et al. (2020) Nat. Biotech. 38:276-278).

### Example 1

### The Principle and Experimental Design Driving HiCAR

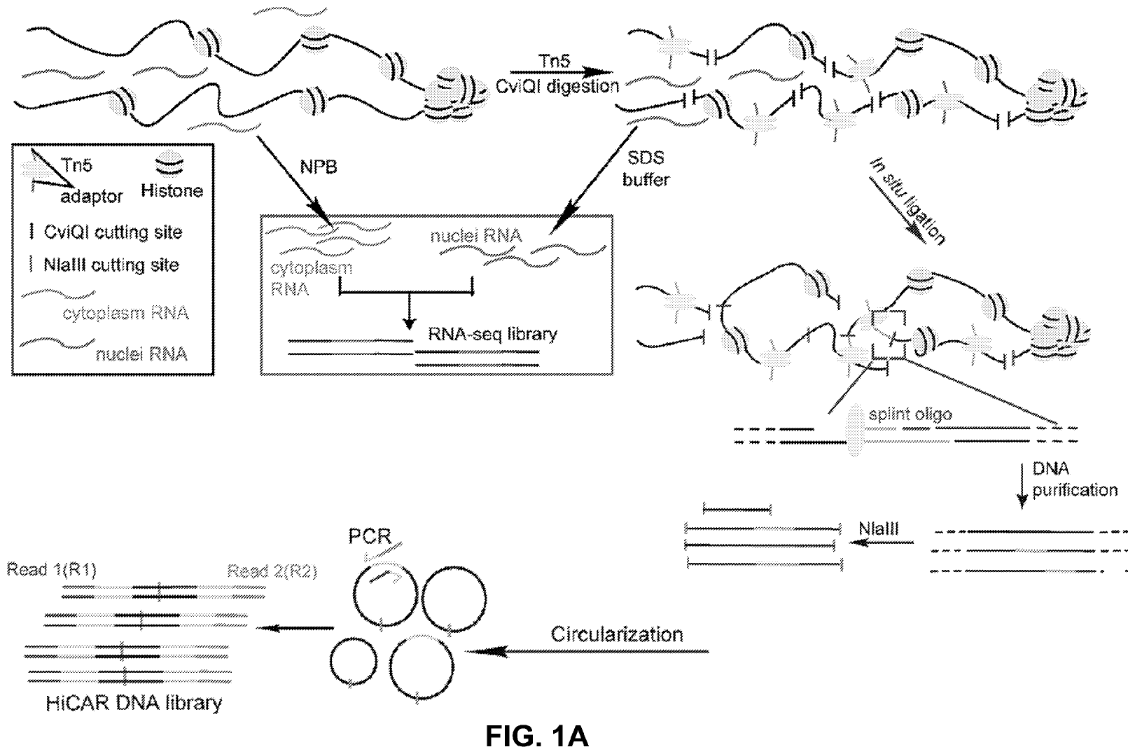

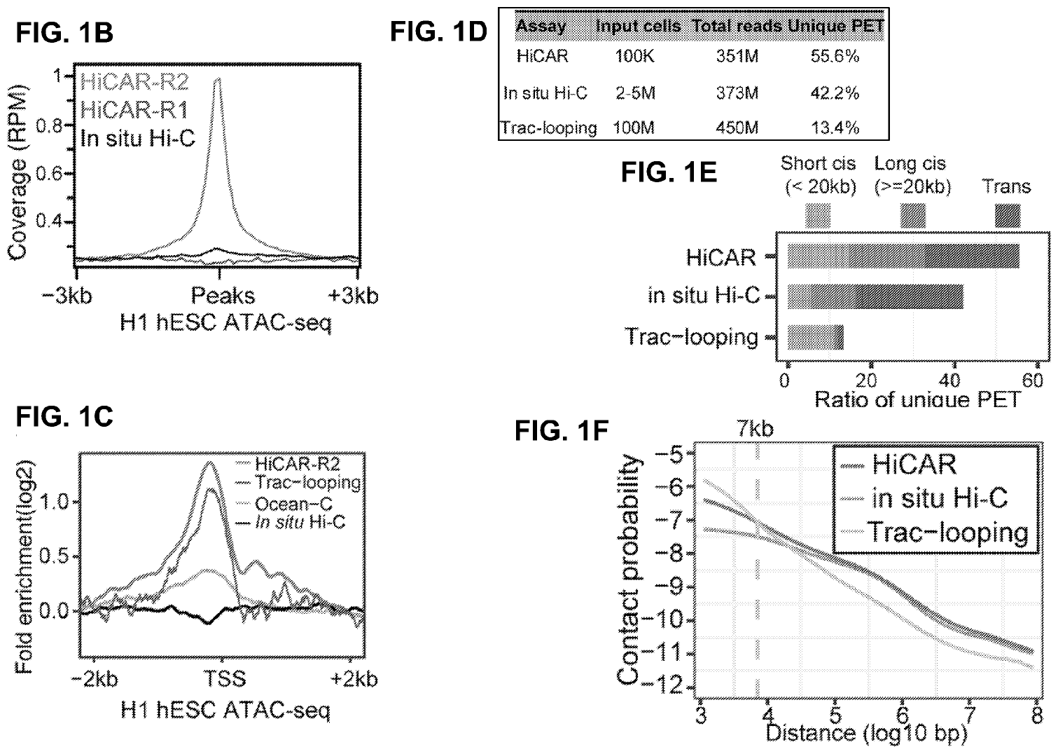

As a proof-of-principle, HiCAR was performed on H1 hESCs, because of the rich public genomic datasets available for this cell line that could be used to benchmark our approach (Table 1), list of public datasets used in this study) (Roadmap Epigenomics Consortium et al. (2015) Nature 518:317-330; ENCODE Project Consortium. (2012) Nature. 489:57-74). First, ˜100,000 cross-linked H1 cells were treated with Tn5 transposase assembled with an engineered DNA adaptor (Table 2). The Tn5 adaptors contained a Mosaic End (ME) sequence for Tn5 recognition (Reznikoff W S. (2003) Mol. Microbiol. 47:1199-1206) as well as a single-stranded flanking sequence that can be ligated to the CviQI-digested DNA fragment with a splint oligo (FIG. 1A, Table 2). Next, restriction enzyme digestion was performed using the 4-base cutter CviQI, followed by in situ proximity ligation to ligate Tn5 adaptor to the proximal genomic DNA. After in situ ligation, crosslinks were reversed and the DNA was purified, digested by another 4-base cutter NIaIII, and circularized by re-ligation. The circularized DNA was used for PCR amplification to generate HiCAR DNA libraries for Next-Generation-Sequencing (NGS). Forward and reverse PCR primers (Table 2) were then used for library amplification, which anneal to the ME sequence and splint oligo sequence, respectively. Therefore, the resulting amplified chimeric DNA fragment contains one end derived from the CviQI digested genomic DNA (captured by Read 1 of each paired-end sequence. FIG. 1A), and one end derived from the Tn5-tagmented open chromatin sequence (captured by Read 2 of each paired-end sequence, FIG. 1A). Additionally, polyA RNAs from the cytoplasm and nucleoplasm were collected during the procedure (FIG. 11A) and subjected to RNA-Seq library preparation using a protocol modified from SMART-seq2 (Picelli S, et al. (2014) Nat. Protoc. 9:171-181) (detailed supra).

TABLE 2
Oligo and DNA Sequences Used in this Study

| Name | Sequence |
|---|---|
| BfaI-truseqR1-pmeI- | /5Phos/TAAGATCGGAAGAGCGTCGTGTttaaaCGGAGATGTGT |
| nextera7 (adapter) | ATAAGAGACAG (SEQ ID NO: 01) |
| Tn5MErev (adapter) | 5Phos/CTGTCTCTTATACACATCT (SEQ ID NO: 02) |
| TruseqR1(splint oligo) | ACACGACGCTCTTCCGATCT (SEQ ID NO: 03) |
| Nextera-pcr-i7-10-L | CAAGCAGAAGACGGCATACGAGATCAGCCTCGGTCTCGTG |
| | GGCTCGGAGATGTGTATAAGAGACAG (SEQ ID NO: 04) |
| NEB primer i501 | AATGATACGGCGACCACCGAGATCTACACTATAGCCTACA |
| | CTCTTTCCCTACACGACGCTCTTCCGATCT (SEQ ID NO: 05) |
| dT30VN-ME-A | TCGTCGGCAGCGTCAGATGTGTATAAGAGACAGNNNNVTT |
| | TTTTTTTTTTTTTTTTTTTTTTTTTTTTVN (SEQ ID NO: 06) |
| NotI-TSO | /5isodG/GCGGCCGCAAGCAGTGGTATCAACGCAGAGTACAT |
| | rGrGrG (SEQ ID NO: 07) |
| 1S PCR | AAGCAGTGGTATCAACGCAGAGT (SEQ ID NO: 08) |
| Tn5ME-A-aHiC | AGCGTCAGATGTGTATAAGAGACAG (SEQ ID NO: 09) |
| Nextera i7 | CAAGCAGAAGACGGCATACGAGATCTAGTACGGTCTCGTG |
| | GGCTCGG (SEQ ID NO: 10) |
| Nextera i5 | AATGATACGGCGACCACCGAGATCTACACCTCTCTATTCGT |
| | CGGCAGCGTC (SEQ ID NO: 11) |
| sox2-gRNA-#1-1F | CACCGGTGTCGTCTTGTCTTTAGTC (SEQ ID NO: 12) |
| sox2-gRNA-#1-1R | AAACGACTAAAGACAAGACGACACC (SEQ ID NO: 13) |
| sox2-gRNA-#1-2F | CACCGggACCATAGGTTCCTAGAGC (SEQ ID NO: 14) |
| sox2-gRNA-#1-2R | AAACGCTCTAGGAACCTATGGTccC (SEQ ID NO: 15) |
| sox2-gRNA-#1-3F | CACCGGGGCAGCCTTGATGTCCTAA (SEQ ID NO: 16) |
| sox2-gRNA-#1-3R | AAACTTAGGACATCAAGGCTGCCCC (SEQ ID NO: 17) |
| sox2-gRNA-#2-1F | CACCGTCCCCGTGCATTGAAGAAAG (SEQ ID NO: 18) |
| sox2-gRNA-#2-1R | AAACCTTTCTTCAATGCACGGGGAC (SEQ ID NO: 19) |
| sox2-gRNA-#2-2F | CACCGCAGGTGTCTTGCCTGCCCTA (SEQ ID NO: 20) |
| sox2-gRNA-#2-2R | AAACTAGGGCAGGCAAGACACCTGC (SEQ ID NO: 21) |
| sox2-gRNA-#2-3F | CACCGGCAGCAGAAGGTTCTTTAGC (SEQ ID NO: 22) |
| sox2-gRNA-#2-3R | AAACGCTAAAGAACCTTCTGCTGCC (SEQ ID NO: 23) |
| sox2-gRNA-#3-1 F | CACCGAAAGCGAGCGCCCTGATTAA (SEQ ID NO: 24) |
| sox2-gRNA-#3-1 R | AAACTTAATCAGGGCGCTCGCTTTC (SEQ ID NO: 25) |
| sox2-gRNA-#3-2 F | CACCGTCCCGGGAGTAACGAGCAAG (SEQ ID NO: 26) |
| sox2-gRNA-#3-2 R | AAACCTTGCTCGTTACTCCCGGGAC (SEQ ID NO: 27) |
| sox2-gRNA-#3-3 F | CACCGGTTACTCCCGGGAGAGGCGC (SEQ ID NO: 28) |
| sox2-gRNA-#3-3 R | AAACGCGCCTCTCCCGGGAGTAACC (SEQ ID NO: 29) |

HiCAR libraries were made from 3 biological replicates of H1 hESC and each library was sequenced to a depth of ˜300 million pair-end raw reads (Table 3). The enrichment of HiCAR reads around open chromatin regions defined by H1 ESC ATAC-se data generated by the 4DN consortium (Krietenstein N, et al. (2020) Mol. Cell. 78:554-565.e7) was first examined.

TABLE 3
Summary of Seven HiCAR DNA Libraries Generated
with H1 hESC, GM12878, and mESCs

| | | Uniquely | | | |
|---|---|---|---|---|---|
| | | Mapped | | | |
| | Total | & Non- | | | cis |
| Sample | Reads | Redundant | Trans | Cis | >20 KB |
| H1_ | 351,774,247 | 195,488,040 | 79,630,309 | 115,857,731 | 64,262,169 |
| HiCAR_rep1 | | | | | |
| H1_ | 319,485,025 | 193,662,148 | 81,078,965 | 112,583,183 | 56,349,290 |
| HiCAR_rep2 | | | | | |
| H1_ | 251,385,290 | 121,567,605 | 48,170,227 | 73,397,378 | 39,388,900 |
| HiCAR_rep3 | | | | | |
| GM12878_ | 295,942,008 | 114,029,536 | 44,071,441 | 69,958,095 | 38,992,777 |
| HiCAR_rep1 | | | | | |
| GM12878_ | 306,222,253 | 124,968,330 | 44,695,381 | 80,272,949 | 45,739,034 |
| HiCAR_rep2 | | | | | |
| mESC | 371,410,011 | 132,435,481 | 25,309,335 | 107,126,146 | 61,326,344 |
| HiCAR_rep1 | | | | | |
| mESC | 430,477,951 | 154,726,871 | 29,119,298 | 125,607,573 | 71,519,222 |
| HiCAR_rep2 | | | | | |

Read 1 (R1) and Read 2 (2) of the HiCAR DNA library were separately analyzed and the publicly available H1 hESC insitu Hi-C data from the 4DN consortium (Krietenstein N, et al. (2020) Mol. Cell. 78:554-565.e7) (Table 1) was used as a reference dataset without targeted enrichment.

TABLE 1
The List of Public Datasets Used in this Study

| Cell Lines | Assay | Target | Resource | Reference |
|---|---|---|---|---|
| H1 | ATAC-seq | open chromatin regions | 4dnucleome | 4DNESLMCRW2C |
| H1 | in situ HiC | chromatin interactions | 4dnucleome | 4DNES2M5JIGV |
| H1 | RNA-Seq | RNA profile | encode | ENCSR000BZU |
| H1 | DNase Hi-C | chromatin interactions | GEO | GSE56869 |
| H9 | HiChIP | H3K4me1 | GEO | GSE105028 |
| H9 | HiChIP | CTCF | GEO | GSE105028 |
| T cells | trac-looping | chromatin interactions | GEO | GSE87254 |
| GM12878 | OCEAN-C | chromatin interactions | GEO | GSE100832 |
| GM12878 | in situ HiC | in situ HiC | GEO | GSB63525 |
| GM12878 | ATAC-seq | open chromatin regions | GEO | GSB47753 |
| GM12878 | HiChIP | Smc1a | GEO | GSE80820 |
| mESC | PLAC-seq | CTCF | GEO | GSB119663 |
| mESC | PLAC-seq | H3K4me3 | GEO | GSB119663 |
| mESC | in situ HiC | chromatin interactions | 4dnucleome | 4DNESDXUWBD9 |
| mESC | ATAC-seq | open chromatin regions | GEO | GSE66581 |
| H1 | chip-seq | ATF3-human | ENCODE | ENCFF481EHX |
| H1 | chip-seq | BACH1-human | ENCODE | ENCFF594ALF |
| H1 | chip-seq | BRCA1-human | ENCODE | ENCFF620MRE |
| H1 | chip-seq | CHD1-human | ENCODE | ENCFF563QHP |
| H1 | chip-seq | CHD2-human | ENCODE | ENCFF318NSO |
| H1 | chip-seq | CHD7-human | ENCODE | ENCFF575OWE |
| H1 | chip-seq | CTBP2-human | ENCODE | ENCFF562PRB |
| H1 | chip-seq | CTCF-human | ENCODE | ENCFF473IZV |
| H1 | chip-seq | EGR1-human | ENCODE | ENCFF341OGJ |
| H1 | chip-seq | EP300-human | ENCODE | ENCFF491ZOF |
| H1 | chip-seq | FOSL1-human | ENCODE | ENCFF498IQF |
| H1 | chip-seq | GABPA-human | ENCODE | ENCFF401DOJ |
| H1 | chip-seq | GTF2F1-human | ENCODE | ENCFF173BEC |
| H1 | chip-seq | HDAC2-human | ENCODE | ENCFF948IYF |
| H1 | chip-seq | JUN-human | ENCODE | ENCFF815WEI |
| H1 | chip-seq | JUND-human | ENCODE | ENCFF128BVN |
| H1 | chip-seq | KDM1A-human | ENCODE | ENCFF222RPJ |
| H1 | chip-seq | KDM5A-human | ENCODE | ENCFF825WLX |
| H1 | chip-seq | MAFK-human | ENCODE | ENCFF640RNH |
| H1 | chip-seq | MAX-human | ENCODE | ENCFF444FFZ |
| H1 | chip-seq | MYC-human | ENCODE | ENCFF878ZL [symbol] |
| H1 | chip-seq | NANOG-human | ENCODE | ENCFF305LHR |
| H1 | chip-seq | NRF1-human | ENCODE | ENCFF51 [symbol] ERL |
| H1 | chip-seq | PHF8-human | ENCODE | ENCFF935JRI |
| H1 | chip-seq | POLR2A-human | ENCODE | ENCFF379IRQ |
| H1 | chip-seq | POLR2AphosphoS5- | ENCODE | ENCFF655OPV |
| | | human | | |
| H1 | chip-seq | RAD21-human | ENCODE | ENCFF913JGA |
| H1 | chip-seq | RBBP5-human | ENCODE | ENCFF076ZMU |
| H1 | chip-seq | REST-human | ENCODE | ENCFF600PQH |
| H1 | chip-seq | RFX5-human | ENCODE | ENCFF027CMH |
| H1 | chip-seq | RNF2-human | ENCODE | ENCFF308TCO |
| H1 | chip-seq | RXRA-human | ENCODE | ENCFF134SMY |
| H1 | chip-seq | SAP30-human | ENCODE | ENCFF779YFX |
| H1 | chip-seq | SIN3A-human | ENCODE | ENCFF350OAA |
| H1 | chip-seq | SIX5-human | ENCODE | ENCFF665USC |
| H1 | chip-seq | SP1-human | ENCODE | ENCFF256MVQ |
| H1 | chip-seq | SRF-human | ENCODE | ENCFF941KEV |
| H1 | chip-seq | SUZ12-human | ENCODE | ENCFF723MAM |
| H1 | chip-seq | TAF1-human | ENCODE | ENCFF689QWC |
| H1 | chip-seq | TAF7-human | ENCODE | ENCFF160JKQ |
| H1 | chip-seq | TBP-human | ENCODE | ENCFF052TRV |
| H1 | chip-seq | TCF12-human | ENCODE | ENCFF715MYQ |
| H1 | chip-seq | USF1-haman | ENCODE | ENCFF133IZI |
| H1 | chip-seq | USF2-human | ENCODE | ENCFF757FPX |
| H1 | chip-seq | YY1-human | ENCODE | ENCFF406PYH |
| H1 | chip-seq | ZNF143-human | ENCODE | ENCFF377SDG |
| H1 | chip-seq | ZNF274-human | ENCODE | ENCFF040IXF |
| H1 | chip-seq | H2AK5ac-human | ENCODE | ENCFF508WLD |
| H1 | chip-seq | H2BK120ac-human | ENCODE | ENCFF757EYT |
| H1 | chip-seq | H2BK12ac-human | ENCODE | ENCFF873OYG |
| H1 | chip-seq | H2BK15ac-human | ENCODE | ENCFF236YZE |
| H1 | chip-seq | H2BK20ac-human | ENCODE | ENCFF382G [symbol] P |
| H1 | chip-seq | H2BK5ac-human | ENCODE | ENCFF451CYN |
| H1 | chip-seq | H3K14ac-human | ENCODE | ENCFF605ROH |
| H1 | chip-seq | H3K18ac-human | ENCODE | ENCFF413LVW |
| H1 | chip-seq | H3K23ac-human | ENCODE | ENCFF464QEO |
| H1 | chip-seq | H3K23me2-human | ENCODE | ENCFF517UOA |
| H1 | chip-seq | H3K27ac-human | ENCODE | ENCFF986PCY |
| H1 | chip-seq | H3K27me3-human | ENCODE | ENCFF502GXT |
| H1 | chip-seq | H3K36me3-human | ENCODE | ENCFF141YAA |
| H1 | chip-seq | H3K4ac-human | ENCODE | ENCFF571UTM |
| H1 | chip-seq | H3K4me1-buman | ENCODE | ENCFF593OAZ |
| H1 | chip-seq | H3K4me2-human | ENCODE | ENCFF502TJG |
| H1 | chip-seq | H3K4me3-human | ENCODE | ENCFF623ZAW |
| H1 | chip-seq | H3K56ac-human | ENCODE | ENCFF688YVV |
| H1 | chip-seq | H3K79me1-human | ENCODE | ENCFF349YSW |
| H1 | chip-seq | H3K79me2-human | ENCODE | ENCFF833AVU |
| H1 | chip-seq | H3K9ac-human | ENCODE | ENCFF834AZA |
| H1 | chip-seq | H3K9me3-human | ENCODE | ENCFF435YZW |
| H1 | chip-seq | H4K20me1-human | ENCODE | ENCFF772CZB |
| H1 | chip-seq | H4K5ac-human | ENCODE | ENCFF114DFQ |
| H1 | chip-seq | H4K8ac-human | ENCODE | ENCFF510WQU |
| H1 | chip-seq | H4K91ac-human | ENCODE | ENCFF068LXN |
| H1 | chip-seq | OCT4-human | cistrome | CistromeDB: 4924 |
| H1 | chip-seq | SOX2-human | cistrome | CistromeDB: 4931 |

[symbol]

indicates data missing or illegible when filed

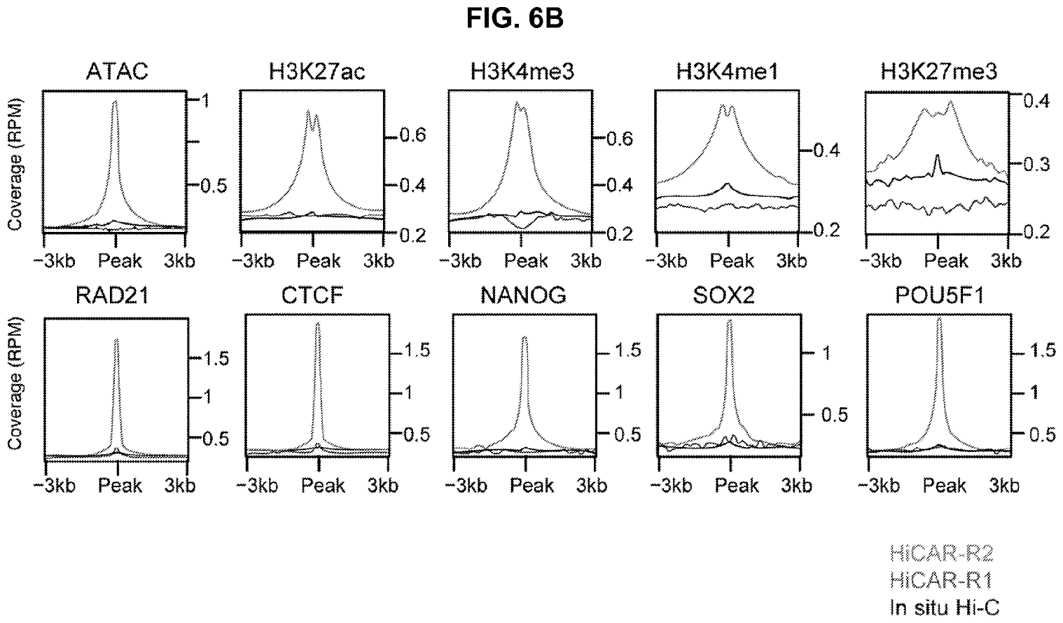

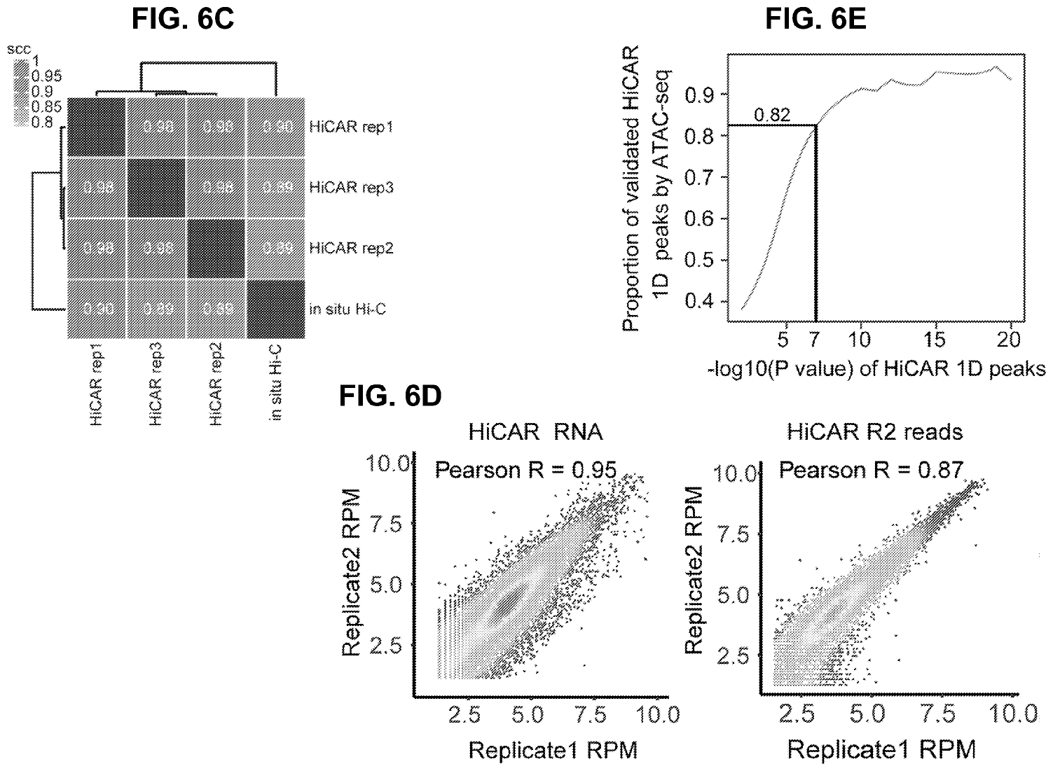

As expected, HiCAR R2 reads were highly enriched at the H1 hESC ATAC-seq peaks (FIG. 1B), while the, R1 reads and in situ Hi-C reads show no enrichment (FIG. 11B). This result confirmed that HiCAR successfully captured and enriched the interactions between open chromatin regions (R2) and other genomic regions (R1). The interactions described below are referred to as “open-to-all” interactions. This was different from Trac-looping (Lai B, et al. (2018) Nat. Methods. 15:741-747), a different method capturing “open-to-open” interactions between pairs of open chromatin regions. The enrichment efficiency of HiCAR was then compared to that of Trac-looping and Ocean-C, two methods recently developed for mapping long-range interactions anchored at open chromatin regions (Lai B, et al. 2018; Li T, et al. (2018) Genome Biol. 19:54). Because HiCAR, Trac-looping, and Ocean-C experiments were performed in different cell lines, the open chromatin enrichment efficiency of each method was assessed by examining transcription start site (TSS) signal enrichment. TSS signal enrichment is a metric widely used as a quality control standard to compare signal-to-noise ratios of ATAC-seq data across different cell types (Corces M R, et al. (2017) Nat. Methods. 14:959-962). Both HiCAR and Trac-looping reads showed high TSS signal enrichment (FIG. 1C, log 2 fold change 1.02 and 0.84. respectively, Wilcoxon test, both p<2.2e-16), while Ocean-C reads showed significant but much weaker enriched signal on TSS (FIG. 1C. log 2 fold change=0.30, Wilcoxon test p<2.2e-16). A similar analysis was then conducted by comparing HiCAR data to the public DNase Hi-C data (FIG. 6A). DNase Hi-C was previously determined not to introduce open chromatin bias into the chromatin contact matrix (Ma W, et al. (2015) Nat. Methods. 12:71-78). Consistent with these results, the DNase Hi-C reads were indeed not enriched on TSS regions (FIG. 6A. brown line).

A similar analysis to compare HiCAR data to the public HiChIP and PLAC-seq data (FIG. 6A) was also performed. As expected, the signal enrichment of HiChIP and PLAC-seq at cis-regulatory sequences depended on the antibody used for chromatin immunoprecipitation (ChIP). For example, H3K4me3 modification is the mark of promoters (Heintzman N D, et al. (2007) Nat. Genet. 39:311-318), and the sequencing reads from H3K4me3 PLAC-seq data exhibited significant enrichment around TSS regions (FIG. 6A, black line). whereas H3K4mel (enhancer mark) HiChiP reads showed no enrichment on TSS (FIG. 6A, purple line). Since open chromatin regions are bound by multiple TF and histone marks (Klemm S L, et al. (2019) Nat. Rev. Genet. 20:207-220). HiCAR reads were expected to enrich comprehensive epigenome signatures associated with cis-regulatory sequences. Accordingly, HiCAR R2 reads, but not R1 reads, were highly enriched on H1 hESC H3K27ac, H3K3mel, H3K4me3, H3K27me3, RAD21, CTCF. NANOG, SOX2, and POU5F1 ChIP-seq peaks (FIG. 63). These results demonstrated that while HiChIP and PLAC-seq only enriched the reads that were bound by the specific ChIP antibody. HiCAR effectively enriched a broader array of reads anchored at open chromatin regions (FIG. 1C) and associated with a spectrum of epigenetic modifications and transcription factor binding (FIG. 6A).

Given the relatively low TSS-enrichment efficiency of Ocean-C (FIG. 1C), Ocean-C was excluded from the following analysis. Only HiCAR data was compared to the public Trac-looping data (Lai B, et al. 2018). One in situ Hi-C library (that was generated by the 4DN consortium (Dekker J, et al. (2017) Nature. 549:219-226) and sequenced at similar depth (FIG. 1D, 373 million raw reads)) was included as control data without targeted enrichment. Notably, HiCAR required much less input material (100 thousand cells) than Trac-looping (100 million cells) and in situ Hi-C (2-5 million cells), while producing 4.15-fold more uniquely mapped PETs than Trac-looping (FIG. 1D. 55.6% versus 13.4%). More importantly, compared to Trac-looping, HiCAR captured about 17-fold (18.3% versus 1.1%, blue bars in FIG. 1E) more long-range (>20 KB) cis-PET, which are the informative reads to identify long-range chromatin interactions. Furthermore, the genome-wide average contact frequency captured by HiCAR, in situ Hi-C, and Trac-looping was examined. HiCAR and in situ Hi-C showed similar decay rate in capturing long-range chromatin interactions with increased linear genomic distance (FIG. 1F), while Trac-looping captured more short-rage (less than 7 KB) chromatin contacts but fewer long-range interactions (FIG. 1F). Overall, HiCAR outperformed Trac-looping and allowed for efficient and comprehensive capture of cis-regulatory chromatin contacts independent of antibody immunoprecipitation using low-input cells.

### Example 2

### HICAR Faithfully Recapitulated the Key Features of High-Order Chromatin Organization

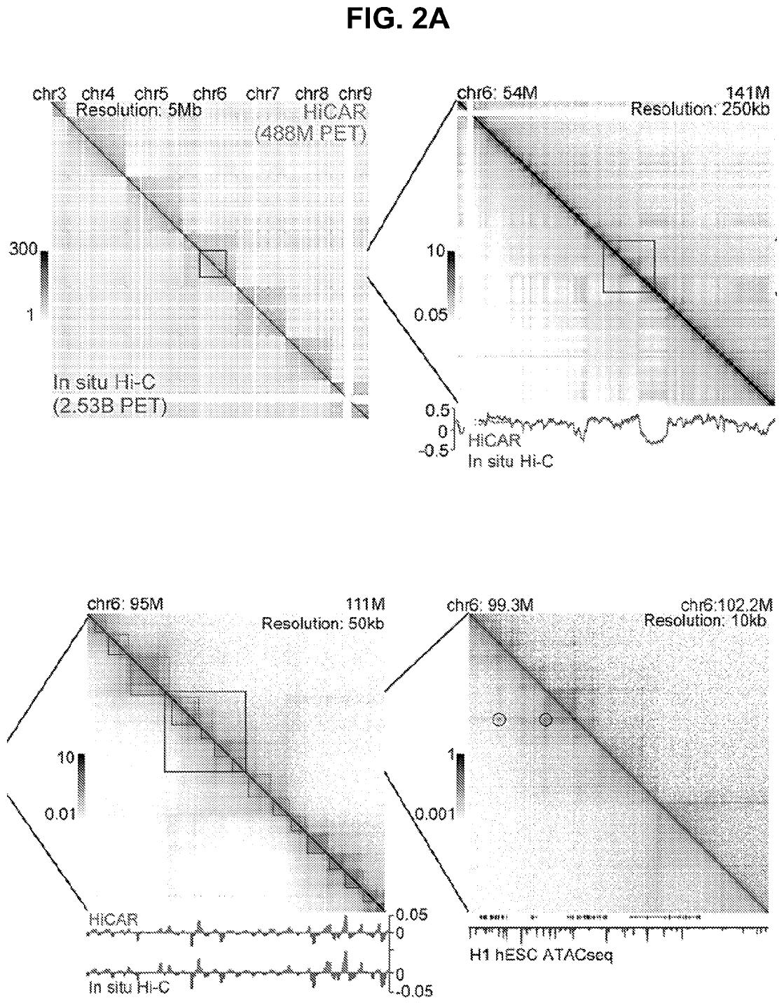

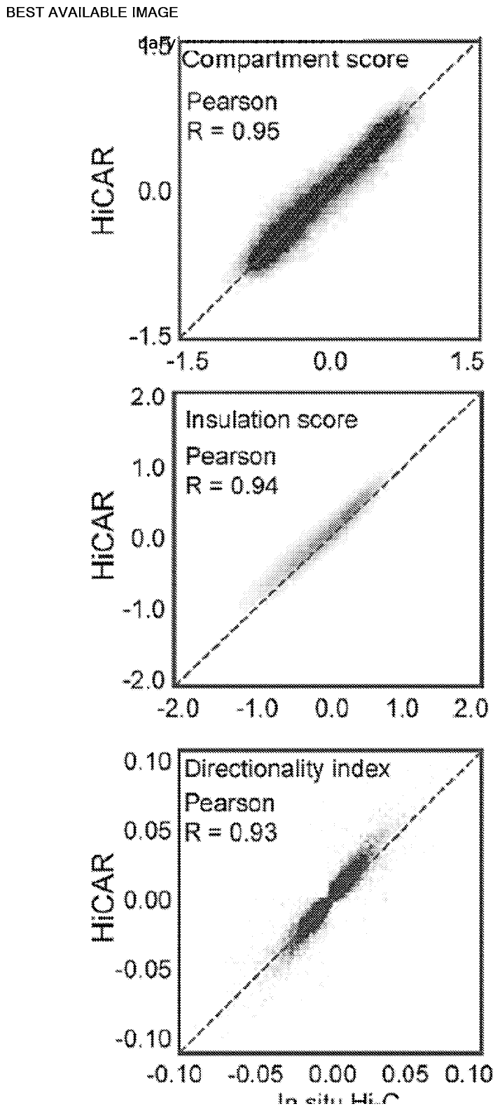

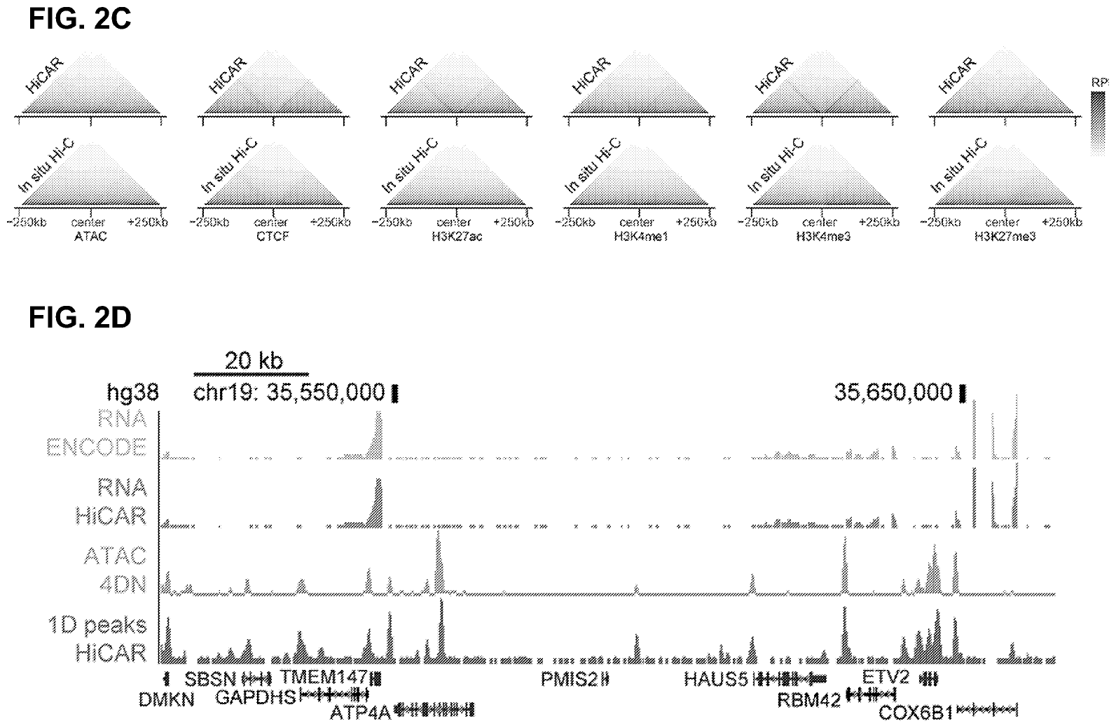

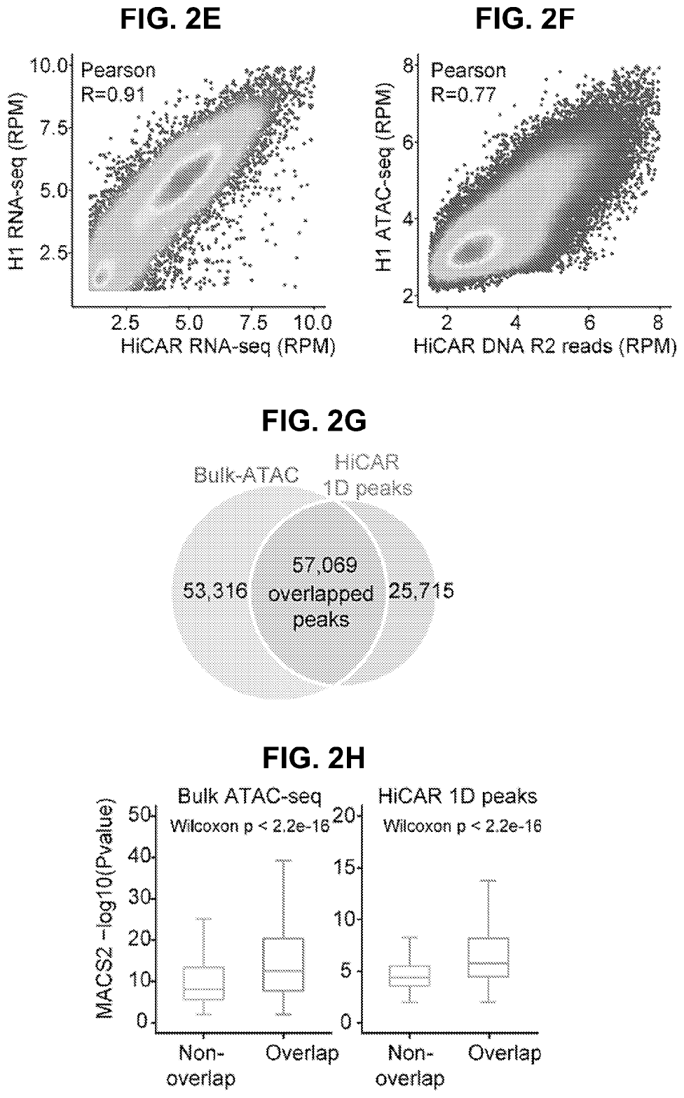

Whether HiCAR could identify the key features of genome architecture was examined. To probe this question, the deeply sequenced (total of 6.2 billion raw reads, generated by 4DN consortium 20) in situ Hi-C data generated from H1 hESCs was used as a “gold standard” in the analysis. The global chromatin contact matrix (sequencing depth normalized) of HiCAR and in situ Hi-C was first visually examined (FIG. 2A). HiCAR generated a chromatin contact matrix highly similar to that of in situ Hi-C at chromosomes, compartments, topological associated domains (TADs), and 10 KB-bin resolutions (FIG. 2A, left to right). To further quantify the similarity of the HiCAR and Hi-C contact matrices, HiCRep (Yang T, et al. (2017) Genome Res. 27:1939-1949) was used to compute the stratum-adjusted correlation coefficient (SCC) among three HiCAR replicates and the in situ Hi-C data (Krietenstein N, et al. 2020). At the genome-wide scale, the three biological replicates of HiCAR library were highly reproducible (FIG. 6C, SCC=0.98), and HiCAR captured a chromatin interaction pattern similar to the deeply sequenced in situ Hi-C dataset (FIG. 6C, SCC=0.90, 0.89, 0.89). Further analysis revealed that the A/B compartment PC1 score, insulation score, and directionality index calculated from the HiCAR and in situ Hi-C data were well correlated with each other (FIG. 2B).

Notably, the HiCAR contact matrix, built from 488 million uniquely mapped PETs, revealed as much, if not greater, details on chromatin interactions compared to the deeply sequenced (2.53 billion uniquely mapped PETs) in situ Hi-C data (FIG. 2A). Whether HiCAR could enrich the long range cis-PETs anchored on cREs was then evaluated To probe this question, the open chromatin peaks and ChIP-seq peaks of 1l hESC was identified by ATAC-seq and ChIP-seq datasets (including CTCF, H3K27ac, H3K4me1, H3K4me3, and H3K27me3 ChIP-seq), and set these peaks as the center of the sub-chromatin contact matrix expanding +/−250 KB window from each peak center. Next, the PET signal (sequencing depth normalized) from all the sub-chromatin contact matrices was aggregated. Interestingly, the aggregated HiCAR PET signal showed a clear stripe pattern extending from the peak centers of all the examined epigenetic features (FIG. 2C, top tracks). By contrast, the stripe patterns of PET signal from the aggregated Hi-C contact matrices were much weaker (FIG. 2C, bottom track). Compared to in situ Hi-C, HICAR effectively enriched long-range cis-PETs anchored at cis-regulatory sequences and associated with diverse histone modification and TF binding.

### Example 3

### HICAR Yielded Both High-Quality Chromatin Accessibility and Transcriptome Data from the Same Input Biological Sample

In the HiCAR DNA library, the R2 reads were derived from the genomic sequences targeted by Tn5 tagmentation (FIG. 1A). Therefore, the R2 reads could be treated as the single-end ATAC-seq reads to map genome-wide open chromatin regions. In a HiCAR experiment, the cytoplasm and nucleoplasm ployA-RNA could be collected for RNA-Seq library preparation (FIG. 1A, detailed in material and methods). After deep sequencing, the HiCAR RNA-Seq data and the DNA R2 reads were confirmed to be highly reproducible between biological replicates (FIG. 6D, Pearson correlation coefficient=0.95 for RNA and 0.87 for R2 reads). Next, the HiCAR RNA-Seq data were compared to the public H1 hESC RNA-Seq data (by ENCODE), and the DNA library R2 reads were compared to the ATAC-seq data (by the 4DN consortium). As shown in FIG. 2D. very similar patterns of RNA and open chromatin signals on genome browser were observed. At the genome-wide scale, the HiCAR RNA-Seq data and the DNA R2 reads were highly correlated with the bulk RNA-Seq and ATAC-seq datasets (FIG. 2E—PCC=0.91, FIG. 2F—PCC=0.77). Then, MACS2 (Zhang Y, et al. (2008) Genome Biol. 9:R137) was used to call ID open chromatin peaks from HICAR R2 reads and compared to the ATAC-seq peaks. As shown in FIG. 2G, 57,069 (68.9% of total) HiCAR ID peaks overlapped with ATAC-seq peaks. Further analysis revealed that the overlapping peaks were associated with more significant p-values (MACS2) in both ATAC-seq and HiCAR 1 D peaks (FIG. 2H). When the HiCAR ID peaks were ranked based on their MACS2 p-value, more than 82% of the high confidence ID peaks (p-value <10e-7) were validated by ATAC-seq peaks (FIG. 6E). Taken together, HiCAR generated high-quality chromatin accessibility and transcriptome data using a singular low-input sample. This is a technical advancement over the state of the art.

### Example 4

### Identification of Long-Range Cis-Regulatory Chromatin Interactions in H1 hESC Using HICAR

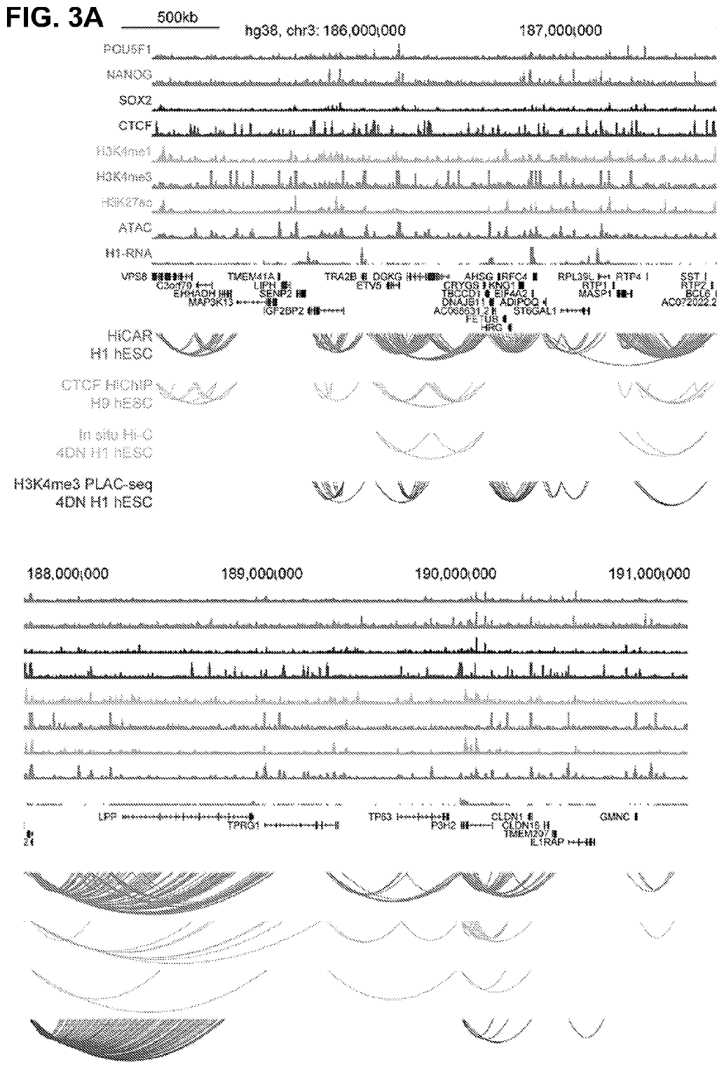

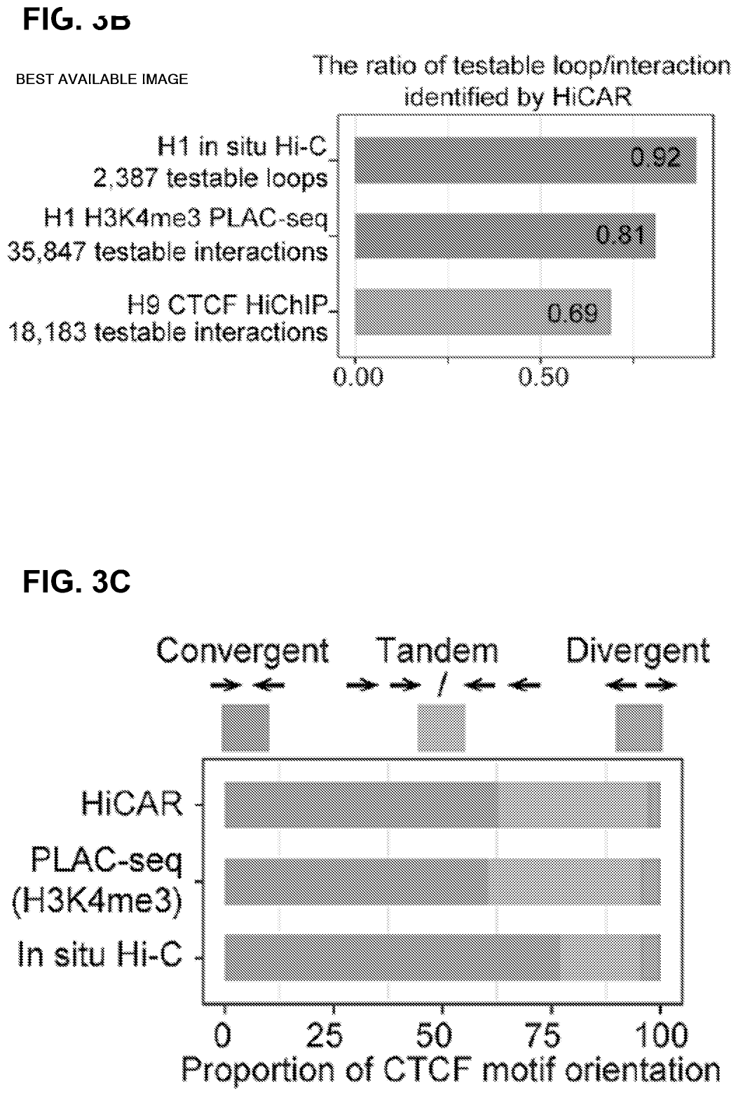

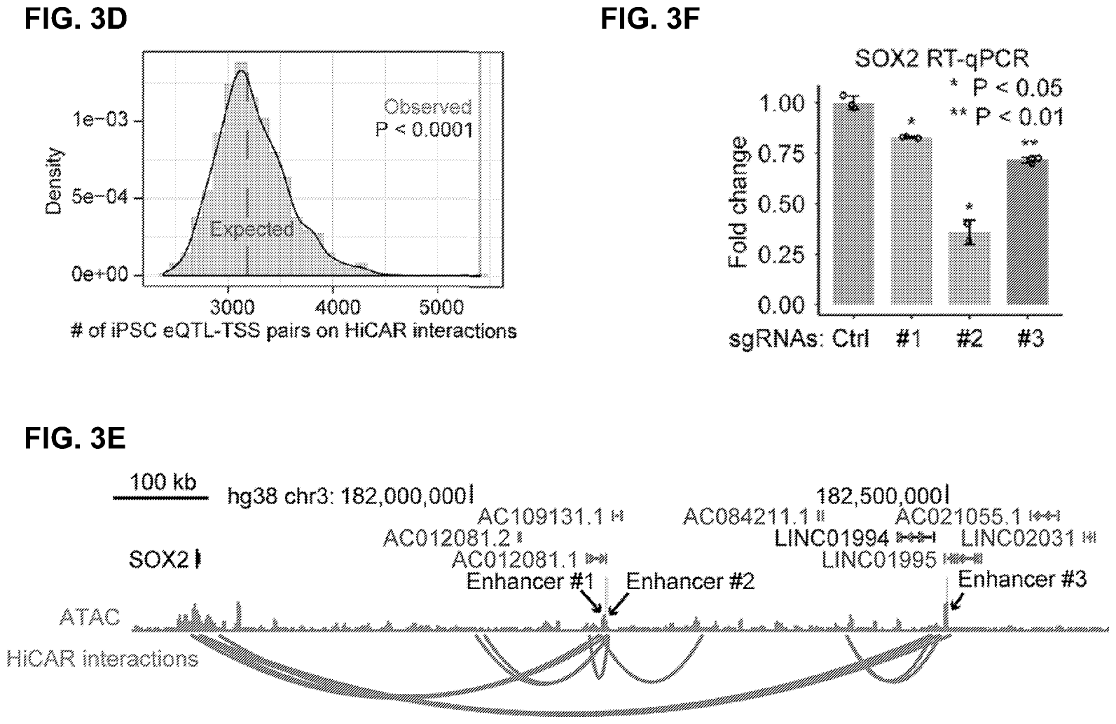

HiCAR is designed to identify the long-range chromatin interactions anchored at cREs at high-resolution. To achieve this goal, MAPS, a method recently developed for HiChIP and PLAC-seq data, was applied to the HiCAR dataset. Using MAPS, the potential systemic biases were first removed from the contact matrix, including GC content, sequence mappability, ID chromatin accessibility, and the density of restriction enzyme cutting (detailed in material and methods). In total, 46,792 significant (MAPS FDR <0.01) chromatin interactions were identified at 5 KB resolution and anchored on H1 hESC open chromatin regions (Table 4A). Next, the sensitivity of HiCAR in detecting known chromatin interactions was evaluated. Since there was no “gold standard” set of true positive interactions, HiCAR interactions were compared to chromatin interactions defined by well-established methods such as in situ Hi-C, PLAC-seq, and HiChIP in matched cell types. Specifically, the public in situ Hi-C and H3K4m3 PLAC-seq data generated from H1 hESC by the 4DN consortium was used as was the previously generated CTCF HiChIP data from H9 hESC (Krietenstein et al. (2020); Lyu X, et al. (2018) Mol. Cell. 71:940-955.e7). Due to the lower sequencing depth of some public datasets, the chromatin interactions at 10 KB (Table 48) rather than 5 KB (Table 4A) resolution was employed. In situ Hi-C data (Table 4D) was processed by HiCCUPS while HiChIP (Table 4C) and PLAC-seq data (Table 4E) was processed by MAPS. By visual examination of HiCCUPS loops and MAPS interactions in genome browser, HiCAR interactions showed a similar pattern of loops and interactions identified by these well-established and widely used methods (FIG. 3A. Interestingly, HiCCUPS loops (from in situ Hi-C data) and MAPS interactions (from H3K4me3 PLAC-seq and CTCF HiChiP data) represented a subset of the significant interactions identified by HiCAR (FIG. 3A). To further quantify the sensitivity of HiCAR interactions, the in situ Hi-C loops and HiChIP/PLAC-seq interactions as filtered and only the “testable” loops and interactions with at least one anchor overlapping with ATAC-seq peaks were kept for the following analysis. HiCAR identified 92%, 81%, and 69% of the “testable” loops and interactions identified by in situ Hi-C, H3K4me3 PLAC-seq, and CTCF HiChIP data, respectively (FIG. 38). These results indicated that HiCAR was a highly sensitive method in detecting “known” chromatin interactions identified by well-established methods. Each of Tables 4A-4D are representative of the data generated in the analysis. Each of Tables 4A-4D represents a “snapshot” of the expansive volume of data generated during an analysis. As disclosed supra, HiCARTools or NF-Core/HiCAR is a bioinformatics best-practice analysis pipeline for processing these data.

TABLE 4A
Representative List of Chromatin Loops and Interactions in H1 hESCs Identified in HiCAR Data (5 KB)

| | | | | | | | | | | | | | Clus- |
|---|---|---|---|---|---|---|---|---|---|---|---|---|---|
| | | | | | | | | | | Clus- | | | ter |
| | | | | | | | | | Cluster | ter | Cluster | ClusterNeg | Sum |
| chr1 | start1 | end1 | chr2 | start2 | end2 | count | expected | fdr | Label | Size | Type | Log10P | mit |
| chr1 | 14765000 | 14769999 | chr1 | 15025000 | 15029999 | 14 | 2.10463183 | 1.16E−05 | chr1_01 | 1 | Singleton | 8.06575201 | 1 |
| chr1 | 24070000 | 24074999 | chr1 | 24390000 | 24394999 | 18 | 2.85612445 | 5.36E−07 | chr1_02 | 1 | Singleton | 9.57364853 | 1 |
| chr1 | 34785000 | 34789999 | chr1 | 34850000 | 34854999 | 34 | 8.61975413 | 3.44E−08 | chr1_03 | 1 | Singleton | 10.8976001 | 1 |
| chr1 | 48570000 | 48574999 | chr1 | 48915000 | 48919999 | 12 | 1.70195993 | 4.49E−05 | chr1_04 | 1 | Singleton | 7.38791382 | 1 |
| chr1 | 10645000 | 10649999 | chr1 | 10800000 | 10804999 | 19 | 3.28121829 | 7.66E−07 | chr1_05 | 1 | Singleton | 9.40066924 | 1 |
| chr1 | 16615000 | 16619999 | chr1 | 17010000 | 17014999 | 16 | 3.31972958 | 9.09E−05 | chr1_06 | 1 | Singleton | 7.03064122 | 1 |
| chr1 | 18280000 | 18284999 | chr1 | 18290000 | 18294999 | 70 | 35.3472652 | 8.60E−05 | chr1_07 | 1 | Singleton | 7.05942414 | 1 |
| chr1 | 17915000 | 17919999 | chr1 | 18580000 | 18584999 | 13 | 1.55365615 | 2.68E−06 | chr1_08 | 1 | Singleton | 8.78586553 | 1 |
| chr1 | 19390000 | 19394999 | chr1 | 19580000 | 19584999 | 9 | 2.95306005 | 1.53E−07 | chr1_09 | 1 | Singleton | 10.1745998 | 1 |
| chr1 | 20365000 | 20369999 | chr1 | 20430000 | 20434999 | 23 | 6.14419542 | 4.23E−05 | chr1_010 | 1 | Singleton | 7.41618692 | 1 |
| chr1 | 21900000 | 21904999 | chr1 | 21910000 | 21914999 | 100 | 34.3873798 | 2.70E−16 | chr1_011 | 1 | Singleton | 19.554758 | 1 |
| chr1 | 22670000 | 22674999 | chr1 | 22885000 | 22889999 | 17 | 2.61161451 | 8.67E−07 | chr1_012 | 1 | Singleton | 9.33938886 | 1 |
| chr1 | 29245000 | 29249999 | chr1 | 29255000 | 29259999 | 71 | 33.2920991 | 6.19E−06 | chr1_013 | 1 | Singleton | 8.37643347 | 1 |
| chr1 | 29245000 | 29249999 | chr1 | 29280000 | 29284999 | 48 | 18.1913463 | 2.83E−06 | chr1_014 | 1 | Singleton | 8.75733822 | 1 |
| chr1 | 29240000 | 29244999 | chr1 | 29415000 | 29419999 | 19 | 3.34954155 | 1.04E−06 | chr1_015 | 1 | Singleton | 9.2508096 | 1 |
| chr1 | 31395000 | 31399999 | chr1 | 31545000 | 31549999 | 19 | 4.42212212 | 5.45E−05 | chr1_016 | 1 | Singleton | 7.28741073 | 1 |
| chr1 | 31415000 | 31419999 | chr1 | 31545000 | 31549999 | 33 | 4.38953842 | 3.01E−15 | chr1_017 | 1 | Singleton | 18.4708999 | 1 |

TABLE 4B
Representative List of Chromatin Loops and Interactions in H1 hESCs Identified in HiCAR Data (10 KB)

| | | | | | | | | | | | | | Clus- |
|---|---|---|---|---|---|---|---|---|---|---|---|---|---|
| | | | | | | | | | Clus- | Clus- | | | ter |
| | | | | | | | | | ter | ter | Cluster | ClusterNeg | Sum- |
| chr1 | start1 | end1 | chr2 | start2 | end2 | count | expected | fdr | Label | Size | Type | Log10P | mit |
| chr1 | 4030000 | 4039999 | chr1 | 4600000 | 4609999 | 13 | 2.29628916 | 8.1883E−05 | chr1_01 | 1 | Singleton | 6.76592682 | 1 |
| chr1 | 10500000 | 10509999 | chr1 | 11060000 | 11069999 | 15 | 3.04152509 | 7.5479E−05 | chr1_02 | 1 | Singleton | 6.80629975 | 1 |
| chr1 | 16080000 | 16089999 | chr1 | 16110000 | 16119999 | 97 | 51.7011646 | 4.4938E−06 | chr1_03 | 1 | Singleton | 8.18893492 | 1 |
| chr1 | 16530000 | 16539999 | chr1 | 16630000 | 16639999 | 44 | 13.2314819 | 7.9889E−09 | chr1_04 | 1 | Singleton | 11.205935 | 1 |
| chr1 | 18320000 | 18329999 | chr1 | 18480000 | 18489999 | 44 | 11.1113601 | 3.6057E−11 | chr1_05 | 1 | Singleton | 13.7247179 | 1 |
| chr1 | 18390000 | 18399999 | chr1 | 18480000 | 18489999 | 52 | 17.6783492 | 1.1804E−08 | chr1_06 | 1 | Singleton | 11.0236868 | 1 |
| chr1 | 18770000 | 18779999 | chr1 | 18790000 | 18799999 | 111 | 54.0679492 | 5.2206E−09 | chr1_07 | 1 | Singleton | 11.4079338 | 1 |
| chr1 | 24930000 | 24939999 | chr1 | 25200000 | 25209999 | 31 | 6.9555855 | 5.4986E−09 | chr1_08 | 1 | Singleton | 11.3839328 | 1 |
| chr1 | 26300000 | 26309999 | chr1 | 26320000 | 26329999 | 106 | 57.3318374 | 2.2665E−06 | chr1_09 | 1 | Singleton | 8.51531103 | 1 |
| chr1 | 27850000 | 27859999 | chr1 | 27950000 | 27959999 | 46 | 14.9454623 | 3.2917E−08 | chr1_010 | 1 | Singleton | 10.5411834 | 1 |
| chr1 | 33370000 | 33379999 | chr1 | 33450000 | 33459999 | 48 | 17.1434796 | 2.4829E−07 | chr1_011 | 1 | Singleton | 9.57843026 | 1 |
| chr1 | 34460000 | 34469999 | chr1 | 34680000 | 34689999 | 26 | 6.99962624 | 5.0207E−06 | chr1_012 | 1 | Singleton | 8.13611515 | 1 |
| chr1 | 36520000 | 36529999 | chr1 | 37000000 | 37009999 | 17 | 3.07468223 | 3.8338E−06 | chr1_013 | 1 | Singleton | 8.26482229 | 1 |
| chr1 | 36770000 | 36779999 | chr1 | 37000000 | 37009999 | 26 | 7.57042556 | 2.0162E−05 | chr1_014 | 1 | Singleton | 7.45334892 | 1 |
| chr1 | 38920000 | 38929999 | chr1 | 38990000 | 38999999 | 54 | 23.9734195 | 2.2864E−05 | chr1_015 | 1 | Singleton | 7.39136005 | 1 |
| chr1 | 43470000 | 43479999 | chr1 | 43490000 | 43499999 | 108 | 60.474515 | 8.3436E−06 | chr1_016 | 1 | Singleton | 7.89075547 | 1 |
| chr1 | 51620000 | 51629999 | chr1 | 51760000 | 51769999 | 35 | 10.783545 | 9.6822E−07 | chr1_017 | 1 | Singleton | 8.92655586 | 1 |

TABLE 4C
Representative List of Chromatin Loops and Interactions in H1 hESCs Identified by MAPS in HiChIP Data

| seqnames1 | start1 | end1 | seqnames2 | Start2 | end2 | counts | expected | fdr |
|---|---|---|---|---|---|---|---|---|
| chr1 | 1010000 | 1019999 | chr1 | 1060000 | 1069999 | 17 | 2.76770223 | 4.75E−08 |
| chr1 | 48670000 | 48679999 | chr1 | 50340000 | 50349999 | 17 | 1.5523289 | 7.67E−12 |
| chr1 | 48910000 | 48919999 | chr1 | S0340000 | 50349999 | 10 | 1.38532696 | 1.07E−05 |
| chr1 | 28780000 | 28789999 | chr1 | 28870000 | 28879999 | 21 | 3.43858931 | 1.09E−09 |
| chr1 | 17120000 | 17129999 | chr1 | 17330000 | 17339999 | 64 | 8.99920214 | 3.13E−31 |
| chr1 | 17050000 | 17059999 | chr1 | 17400000 | 17409999 | 18 | 1.71656657 | 3.56E−12 |
| chr1 | 17120000 | 17129999 | chr1 | 17400000 | 17409999 | 39 | 6.24572113 | 1.72E−17 |
| chr1 | 1780000 | 1789999 | chr1 | 1900000 | 1909999 | 19 | 2.97137042 | 3.53E−09 |
| chr1 | 1960000 | 1969999 | chr1 | 2040000 | 2049999 | 63 | 6.96388277 | 1.51E−36 |
| chr1 | 9260000 | 9269999 | chr1 | 9280000 | 9289999 | 52 | 16.014065 | 1.53E−11 |
| chr1 | 36340000 | 36349999 | chr1 | 36420000 | 36429999 | 31 | 5.4078847 | 4.04E−13 |
| chr1 | 36360000 | 36369999 | chr1 | 36420000 | 36429999 | 28 | 9.84129708 | 1.71E−05 |
| chr1 | 9620000 | 9629999 | chr1 | 9720000 | 9729999 | 40 | 5.82489617 | 2.46E−19 |
| chr1 | 6240000 | 6249999 | chr1 | 6430000 | 6439999 | 18 | 3.08969168 | 4.06E−08 |
| chr1 | 6240000 | 6249999 | chr1 | 6280000 | 6289999 | 20 | 4.98172371 | 2.62E−06 |
| chr1 | 7450000 | 7459999 | chr1 | 7560000 | 7569999 | 9 | 5.72167285 | 7.19E−05 |
| chr1 | 24070000 | 24079999 | chr1 | 24110000 | 24119999 | 22 | 6.90418806 | 3.14E−05 |
| chr1 | 6790000 | 6799999 | chr1 | 6890000 | 6899999 | 14 | 2.12117911 | 3.67E−07 |
| chr1 | 7980000 | 7989999 | chr1 | 8010000 | 8019999 | 29 | 9.24543654 | 1.72E−06 |
| chr1 | 12620000 | 12629999 | chr1 | 12650000 | 12659999 | 39 | 9.99584108 | 4.19E−11 |
| chr1 | 26280000 | 26289999 | chr1 | 26410000 | 26419999 | 31 | 11.9422468 | 3.26E−05 |
| chr1 | 26360000 | 26369999 | chr1 | 26410000 | 26419999 | 68 | 21.6038336 | 3.19E−14 |

TABLE 4D
Representative List of Chromatin Loops and Interactions in H1 hESCs Identified by HiCCUPS in In Situ HiC Data

| | | | | | | | | | | | | | expected | | | | | | | | | | |
|---|---|---|---|---|---|---|---|---|---|---|---|---|---|---|---|---|---|---|---|---|---|---|---|
| chr1 | s1 | s2 | chr2 | s1 | s2 | [symbol] | [symbol] | [symbol] | [symbol] | color | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | 10 | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

TABLE 4E
Representative List of Chromatin Loops and Interactions in H1 ESCs Identified by
MAPS in PLAC-seq H3K4me3 Data

| seq | | | seq | | | | | |
|---|---|---|---|---|---|---|---|---|
| names1 | start1 | end1 | names2 | start2 | end2 | counts | expected | fdr |
| chr1 | 770000 | 779999 | chr1 | 820000 | 829999 | 17 | 2.79646826 | 8.54E−08 |
| chr1 | 1370000 | 1379999 | chr1 | 1470000 | 1479999 | 41 | 17.5662041 | 2.18E−05 |
| chr1 | 2410000 | 2419999 | chr1 | 2580000 | 2589999 | 47 | 15.262521 | 1.64E−09 |
| chr1 | 3620000 | 3629999 | chr1 | 3670000 | 3679999 | 62 | 23.302616 | 8.11E−10 |
| chr1 | 6190000 | 6199999 | chr1 | 6410000 | 6419999 | 28 | 10.4765155 | 6.83E−05 |
| chr1 | 8020000 | 8029999 | chr1 | 8260000 | 8269999 | 16 | 3.45772741 | 7.38E−06 |
| chr1 | 9870000 | 9879999 | chr1 | 10030000 | 10039999 | 31 | 10.9875586 | 8.68E−06 |
| chr1 | 10630000 | 10639999 | chr1 | 10860000 | 10869999 | 16 | 4.04298419 | 5.00E−05 |
| chr1 | 10470000 | 10479999 | chr1 | 10880000 | 10889999 | 20 | 5.79636873 | 3.35E−05 |
| chr1 | 17540000 | 17549999 | chr1 | 17580000 | 17589999 | 48 | 21.4901002 | 1.10E−05 |
| chr1 | 20210000 | 20219999 | chr1 | 20360000 | 20369999 | 28 | 4.59233394 | 3.06E−12 |
| chr1 | 23340000 | 23349999 | chr1 | 23400000 | 23409999 | 89 | 33.7268865 | 1.33E−13 |
| chr1 | 25810000 | 25819999 | chr1 | 26020000 | 26029999 | 23 | 7.43528406 | 4.20E−05 |
| chr1 | 26530000 | 26539999 | chr1 | 26820000 | 26829999 | 29 | 10.953973 | 5.77E−05 |
| chr1 | 26530000 | 26539999 | chr1 | 26860000 | 26869999 | 28 | 9.39197866 | 9.76E−06 |
| chr1 | 27340000 | 27349999 | chr1 | 27650000 | 27659999 | 15 | 3.64643564 | 5.87E−05 |
| chr1 | 27320000 | 27329999 | chr1 | 27660000 | 27669999 | 27 | 5.43604663 | 6.68E−10 |
| chr1 | 28240000 | 28249999 | chr1 | 28500000 | 28509999 | 39 | 10.6457604 | 4.84E−10 |
| chr1 | 28640000 | 28649999 | chr1 | 28870000 | 28879999 | 34 | 7.07804962 | 7.76E−12 |
| chr1 | 29230000 | 29239999 | chr1 | 29500000 | 29509999 | 38 | 6.88999961 | 5.84E−15 |
| chr1 | 32010000 | 32019999 | chr1 | 32060000 | 32069999 | 97 | 45.0455956 | 5.96E−10 |
| chr1 | 32390000 | 32399999 | chr1 | 32610000 | 32619999 | 35 | 13.3669148 | 9.93E−06 |
| chr1 | 32390000 | 32399999 | chr1 | 32520000 | 32529999 | 42 | 13.970855 | 2.65E−08 |
| chr1 | 38940000 | 38949999 | chr1 | 38990000 | 38999999 | 75 | 35.5098982 | 1.56E−07 |
| chr1 | 42840000 | 42849999 | chr1 | 43140000 | 43149999 | 36 | 12.2340443 | 5.13E−07 |
| chr1 | 43540000 | 43549999 | chr1 | 44030000 | 44039999 | 17 | 4.65760172 | 7.54E−05 |
| chr1 | 44790000 | 44799999 | chr1 | 45300000 | 45309999 | 17 | 2.8307553 | 1.01E−07 |
| chr1 | 46300000 | 46309999 | chr1 | 46330000 | 46339999 | 148 | 58.2864792 | 9.09E−21 |
| chr1 | 46440000 | 46449999 | chr1 | 46610000 | 46619999 | 39 | 14.7583157 | 2.16E−06 |

Next, the precision of HiCAR-identified interactions was assessed. However, due to the lack of a complete list of “true interactions” in H1 hESCs, the question became whether HiCAR interactions recapitulated the known features of chromatin contacts. Based on the loop exclusion model, CTCF/Cohesin-associated loops prefer convergent CTCF motif orientations at loop anchors (Rao S S P, et al. (2014) Cell. 159:1665-1680). Thus, the CTCF motif orientation of the HiCAR interactions identified by MAPS was examined. 62.8% of HiCAR interactions harbored convergent CTCF motifs on their anchors, and this ratio was comparable to that observed by PLAC-seq (FIG. 3C, 60.3%). This result demonstrated that the precision of HiCAR in identifying interactions was comparable to PLAC-seq.

Of note, there were more in situ Hi-C loops (76.9%) anchored at the convergent CTCF motif (FIG. 3C). This difference could be due the fact that HiCCUPS used the local background model for loop calling, and therefore only identified the most significant loop summits among a cluster of loops/interactions (FIG. 3A). To further explore the regulatory role of HiCAR interactions on gene expression, whether HiCAR interactions were enriched for expression quantitative trait loci (eQTL) and their associated genes (TSS) previously identified in human pluripotent stem cells (hPSC) (DeBoever C, et al. Cell Stem Cell. 20:533-546.e7) was examined. 5.368 human iPSC eQTL-TSS pairs overlapping with HiCAR loops were observed, whereas only 3,228 eQTL-TSS pairs were expected to overlap with genomic region pairs which are randomly selected (shuffled 10,000 times) with linear distances matched to HiCAR interactions (FIG. 3D, empirical p-value <0.0001, detailed in material and Methods). The significantly enriched eQTL-TSS pairs at HiCAR interactions strongly indicated the regulatory role of HiCAR interactions on gene expression in human pluripotent stem cells.

Finally, to directly test the causal role of HiCAR interactions, three putative SOX2 enhancers were selected for perturbation analysis. As shown in FIG. 3E, two enhancers (#1 and #2) were located ˜430 KB from the SOX2 TSS and enhancer #3 was located 788 KB away from the SOX2 TSS. All three candidate enhancers were open chromatin regions that form long-range interactions with the SOX2 promoter as identified by HiCAR. The sgRNAs (Table 2, supra) were specifically direct the epigenetic silencer dCas9-KRAB to the three candidate enhancers (FIG. 3E). After introducing these CRISPR inhibition components into H1 hESCs to perturb these putative SOX2 enhancers, significant down-regulation of SOX2 mRNA expression was observed using RT-qPCR (FIG. 3F). These results showed that HiCAR was a sensitive and accurate method to identify high-confidence cis-regulatory chromatin interactions at high-resolution. More importantly, HiCAR interactions likely reflected functional communication between cis-regulatory elements and their distal target genes.

### Example 5

### The Epigenetically Poised, Bivalent, and Repressed Chromatin Sequences Exhibited Extensive Spatial Activity Comparable to the Active Chromatin Regions

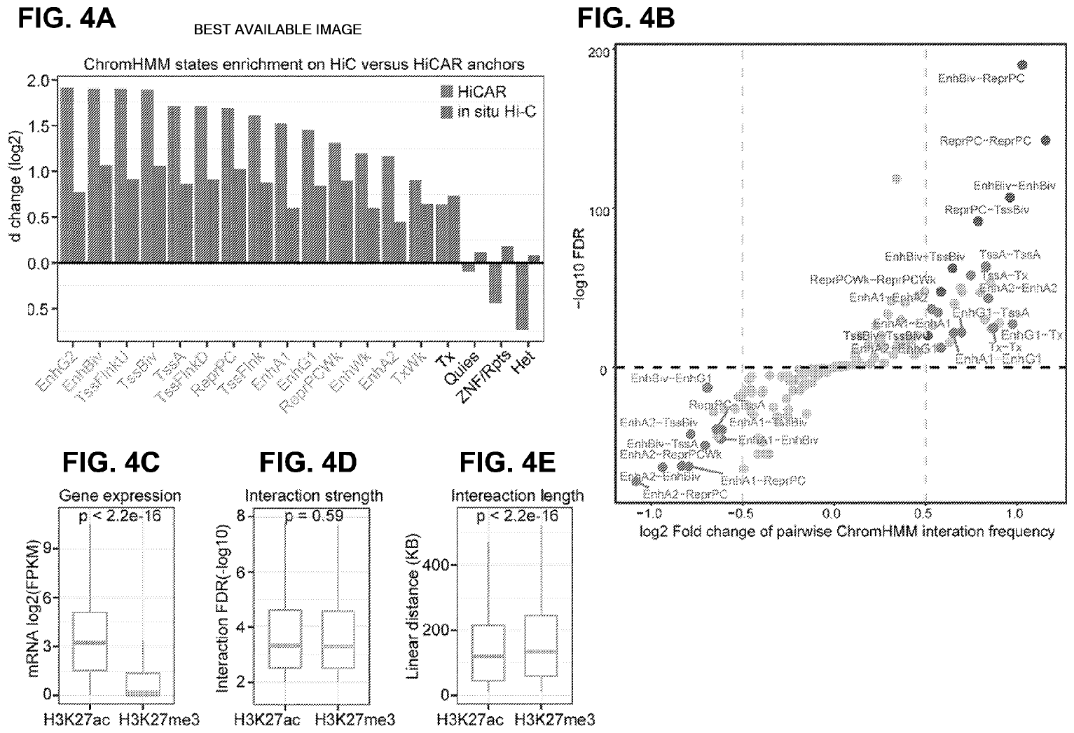

Regulatory open chromatin sequences are associated with an array of diverse epigenome signatures. Therefore, whether the HiCAR interactions could enrich cRE-interactions anchored on different chromatin states was examined. The 18 chromatin states annotation of H1 hESC defined by ChromHMM were used. Then, the enrichment fold of HiCAR interactions on each state was compared to that of HiCCUPS loops identified by H1 hESC in situ Hi-C (FIG. 4A). HiCAR interactions showed higher enrichment fold across multiple chromatin states, including enhancers, promoters, and regions associated with active. poised, bivalent, and repressed states (FIG. 4A, the chromatin states highlighted in blue text). Interestingly, compared to HiCCUPS loops, HiCAR interactions were depleted at three chromatin states—Quiescence/low (Quies), ZNF genes & repeats (ZNF/Rpts), and Heterochromatin (Het). The depletion of HiCAR interactions on these three states could be due to the lack of open chromatin regions on those sequences, as the “Quies” state lack any known marks associated with cRE, while the “ZNF/Rpts” and “Het” sequences were highly enriched for the heterochromatin mark H3K9me3 (Ernst J, et al. (2017) Nat. Protoc. 12:2478-2492). Next, how often one chromatin state was interacting with all 18 chromatin states was examined. Whether the observed interaction frequency between two chromatin states was over- or under-represented compared to the genome-wide background was determined (Table 5).

TABLE 5
Statistical Analysis of Pairwise chromHMM States Interaction Frequency

| | | ob_ | exp_ | Enrichment_ | | | |
|---|---|---|---|---|---|---|---|
| Feature1 | Feature2 | Interactions | Interactions | Ratio_log2 | Enrichment_logP | p-Value | fdr |
| EnhA1 | EnhAl | 1110 | 749.041839 | 0.567441467 | −80.35933878 | 1.26E−35 | 4.39E−35 |
| EnhA1 | EnhA2 | 1328 | 918.08754 | 0.53255152 | −85.49007448 | 7.45E−38 | 2.74E−37 |
| EnhA1 | EnhBiv | 933 | 1432.40332 | −0.618488785 | 105.46362 | −1.58E−46 | 8.58E−46 |
| EnhA1 | EnhG1 | 530 | 336.970658 | 0.653369388 | −50.45551103 | 1.22E−22 | 2.73E−22 |
| EnhA1 | EnhWk | 3863 | 3569.72186 | 0.11391001 | −15.27074834 | 2.33E−07 | 3.14E−07 |
| EnhA1 | Het | 276 | 248.978222 | 0.148648711 | −3.041853863 | 0.047746292 | 0.05366525 |
| EnhA1 | Quies | 3823 | 4559.34649 | −0.254121851 | 72.60878274 | −2.93E−32 | 9.48E−32 |
| EnhA1 | ReprPC | 760 | 1317.8384 | −0.794102148 | 146.310481 | −2.87E−64 | 3.91E−63 |
| EnhA1 | ReprPCWk | 1258 | 1705.13713 | −0.438755846 | 69.93265532 | −4.25E−31 | 1.31E−30 |
| EnhA1 | TssA | 1423 | 1298.48313 | 0.132108388 | −8.153360459 | 2.88E−04 | 3.49E−04 |
| EnhA1 | TssBiv | 829 | 1267.84122 | −0.612930072 | 91.98236111 | −1.13E−40 | 4.51E−40 |
| EnhA1 | TssFlnk | 1155 | 1366.56578 | −0.242662052 | 20.40212076 | −1.38E−09 | 1.95E−09 |
| EnhA1 | TssFlnkD | 890 | 883.878983 | 0.009956481 | −0.86214274 | 0.422256327 | 0.43178091 |
| EnhA1 | TssFlnkU | 1142 | 866.185083 | 0.398815418 | −43.74357891 | 1.01E−19 | 2.04E−19 |
| EnhA1 | Tx | 1007 | 769.033001 | 0.388946269 | −37.08657437 | 7.83E−17 | 1.44E−16 |
| EnhA1 | TxWk | 4005 | 3227.43777 | 0.311412965 | −97.38651356 | 5.08E−43 | 2.23E−42 |
| EnhA1 | ZNF_Rpts | 269 | 222.304801 | 0.275067069 | −6.666055903 | 0.001273411 | 0.00151916 |
| EnhA2 | EnhA2 | 683 | 380.705797 | 0.843209041 | −101.2573839 | 1.06E−44 | 5.14E−44 |
| EnhA2 | EnhBiv | 534 | 1022.94421 | −0.937815817 | 147.7552736 | −6.77E−65 | 1.02E−63 |
| EnhA2 | EnhG1 | 361 | 240.877619 | 0.583698487 | −28.86988872 | 2.90E−13 | 4.53E−13 |
| EnhA2 | EnhWk | 2679 | 2691.77448 | −0.006862966 | 0.90436944 | −0.404797054 | 0.41706363 |
| EnhA2 | Quies | 2913 | 3247.97586 | −0.157035207 | 21.87766522 | −3.15E−10 | 4.56E−10 |
| EnhA2 | ReprPC | 444 | 939.822005 | −1.081827871 | 168.8902224 | −4.49E−74 | 1.02E−72 |
| EnhA2 | ReprPCWk | 684 | 1217.36511 | −0.831693695 | 145.5290113 | −6.27E−64 | 7.11E−63 |
| EnhA2 | TssA | 1184 | 928.181991 | 0.351189469 | −36.05976971 | 2.18E−16 | 3.91E−16 |
| EnhA2 | TssBiv | 525 | 904.268418 | −0.784433656 | 98.56738274 | −1.56E−43 | 7.07E−43 |
| EnhA2 | TssFlnk | 921 | 975.226521 | −0.082536204 | 3.216188914 | −0.040107621 | 0.04583728 |
| EnhA2 | TssFlnkD | 635 | 632.551596 | 0.005573429 | −0.762819406 | 0.466349742 | 0.46744008 |
| EnhA2 | TssFlnkU | 921 | 632.875265 | 0.541279974 | −61.50516144 | 1.94E−27 | 5.06E−27 |
| EnhA2 | Tx | 686 | 548.330981 | 0.323161587 | −18.80880017 | 6.78E−09 | 9.51E−09 |
| EnhA2 | Tx Wk | 2767 | 2313.48648 | 0.258253975 | −47.22849739 | 3.08E−21 | 6.76E−21 |
| EnhBiv | EnhBiv | 1339 | 687.805838 | 0.961082693 | −249.2362713 | 5.73E−109 | 1.95E−107 |
| EnhBiv | EnhG1 | 201 | 325.10988 | −0.693731896 | 30.24571232 | −7.32E−14 | 1.16E−13 |
| EnhBiv | EnhWk | 2970 | 3672.44657 | −0.30627857 | 80.92704023 | −7.14E−36 | 2.56E−35 |
| EnhBiv | Het | 219 | 238.615764 | −0.123758487 | 2.242195665 | −0.106225014 | 0.11650485 |
| EnhBiv | Quies | 3424 | 4383.25574 | −0.356320153 | 127.881398 | −2.90E−56 | 2.63E−55 |
| EnhBiv | ReprPC | 2095 | 1026.6813 | 1.028961837 | −442.534306 | 6.45E−193 | 8.78E−19] |
| EnhBiv | ReprPCWk | 2141 | 1558.86554 | 0.457788298 | −104.4692669 | 4.26E−46 | 2.15E−45 |
| EnhBiv | TssA | 773 | 1260.29269 | −0.70521851 | 115.3417607 | −8.09E−51 | 5.79E−50 |
| EnhBiv | TssBiv | 1585 | 1011.54743 | 0.647918874 | −145.5813191 | 5.95E−64 | 7.11E−63 |
| EnhBiv | TssFlnk | 831 | 1295.02676 | −0.640061526 | 100.9759738 | −1.40E−44 | 6.57E−44 |
| EnhBiv | TssFlnkD | 620 | 847.924711 | −0.451667954 | 37.22312735 | −6.83E−17 | 1.27E−16 |
| EnhBiv | TssFlnkU | 573 | 868.926695 | −0.600699334 | 61.32741786 | −2.32E−27 | 5.85E−27 |
| EnhBiv | Tx | 539 | 737.935255 | −0.453208969 | 32.81484688 | −5.61E−15 | 9.08E−15 |
| EnhBiv | TxWk | 2443 | 3208.32575 | −0.393166769 | 109.6599282 | −2.37E−48 | 1.34E−47 |
| EnhG1 | EnhWk | 1018 | 873.884065 | 0.220223761 | −13.9779738 | 8.50E−07 | 1.13E−06 |
| EnhG1 | Quies | 712 | 1037.0004 | −0.542467306 | 61.49147293 | −1.97E−27 | 5.06E−27 |
| EnhG1 | ReprPCWk | 303 | 386.890185 | −0.352606335 | 12.19927789 | −5.03E−06 | 6.40E−06 |
| EnhG1 | TssA | 482 | 297.485027 | 0.696216092 | −51.51326856 | 4.25E−23 | 9.63E−23 |
| EnhG1 | TssBiv | 210 | 287.281787 | −0.452077204 | 13.85505997 | −9.61E−07 | 1.27E−06 |
| EnhG1 | TssFlnk | 425 | 309.215068 | 0.45885222 | −22.20013626 | 2.28E−10 | 3.34E−10 |
| EnhG1 | TssFlnkD | 362 | 199.705128 | 0.858118318 | −56.37740599 | 3.28E−25 | 7.69E−25 |
| EnhG1 | TssFlnkU | 383 | 205.002375 | 0.901703767 | −64.85689958 | 6.81E−29 | 1.898−28 |
| EnhG1 | Tx | 325 | 165.212676 | 0.976115334 | −63.48686142 | 2.68E−28 | 7.29E−28 |
| EnhG1 | Tx Wk | 984 | 752.86455 | 0.386267987 | −35.83829551 | 2.73E−16 | 4.82E−16 |
| EnhG2 | EnhWk | 353 | 308.084967 | 0.196339897 | −5.055676654 | 0.006373053 | 0.00753683 |
| EnhG2 | Quies | 26 | 364.50765 | −0.465411163 | 17.896284 | −1.69E−08 | 2.30E−08 |
| EnhG2 | TxWk | 362 | 266.714586 | 0.440692972 | −17.94980856 | 1.60E−08 | 2.20E−08 |
| EnhWk | EnhWk | 5415 | 5001.56704 | 0.114581163 | −21.41071692 | 5.03E−10 | 7.20E−10 |
| EnhWk | Het | 691 | 643.174486 | 0.103475533 | −3.467162632 | 0.031205447 | 0.0359656 |
| EnhWk | Quies | 11074 | 11376.2346 | −0.038846688 | 7.497552559 | −5.54E−04 | 6.67E−04 |
| EnhWk | ReprPC | 2545 | 3394.37258 | −0.415479275 | 128.1975373 | −2.11E−56 | 2.05E−55 |
| EnhWk | ReprPCWk | 4073 | 4295.25013 | −0.076650333 | 8.655865825 | −1.74E−04 | 2.15E−04 |
| EnhWk | TssA | 3106 | 3341.05917 | −0.105247704 | 11.47140745 | −1.04E−05 | 1.31E−05 |
| EnhWk | TssBiv | 2632 | 3263.94325 | −0.310456483 | 73.37906614 | −1.35E−32 | 4.49E−32 |
| EnhWk | TssFlnk | 3081 | 3465.85413 | −0.169812255 | 26.70198385 | −2.53E−12 | 3.87E−12 |
| EnhWk | TssFlnkD | 2205 | 2212.03065 | −0.004592722 | 0.810334632 | −0.444709228 | 0.45134668 |
| EnhWk | TssFlnkU | 2336 | 2290.64994 | 0.028283275 | −1.782449898 | 0.168225507 | 0.180147 |
| EnhWk | Tx | 2347 | 1983.91405 | 0.242468318 | −35.80706229 | 2.81E−16 | 4.90E−16 |
| EnhWk | TxWk | 8288 | 7533.62269 | 0.137680223 | −46.99676707 | 3.89E−21 | 8.31E−21 |
| EnhWk | ZNF_Rpts | 601 | 574.038699 | 0.066216992 | −2.013310976 | 0.133545775 | 0.1452978 |
| Het | Quies | 781 | 755.947366 | 0.047036761 | −1.694612324 | 0.18367042 | 0.19514982 |
| Het | ReprPCWk | 260 | 283.866716 | −0.126702078 | 2.517835384 | −0.08063396 | 0.08988704 |
| Het | TssA | 211 | 219.956551 | −0.059975571 | 1.25045791 | −0.286373633 | 0.30053084 |
| Het | TssBiv | 209 | 210.847747 | −0.012698662 | 0.760484109 | −0.46744008 | 0.46744008 |
| Het | TssFlnk | 219 | 228.069036 | −0.05853972 | 1.247325323 | −0.28727213 | 0.30053084 |
| Het | TxWk | 525 | 556.958498 | −0.085252406 | 2.418820939 | −0.089026523 | 0.09843583 |
| Quies | Quies | 8859 | 6997.47442 | 0.340309549 | −276.4399869 | 8.78E−121 | 3.98E−119 |
| Quies | ReprPC | 3106 | 4026.68484 | −0.374534731 | 127.6987209 | −3.48E−56 | 2.96E−55 |
| Quies | ReprPCWk | 4769 | 5197.0304 | −0.124000714 | 23.06210981 | −9.64E−11 | 1.43E−10 |
| Quies | TssA | 3387 | 4027.55062 | −0.249894736 | 61.84466308 | −1.38E−27 | 3.69E−27 |
| Quies | TssBiv | 3475 | 3872.75651 | −0.156347819 | 25.78112817 | −6.36E−12 | 9.50E−12 |
| Quies | TssFlnk | 3887 | 4164.49559 | −0.099484658 | 12.78817783 | −2.79E−06 | 3.62E−06 |
| Quies | TssFlnkD | 2312 | 2704.5269 | −0.226234848 | 34.61819913 | −9.24E−16 | 1.57E−15 |
| Quies | TssFlnkU | 2078 | 2773.97521 | −0.416759241 | 104.5551178 | −3.91E−46 | 2.05E−45 |
| Quies | Tx | 1871 | 2353.7452 | −0.331148595 | 59.03157342 | −2.31E−26 | 5.60E−26 |
| Quies | TxWk | 9170 | 10175.3239 | −0.150081073 | 68.36494771 | −2.04E−30 | 6.03E−30 |
| Quies | ZNF_Rpts | 633 | 678.090183 | −0.099271658 | 3.191069828 | −0.041127848 | 0.04661156 |
| ReprPC | ReprPC | 1293 | 580.164958 | 1.15618721 | −332.8243064 | 2.86E−145 | 1.94E−143 |
| ReprPC | ReprPCWk | 1985 | 1409.65489 | 0.493797 | −111.2263302 | 4.95E−49 | 3.37E−48 |
| ReprPC | TssA | 743 | 1160.07829 | −0.642788056 | 91.1533492 | −2.59E−40 | 1.00E−39 |
| ReprPC | TssBiv | 1643 | 952.007275 | 0.787287976 | −214.6037004 | 6.29E−94 | 1.71E−92 |
| ReprPC | TssFlnk | 859 | 1193.01327 | −0.47388005 | 56.12111418 | −4.24E−25 | 9.76E−25 |
| ReprPC | TssFlnkD | 544 | 780.281359 | −0.520387783 | 43.55316233 | −1.22E−19 | 2.43E−19 |
| ReprPC | TssFlnkU | 507 | 799.654848 | 0.657391682 | 65.60021459 | −3.24E−29 | 9.17E−29 |
| ReprPC | Tx | 483 | 677.750428 | −0.48873093 | 34.31865982 | −1.25E−15 | 2.09E−15 |
| ReprPC | TxWk | 2093 | 2949.37677 | −0.494837822 | 150.3393787 | −5.11E−66 | 9.93E−65 |
| ReprPCWk | ReprPCWk | 1460 | 973.942658 | 0.584059629 | −111.0700428 | 5.79E−49 | 3.75E−48 |
| ReprPCWk | TssA | 1097 | 1503.42439 | −0.454688793 | 65.63684866 | −3.12E−29 | 9.03E−29 |
| ReprPCWk | TssBiv | 1682 | 1398.33586 | 0.266466789 | −30.77276995 | 4.32E−14 | 6.91E−14 |
| ReprPCWk | TssFlnk | 1330 | 1544.77901 | −0.215974221 | 18.76790252 | −7.07E−09 | 9.81E−09 |
| ReprPCWk | TssFlnkD | 890 | 1007.10612 | −0.178338467 | 9.466236873 | −7.74E−05 | 9.66E−05 |
| ReprPCWk | TssFlnkU | 791 | 1035.31985 | −0.388326939 | 34.8928501 | −7.02E−16 | 1.21E−15 |
| ReprPCWk | Tx | 806 | 878.149899 | −0.123687389 | 4.994801669 | −0.006773064 | 0.00794083 |
| ReprPCWk | TxWk | 3354 | 3813.40668 | −0.185197707 | 34.21900595 | −1.38E−15 | 2.28E−15 |
| ReprPCWk | ZNF_Rpts | 222 | 253.321454 | −0.190409587 | 3.71812622 | −0.02427942 | 0.02822223 |
| TssA | TssA | 1039 | 584.518483 | 0.829875105 | −148.9924313 | 1.97E−65 | 3.34E−64 |
| TssA | TssBiv | 837 | 1098.52297 | −0.39226551 | 37.56183065 | −4.87E−17 | 9.19E−17 |
| TssA | TssFlnk | 1366 | 948.804084 | 0.525775359 | −85.88894073 | 5.00E−38 | 1.89E−37 |
| TssA | TssFlnkD | 961 | 700.916488 | 0.455293868 | −46.99106969 | 3.91E−21 | 8.31E−21 |
| TssA | TssFlnkU | 1048 | 645.298231 | 0.69960074 | −110.7117718 | 8.29E−49 | 5.12E−48 |
| TssA | Tx | 1137 | 677.209237 | 0.747558699 | −135.2063709 | 1.91E−59 | 2.00E−58 |
| TssA | TxWk | 3575 | 2914.12585 | 0.29488006 | −78.20018202 | 1.09E−34 | 3.71E−34 |
| TssA | ZNF_Rpts | 201 | 196.428894 | 0.033188341 | −0.963978868 | 0.381372432 | 0.39592863 |
| TssBiv | TssBiv | 766 | 537.095842 | 0.512164839 | −46.64305523 | 5.54E−21 | 1.16E−20 |
| TssBiv | TssFlnk | 986 | 1097.98686 | −0.155201232 | 8.211555555 | −2.71E−04 | 3.33E−04 |
| TssBiv | TssFlnkD | 626 | 744.920287 | −0.250923395 | 12.54546043 | −3.56E−06 | 4.57E−06 |
| TssBiv | TssFlnkU | 542 | 766.642795 | −0.500261682 | 40.10489731 | −3.83E−18 | 7.54E−18 |
| TssBiv | Tx | 535 | 652.055801 | −0.28545654 | 13.73081082 | −1.09E−06 | 1.42E−06 |
| TssBiv | TxWk | 2369 | 2835.73958 | −0.25944685 | 46.219118 | −8.46E−21 | 1.74E−20 |
| TssFlnk | TssFlnk | 991 | 628.484802 | 0.6570072 | −93.5879959 | 2.27E−41 | 9.34E−41 |
| TssFlnk | TssFlnkD | 952 | 712.51592 | 0.418039324 | −39.97936781 | 4.34E−18 | 8.43E−18 |
| TssFlnk | TssFlnkU | 983 | 776.162851 | 0.340832033 | −28.68830032 | 3.47E−13 | 5.37E−13 |
| TssFlnk | Tx | 1135 | 702.940909 | 0.691216975 | −117.2475459 | 1.20E−51 | 9.08E−51 |
| TssFlnk | TxWk | 3436 | 3016.10858 | 0.188041668 | −32.91338142 | 5.08E−15 | 8.32E−15 |
| TssFlnkD | TssFlnkD | 408 | 263.402141 | 0.631302081 | −37.06290559 | 8.01E−17 | 1.45E−16 |
| TssFlnkD | TssFlnkU | 668 | 507.463431 | 0.396544242 | −26.11895446 | 4.54E−12 | 6.85E−12 |
| TssFlnkD | Tx | 822 | 454.686736 | 0.854265474 | −124.3541254 | 9.86E−55 | 7.88E−54 |
| TssFlnkD | TxWk | 2329 | 1950.82536 | 0.255626008 | −39.10125115 | 1.04E−17 | 2.00E−17 |
| TssFlnkU | TssFlnkU | 489 | 276.165798 | 0.824299805 | −70.23967377 | 3.13E−31 | 9.89E−31 |
| TssFlnkU | Tx | 813 | 466.682997 | 0.800812447 | −109.6842171 | 2.32E−48 | 1.34E−47 |
| TssFlnkU | TxWk | 2634 | 2012.38986 | 0.388345521 | −95.18488155 | 4.59E−42 | 1.95E−41 |
| Tx | Tx | 362 | 197.948393 | 0.870865342 | −57.83581798 | 7.62E−26 | 1.82E−25 |
| Tx | TxWk | 2158 | 1675.33739 | 0.365243201 | −69.54628751 | 6.26E−31 | 1.89E−30 |
| TxWk | TxWk | 4399 | 3751.89528 | 0.229556039 | −60.98210042 | 3.28E−27 | 8.11E−27 |
| TxWk | ZNF_Rpts | 518 | 495.804924 | 0.063179499 | −1.810545382 | 0.163564907 | 0.17654625 |

Interestingly, the chromatin regions associated with similar epigenome states (epigenetically “active” states versus “inactive”” states, such as repressive/poised/repressed) tended to interact with each other (FIG. 48 with blue dots denoting the “inactive-inactive” interaction” and red dots denoting the “active-active” interaction). On the contrary, the HiCAR interactions connecting the “active” versus “inactive” chromatin states were significantly under-represented (FIG. 4B, purple dots). These results indicated that the spatial proximity of cREs played a role in facilitating the coordinated epigenomic modification of cis-regulatory sequences.

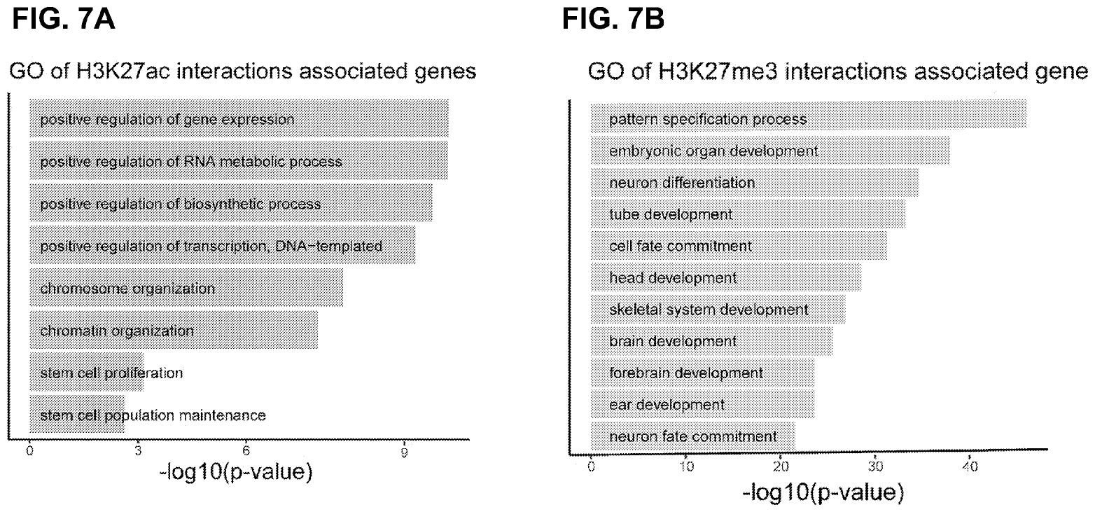

Intrigued by the observation that both “active-to-active” and “inactive-to-inactive” interactions are significantly enriched among the HiCAR interactions (FIG. 4B), the interactions anchored on the “active” versus “inactive” (poised/bivalent/repressed) chromatin states were directly compared. In ChromHMM, histone H3K27me3 modification is the common histone mark to annotate the poised, bivalent, and repressed chromatin states, while the H3K27ac mark is used to denote transcriptionally active chromatin regions. 14,845 and 10,287 HiCAR interactions with at least one anchor overlapped with H1 hESC H3K27ac or H3K27me3 ChIP-seq peaks, respectively, were selected. The interactions overlapped with both H3K27ac and H3K27me3 peaks were excluded from the following analysis. Notably, using HiCAR, the two types of interactions were captured from one single assay independent of antibody-specific ChIP enrichment, and therefore can be directly compared in terms of their numbers, interaction strength/confidence, and transcriptional/enhancer activity. As expected, genes with promoters located on H3K27ac anchors. had significantly higher mRNA expression levels compared with genes with promoters located on H3K27me3 anchors (FIG. 4C, Wilcoxon rank-sum, p<2.2e-16). Interestingly, when the interaction strength quantified by −log 10 FDR (output from MAPS) was compared between the two types of interactions, the H3K27me3-anchored interactions showed a similar distribution of FDR, which were indistinguishable from the interactions anchored on H3K27ac peaks (FIG. 4D, Wilcoxon rank-sum, p=0.59). The H3K27me3-anchored interactions showed significantly longer linear genomic distance (median distance 145 KB) than the 113K27ac-anchored interactions (median distance 125 KB) (FIG. 4E. Wilcoxon rank-sum, p <2.2e-16). Furthermore, through gene ontology (GO) analysis, the genes with promoters located on the H3K27ac-anchored interactions were enriched for GO terms related to transcription, metabolic, chromatin organization, and stem cell proliferation/maintenance (FIG. 7A), while genes associated with H3K27me3 anchors were enriched for GO terms important for lineage specific tissue and organ differentiation/development (FIG. 78). This GO enrichment analysis indicated that the two types of interactions can play different roles in regulating gene expression in distinct biological processes. In summary, these results showed that the epigenetically “inactive” (poised, bivalent, and repressed) cREs tend to form massive, long-range, and significant chromatin interactions that are comparable to the interactions associated with “active” cREs.

### Example 6

### Identification of Epigenome Features Important for the Spatial Interaction Activity of Cis-Regulatory Sequences in H1 ESC

The high-resolution (5 KB bin) cRE-contact map and the rich public epigenome datasets available for H1 hESC (Table 1. supra) provided an opportunity to study the epigenome features important for the spatial activity of cREs. To probe this question, a method described previously ^{35, 36} was employed to calculate the cumulative interactive score (sum of −log 10 FDR) of each HiCAR interaction anchor (5 KB bin) (Table 6A, detailed supra).

Each of Tables 6A-6D are representative of the data generated in the analysis. Each of Tables 6A-6D represents a “snapshot” of the expansive volume of data generated during an analysis. As disclosed supra, HiCARTools or NF-Core/HiCAR is a bioinformatics best-practice analysis pipeline for processing these data.

TABLE 6A
HiCAR Anchor Cumulative Interactive Score

| seqnames | start | end | strand | score | type |
|---|---|---|---|---|---|
| chr1 | 625000 | 629999 | * | 130.61 | hotspot |
| chr1 | 630000 | 634999 | * | 130.61 | hotspot |
| chr1 | 1065000 | 1069999 | * | 6.82 | regular |
| chr1 | 1070000 | 1074999 | * | 6.82 | regular |
| chr1 | 1115000 | 1119999 | * | 27.71 | regular |
| chr1 | 1120000 | 1124999 | * | 34.19 | regular |
| chr1 | 1125000 | 1129999 | * | 31.16 | regular |
| chr1 | 1230000 | 1234999 | * | 5.81 | regular |
| chr1 | 1250000 | 1254999 | * | 7.82 | regular |
| chr1 | 1260000 | 1264999 | * | 7.93 | regular |
| chr1 | 1290000 | 1294999 | * | 19.44 | regular |
| chr1 | 1640000 | 1644999 | * | 18.30 | regular |
| chr1 | 1645000 | 1649999 | * | 18.30 | regular |
| chr1 | 1655000 | 1659999 | * | 3.07 | regular |
| chr1 | 1665000 | 1669999 | * | 3.63 | regular |
| chr1 | 1710000 | 1714999 | * | 18.30 | regular |
| chr1 | 1720000 | 1724999 | * | 3.07 | regular |
| chr1 | 1730000 | 1734999 | * | 3.63 | regular |
| chr1 | 1780000 | 1784999 | * | 7.87 | regular |
| chr1 | 1785000 | 1789999 | * | 17.79 | regular |
| chr1 | 1790000 | 1794999 | * | 13.34 | regular |
| chr1 | 1905000 | 1909999 | * | 8.89 | regular |
| chr1 | 1940000 | 1944999 | * | 10.49 | regular |
| chr1 | 1945000 | 1949999 | * | 12.95 | regular |
| chr1 | 1950000 | 1954999 | * | 9.65 | regular |
| chr1 | 1955000 | 1959999 | * | 12.10 | regular |
| chr1 | 1960000 | 1964999 | * | 9.63 | regular |
| chr1 | 2030000 | 2034999 | * | 2.06 | regular |
| chr1 | 2045000 | 2049999 | * | 20.53 | regular |
| chr1 | 2185000 | 2189999 | * | 10.50 | regular |

TABLE 6B
HiCAR GO Term Enrichment on Interaction Hotspots

| id | ID | Description | GeneRatio | BgRatio | pvalue | p.adjust | qvalue | geneID | Count |
|---|---|---|---|---|---|---|---|---|---|
| 1 | GO: | nucleosome | 27/363 | 135/17913 | 1.87E−19 | 7.62E−16 | 6.66E−16 | HIST1H1T, HIST1H2BC, HIST1H1E, | 27 |
| | 0006334 | assembly | | | | | | HIST1H2BE, HIST1H4D, HIST1H2BF, | |
| | | | | | | | | HIST1H3D, HIST1H4E, HIST1H2BG, | |
| | | | | | | | | HIST1H3E, HIST1H1D, HIST1H4F, | |
| | | | | | | | | HIST1H2BH, HIST1H3F, HIST1H4L, | |
| | | | | | | | | HIST1H2BJ, HIST1H3H, HIST1H2BL, | |
| | | | | | | | | HIST1H2BM, HIST1H4J, HIST1H2BN, | |
| | | | | | | | | HIST1HAK, HIST1H1B, HIST1H3I, | |
| | | | | | | | | HIST1H4L, HIST1H2BO, HIST1H3J | |
| 2 | GO: | chromatin | 28/363 | 153/17913 | 4.84E−19 | 9.86E−16 | 8.61E−16 | HIST1H1T, HIST1H2BC, HISTTH1EB, | 28 |
| | 0031497 | assembly | | | | | | HIST1H2BE, HIST1H4D, HIST1H2BF, | |
| | | | | | | | | HIST1H3D, HIST1H4E, HIST1HI2BG, | |
| | | | | | | | | HIST1H3E, HIST1H1D, HIST1H4F, | |
| | | | | | | | | HIST1H2BH, HIST1H3F, HIST1H4L, | |
| | | | | | | | | HIST1H2BJ, HIST1H3H, HIST1H2BL, | |
| | | | | | | | | HIST1H2BM, HIST1H4J, HIST1H2BN, | |
| | | | | | | | | HIST1H4K, HIST1H1B, | |
| | | | | | | | | HIST1HSI, HIST1H4L, | |
| | | | | | | | | HIST1H2BO, HIST1H3J, CDKN2A | |
| 3 | GO: | chromatin | 29/363 | 178/17913 | 3.10E−18 | 4.22E−15 | 3.69E−15 | PADI2, HIST1H1T, HIST1H2BC, | 29 |
| | 0006333 | assembly or | | | | | | HIST1H1B, HIST1H2BE, | |
| | | disassembly | | | | | | HIST1H4D, HIST1H2BF, | |
| | | | | | | | | HIST1H3D, HIST1H4E, HIST1H2BG, | |
| | | | | | | | | HIST1HSE, HIST1H1D, HIST1H4F, | |
| | | | | | | | | HIST1H2BH, HIST1H3F, HIST1H4I, | |
| | | | | | | | | HIST1H2BJ, HIST1H3H, HIST1H2BL, | |
| | | | | | | | | HIST1H2BM, HIST1H4J, HIST1H2BN, | |
| | | | | | | | | HIST1H4K, HIST1H1B, | |
| | | | | | | | | HIST1H3I, HIST1H4L, | |
| | | | | | | | | HIST1H2BO, HIST1H3J, CDKN2A | |
| 4 | GO: | nucleosome | 27/363 | 165/17913 | 4.16E−17 | 4.24E−14 | 3.71E−14 | HIST1H1T, HIST1H2BC, HIST1H1E, | 27 |
| | 0034728 | organization | | | | | | HIST1H2BE, HIST1H4D, HIST1H2BF, | |
| | | | | | | | | HIST1H3D, HIST1H4E, HIST1H2BG, | |
| | | | | | | | | HIST1H3E, HIST1H1D, HIST1H4F, | |
| | | | | | | | | HIST1H2BH, HIST1H3F, HIST1H4I, | |
| | | | | | | | | HIST1H2BJ, HIST1H3H, HIST1H2BL, | |
| | | | | | | | | HIST1H2BM, HIST1H4J, HIST1H2BN, | |
| | | | | | | | | HIST1H4K, HIST1H1B, | |
| | | | | | | | | HIST1H3I, HIST1H4L, | |
| | | | | | | | | HIST1H2BO, HIST1H3J | |
| 5 | GO: | protein-DNA | 29/363 | 210/17913 | 3.07E−16 | 2.08E−13 | 1.82E−13 | HIST1H1T, HIST1H2BC, HIST1H1E, | 29 |
| | 0065004 | complex | | | | | | HIST1H2BE, HIST1H4D, HIST1H2BF, | |
| | | assembly | | | | | | HIST1H3D, HIST1H4E, HIST1H2BG, | |
| | | | | | | | | HIST1H3E, HIST1H1D, HIST1H4F, | |
| | | | | | | | | HIST1H2BH, HIST1H3F, HIST1H4I, | |
| | | | | | | | | HIST1H2BJ, HIST1H3H, HIST1H2BL, | |
| | | | | | | | | HIST1H2BM, HIST1H4J, HIST1H2BN, | |
| | | | | | | | | HIST1H4K, HIST1H1B, | |
| | | | | | | | | HIST1H3I, HIST1H4L, | |
| | | | | | | | | HIST1H2BO, HIST1H3J, ATF7IP, UBTF | |
| 6 | GO: | DNA | 28/363 | 194/17913 | 3.16E−16 | 2.08E−13 | 1.82E−13 | HIST1H1T, HIST1H2BC, HIST1H1B, | 28 |
| | 0006323 | packaging | | | | | | HIST1H2BE, HIST1H4D, HIST1H2BF, | |
| | | | | | | | | HIST1H3D, HIST1H4E, HIST1H2BG, | |
| | | | | | | | | HIST1H3E, HIST1B1D, HIST1H4F, | |
| | | | | | | | | HIST1H2BH, HIST1H3F, HIST1H4I, | |
| | | | | | | | | HIST1H2BJ, HIST1H3H, HIST1B2BL, | |
| | | | | | | | | HIST1H2BM, HIST1H4J, HIST1H2BN, | |
| | | | | | | | | HIST1H4K, HIST1H1B, | |
| | | | | | | | | HIST1H3I, HIST1H4L, | |
| | | | | | | | | HIST1H2BO, HIST1H3J, CDKN2A | |
| 7 | GO: | positive | 76/363 | 1368/17913 | 3.57E−16 | 2.08E−13 | 1.82E−13 | PADI2, POU3F1, JUN, RNASEL, ELF3, | 76 |
| | 0045893 | regulation of | | | | | | IRF2BP2, SIX3, SIX2, | |
| | | transcription, | | | | | | MEIS1, PCBP1, HOXD3, | |
| | | DNA- | | | | | | HOXD4, FZD7, FZD5, | |
| | | templated | | | | | | IHH, PAX3, PHOX2B, | |
| | | | | | | | | FGF2, MAML3, HAND2, | |
| | | | | | | | | HEXB, NEUROG1, | |
| | | | | | | | | HAND1, FOXI1, PIM1, | |
| | | | | | | | | TAF8, VEGFA, NR2E1, | |
| | | | | | | | | IL6, HOXA1, HOXA4, | |
| | | | | | | | | HOXA5, EN2, KLF10, | |
| | | | | | | | | CDKN2B, NR6A1, GDF2, | |
| | | | | | | | | PAX2, TLX1, TNN12, | |
| | | | | | | | | IGF2, MYOD1, PRDM11, | |
| | | | | | | | | BCL9L, POU2F3, | |
| | | | | | | | | BARX2, ATF71P, HOXC11, | |
| | | | | | | | | HOXC4, GL11, | |
| | | | | | | | | TBX5, GSX1, PDX1, CDX2, | |
| | | | | | | | | ZIC2, PAX9, SIX1, | |
| | | | | | | | | FOS, IRF2BPL, MEIS2, | |
| | | | | | | | | HAS3, FOXF1, FOXC2, | |
| | | | | | | | | ETV4, SOST, ATXN7L3, | |
| | | | | | | | | UBTF, HOXB2, | |
| | | | | | | | | HOXB4, HOXB3, HOXB5, | |
| | | | | | | | | PHB, DLX3, VEZF1, | |
| | | | | | | | | CEBPB, TFAP2C | |

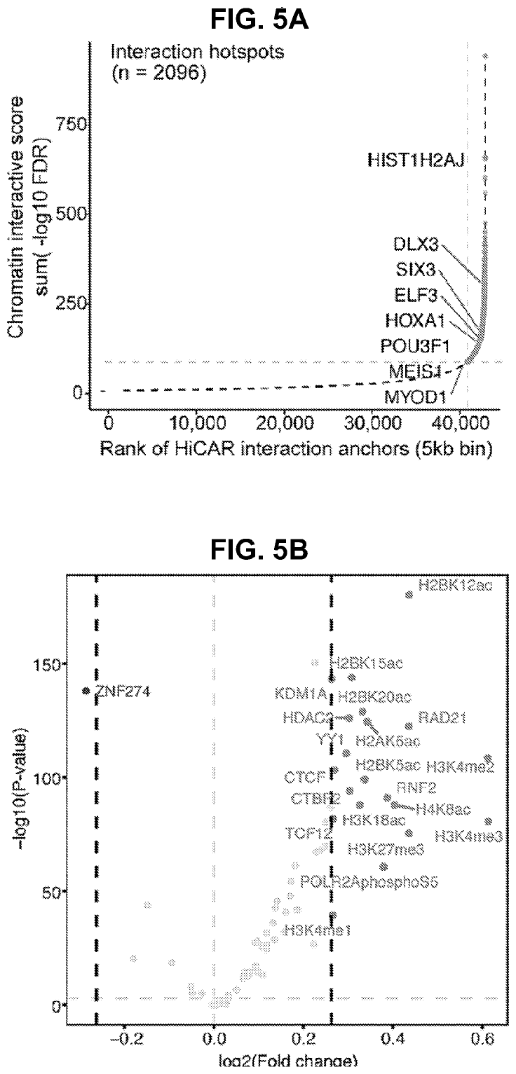

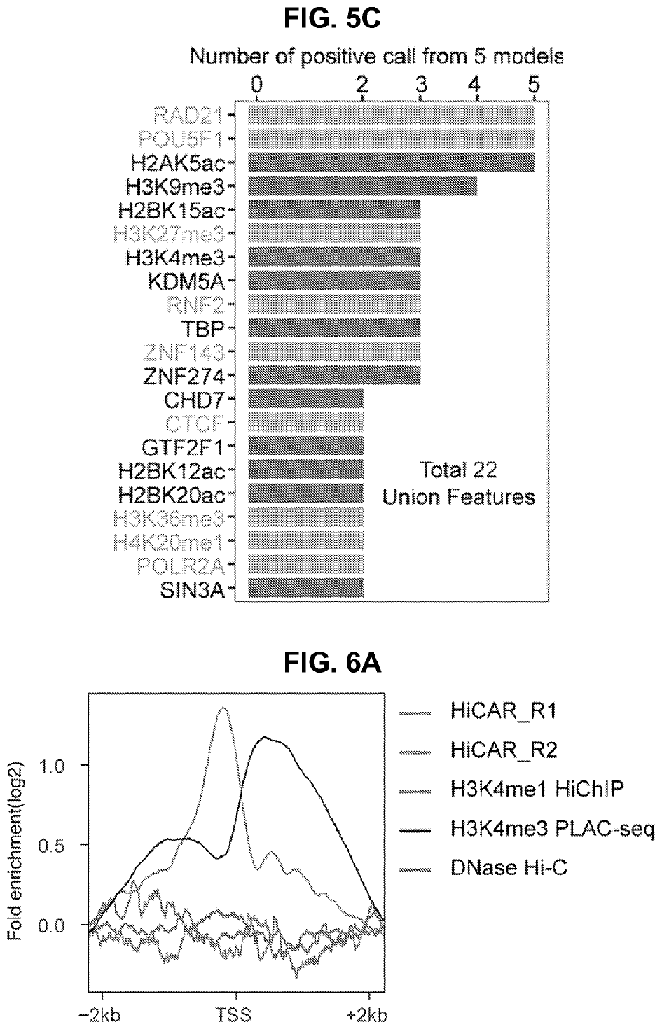

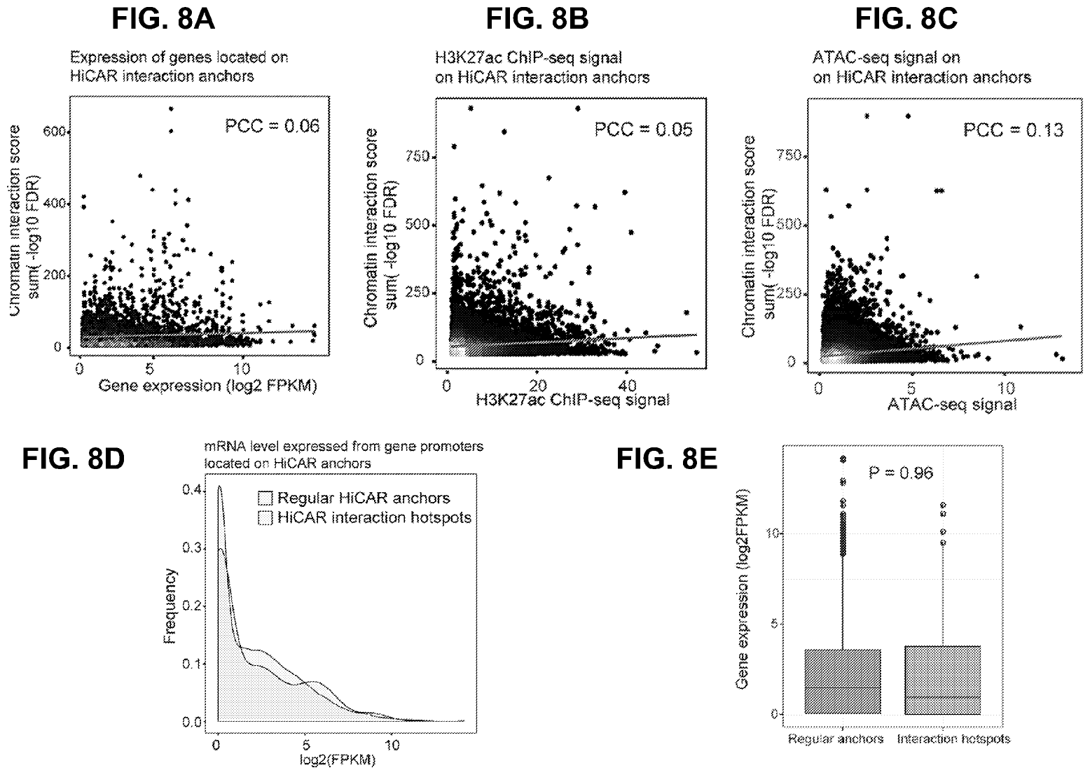

Interestingly, when this cumulative interactive score was compared with gene expression (FIG. 5A, mRNAs expressed from the gene promoters overlapped with anchors), enhancer activity (FIG. 8B, H3K27ac ChIP-seq signal on anchors), and chromatin accessibility (FIG. 5C, ATAC-seq signal on anchors), the spatial interaction activity of cREs exhibited very weak Pearson correlation coefficients with gene expression (PCC=0.06), enhancer activity (PCC 0.05) and chromatin accessibility (PCC=0.13). The question became—what chromatin epigenome features were important for the spatial activity of cREs? To address this question, the cREs associated with high-level chromatin interaction activity were identified. All 42,463 anchors based on their cumulative interactive score were ranked, and 2,096 anchors (FIG. 5A, red dots) with extremely high-level spatial interaction activity compared to other anchors (Table 6A, detailed in material and methods) were identified. Consistent with the observation that the spatial activity of cREs exhibited only weak or no correlation with transcriptional activity (FIG. 5A), the mRNA levels of the genes with promoters located on the 2,096 interaction hotspots were very similar to those of genes with promoters overlapped with regular HiCAR anchors (FIG. 5D and FIG. 5E, Wilcoxon rank-sum p=0.96).

Next, to determine the epigenome features associated with these interaction hotspots, the public ChIP-seq datasets generated from H1 hESCs (Table 1, supra) including 26 histone mark and 49 TF binding were analyzed. 9 proteins (KDM1A, HDAC2, RAD21, YY1, CTCF, CTBP2, RNF2, TCF12, and RNA Pol2) and 11 histone marks (H2BK12ac, H12BK15, H2BK20ac, H2AK5ac, H2BK5ac, H3K4mel, H3K4m2, H3K4me3, H3K27me3, H4K8ac, and H3K18ac) that are significantly enriched on the cRE-interaction hotspots were identified (FIG. 5B, red dots, fold change >1.2, FDR <0.05: detailed in Table 7). 7 of these 20 enriched histone marks and TF binding signatures (RAD21, YY1, CTCF, RNF2, RNA Pol2, H3K4mel, and H3K27me3) were known to play important roles in regulating 3D chromatin, while the involvement of the other features in genome organization remains large unexplored. Interestingly, ZNF274, a transcriptional repressor important for the establishment and maintenance of the heterochromatin mark H3K9me3, was depleted on the open chromatin interaction hotspots compared to regular HiCAR anchors (FIG. 5B, blue dot).

TABLE 7
Statistical Analysis of Ch1P-seq Sgnals Enrichment on
HiCAR Interaction Hotspots Versus Regular Anchors

| | log2(fold) (Hotspots/ | | |
|---|---|---|---|
| TF | regular anchors) | t.test.pvalue | FDR |
| H3K4me3 | 0.612857322 | 2.56E−36 | 9.15E−36 |
| H3K4me2 | 0.611628682 | 1.15E−48 | 7.83E−48 |
| H2BK12ac | 0.437102169 | 8.29E−81 | 6.22E−79 |
| RAD21 | 0.436265823 | 8.38E−55 | 6.99E−54 |
| H3K27me3 | 0.436084028 | 5.80E−34 | 1.89E−33 |
| H4K8ac | 0.403316713 | 1.58E−39 | 6.97E−39 |
| RNF2 | 0.387087917 | 5.99E−41 | 3.00E−40 |
| POLR2AphosphoS5 | 0.379297654 | 1.68E−27 | 4.49E−27 |
| H2AK5ac | 0.342710363 | 1.06E−55 | 9.93E−SS |
| H2BK5ac | 0.337344588 | 1.70E−44 | 9.81E−44 |
| H2BK20ac | 0.332100444 | 9.50E−58 | 1.19E−56 |
| H3K18ac | 0.326069338 | 1.72E−39 | 7.16E−39 |
| H2BK15ac | 0.308269872 | 1.41E−64 | 3.52E−63 |
| CTBP2 | 0.304114252 | 2.59E−42 | 1.39E−41 |
| HDAC2 | 0.302392799 | 1.75E−56 | 1.88E−55 |
| YY1 | 0.295859447 | 1.55E−49 | 1.16E−48 |
| CTCF | 0.269508857 | 2.35E−46 | 1.47E−45 |
| TCF12 | 0.266432621 | 7.56E−37 | 2.84E−36 |
| H3K4mel | 0.265813553 | 3.64E−18 | 7.38E−18 |
| KDM1A | 0.263466488 | 3.38E−64 | 6.35E−63 |
| SIN3A | 0.26149558 | 4.33E−39 | 1.71E−38 |
| SP1 | 0.250664842 | 1.41E−31 | 4.40E−31 |
| H4K91ac | 0.250405051 | 4.3SE−36 | 1.48E−35 |
| TBP | 0.2396351 | 1.03E−30 | 3.09E−30 |
| TAF1 | 0.234169785 | 1.83E−24 | 4.57E−24 |
| GABPA | 0.233148959 | 1.35E−40 | 6.32E−40 |
| RBBPS | 0.229546917 | 2.11E−30 | 6.09E−30 |
| 4-Oct | 0.225588561 | 1.30E−67 | 4.87E−66 |
| POLR2A | 0.222867923 | 1.48E−12 | 2.52E−12 |
| SAP30 | 0.18662023 | 3.35E−19 | 7.17E−19 |
| ZNF143 | 0.18267176 | 9.49E−28 | 2.64E−27 |
| H3K4ac | 0.17276966 | 9.65E−25 | 2.49E−24 |
| NANOG | 0.170888613 | 6.85E−22 | 1.66E−21 |
| JUND | 0.160570302 | 9.09E−19 | 1.89E−18 |
| H4K20me1 | 0.158668975 | 6.19E−15 | 1.19E−14 |
| H2BK120ac | 0.141790576 | 5.77E−21 | 1.3SE−20 |
| CHD2 | 0.137503618 | 1.26E−19 | 2.78E−19 |
| USF1 | 0.135518709 | 1.81E−13 | 3.31E−13 |
| H3K56ac | 0.131941717 | 1.08E−16 | 2.12E−16 |
| PHF8 | 0.118212506 | 1.94E−12 | 3.23E−12 |
| TAF7 | 0.117778781 | 1.37E−11 | 2.14E−11 |
| H3K23me2 | 0.117619947 | 9.48E−15 | 1.78E−14 |
| H3K9ac | 0.109724463 | 1.21E−06 | 1.57E−06 |
| BACH1 | 0.109160201 | 2.94E−12 | 4.79E−12 |
| CHD7 | 0.107456863 | 5.47E−12 | 8.73E−12 |
| H4K5ac | 0.100814521 | 4.45E−07 | 6.07E−07 |
| SUZ12 | 0.098716883 | 3.82E−07 | 5.31E−07 |
| ATF3 | 0.097496681 | 4.60E−13 | 8.22E−13 |
| CHD1 | 0.093550585 | 8.06E−08 | 1.16E−07 |
| EP300 | 0.092895064 | 2.49E−08 | 3.66E−08 |
| USF2 | 0.091837915 | 6.67E−13 | 1.16E−12 |
| RXRA | 0.07938293 | 3.69E−07 | 5.23E−07 |
| EGR1 | 0.076816717 | 2.07E−06 | 2.63E−06 |
| BRCA1 | 0.068588272 | 4.53E−07 | 6.07E−07 |
| H3K14ac | 0.068047624 | 7.26E−07 | 9.56E−07 |
| GTF2F1 | 0.065741176 | 6.25E−06 | 7.81E−06 |
| MYC | 0.0505S6739 | 0.001044725 | 0.00126378 |
| RFXS | 0.032581328 | 0.026591394 | 0.03021749 |
| FOSL1 | 0.031055271 | 0.011035958 | 0.0127338 |
| SOX2 | 0.027714959 | 0.534592984 | 0.5727782 |
| MAX | 0.019835839 | 0.410770498 | 0.4530557 |
| JUN | 0.019178333 | 0.150270397 | 0.16821313 |
| MAFK | 0.010349509 | 0.465275676 | 0.50573443 |
| SRF | 0.004204984 | 0.738275123 | 0.77986809 |
| H3K27ac | 0.002973845 | 0.917903572 | 0.94305161 |
| H3K23ac | 5.85E−04 | 0.964973259 | 0.97801344 |
| H3K9me3 | −2.90E−04 | 0.990659141 | 0.99065914 |
| NRF1 | −0.002638172 | 0.865416367 | 0.90147538 |
| KDMSA | −0.028412481 | 0.005045313 | 0.00600632 |
| H3K79mel | −0.047843621 | 0.006024217 | 0.00705963 |
| REST | −0.051251703 | 2.21E−04 | 2.72E−04 |
| H3K79me2 | −0.094205291 | 4.83E−09 | 7.24E−09 |
| SIX5 | −0.149142911 | 4.39E−20 | 9.98E−20 |
| H3K36me3 | −0.180658975 | 8.81E−10 | 1.3SE−09 |
| ZNF274 | −0.286114609 | 8.29E−62 | 1.24E−60 |

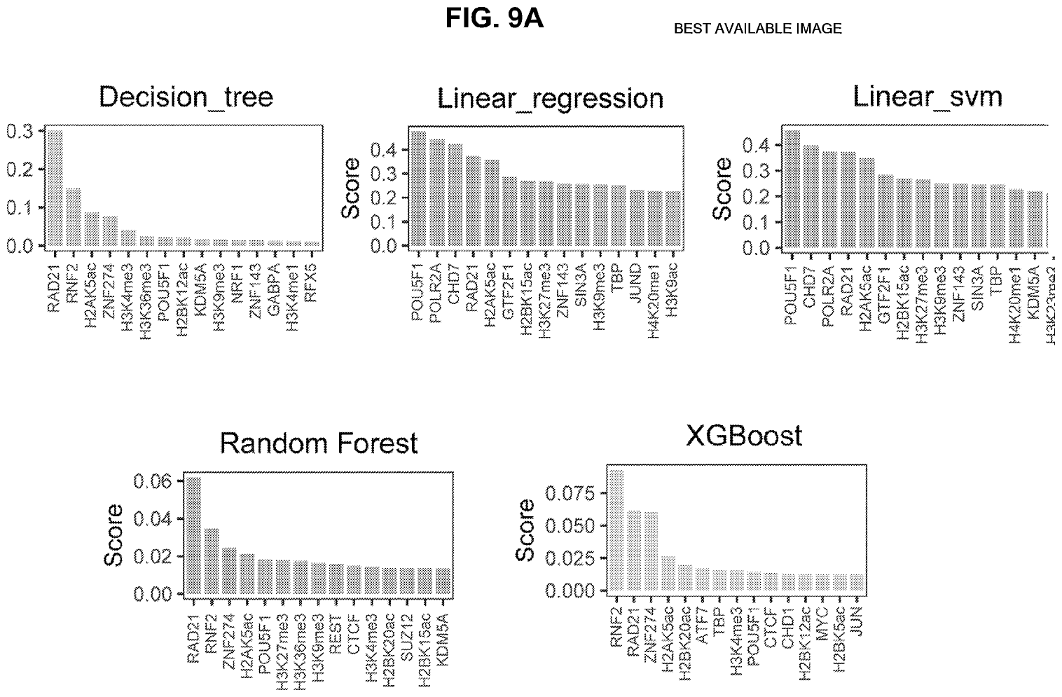

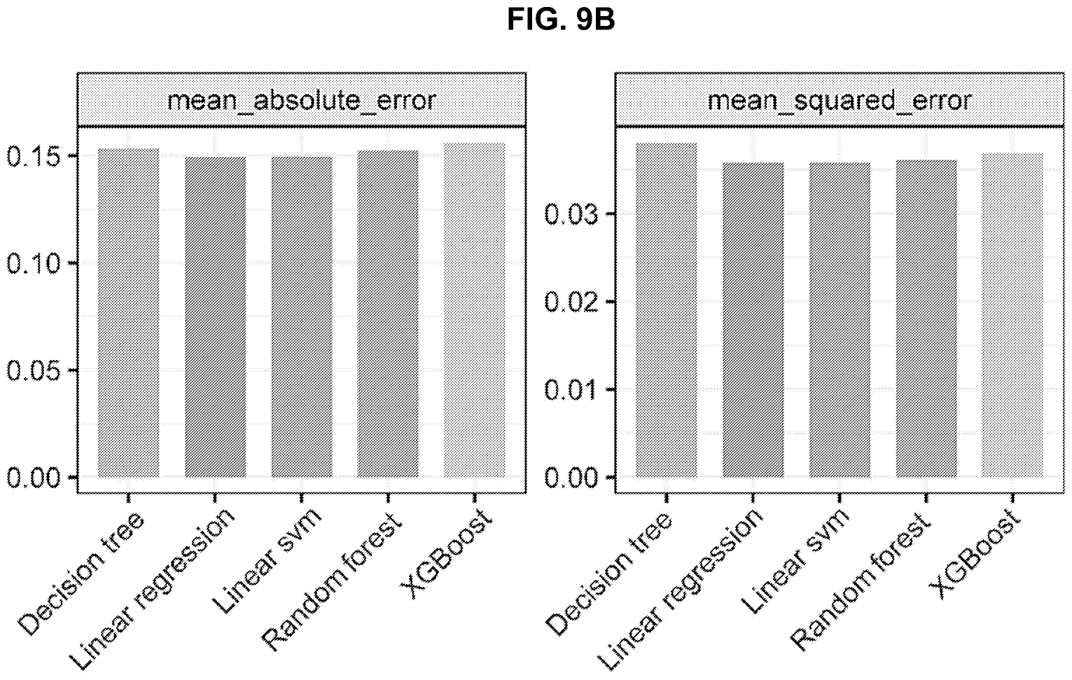

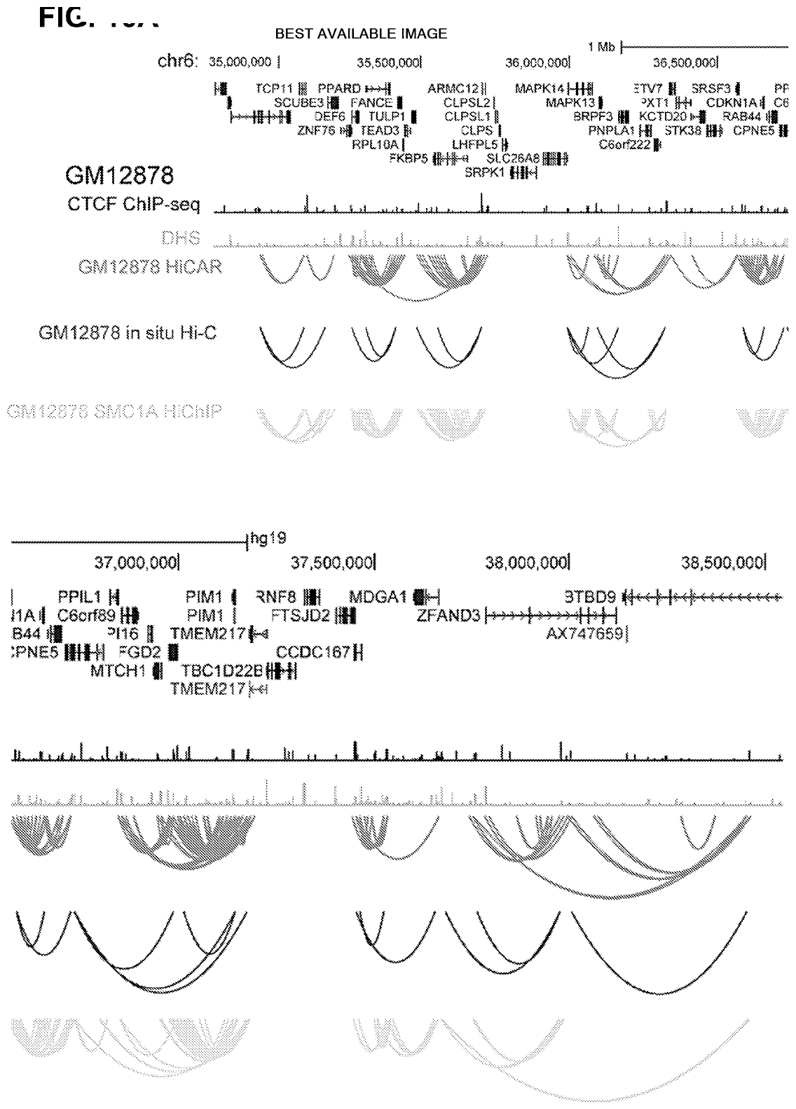

Finally, to gain a more comprehensive view of the epigenome features important for the spatial activity of chromatin. machine learning approaches were used to investigate the contribution of 26 histone modifications and the binding of 49 different TFs on chromatin spatial activity. Five regression methods (Decision tree, Linear regression, XGBoost, Random forest, and Linear-kernel support vector machine (Linear SVM)), were used to define the 15 top-ranked features from each model (FIG. 9A, Table 8, detailed in material and methods, infra).

TABLE 8
The Full List of Top-Ranked Important Features Predicted by Five Regression Models

| Feature | decision_tree | linear_svm | linear_regression | Random Forest | xgboost |
|---|---|---|---|---|---|
| ATF3 | 0.002942469 | 0.015047805 | 0.012685019 | 0.011249957 | 0.008091381 |
| BACH1 | 0 | 0.051291716 | 0.056758944 | 0.011847479 | 0.009191143 |
| BRCA1 | 0.001830542 | 0.069394748 | 0.076600958 | 0.011405115 | 0.009686794 |
| CHD1 | 0.007826727 | 0.027577553 | 0.037526231 | 0.013329972 | 0.013107327 |
| CHD2 | 0.000569795 | 0.158350128 | 0.16592268 | 0.009147057 | 0.009047926 |
| CHD7 | 0.00150294 | 0.398724256 | 0.423846792 | 0.012790449 | 0.009517225 |
| CTBP2 | 0 | 0.024103711 | 0.024654371 | 0.011347687 | 0.00897886 |
| CTCF | 0.004489743 | 0.047691954 | 0.046427483 | 0.014819142 | 0.013572474 |
| EGR1 | 0.001854849 | 0.110107406 | 0.113614783 | 0.011585596 | 0.01000852 |
| EP300 | 0 | 0.089153331 | 0.089297179 | 0.010358923 | 0.009496541 |
| FOSL1 | 0.002336874 | 0.040249773 | 0.037095215 | 0.012479175 | 0.009348956 |
| GABPA | 0.013435892 | 0.007207174 | 0.004135651 | 0.011547407 | 0.011103799 |
| GTF2F1 | 0.010944498 | 0.283237868 | 0.287620047 | 0.012555719 | 0.010912632 |
| H2AK5ac | 0.08672934 | 0.348245609 | 0.358464967 | 0.021290381 | 0.026183333 |
| H2BK120ac | 0 | 0.083948085 | 0.088428885 | 0.009121063 | 0.009023073 |
| H2BK12ac | 0.020961488 | 0.127449842 | 0.124511543 | 0.01304315 | 0.012725298 |
| H2BK15ac | 0.007493292 | 0.269186247 | 0.271084437 | 0.013576386 | 0.011557311 |
| H2BK20ac | 0.008844616 | 0.060034405 | 0.060144684 | 0.013795727 | 0.019588193 |
| H2BK5ac | 0 | 0.027413294 | 0.031612956 | 0.009339499 | 0.012431573 |
| H3K14ac | 0.003849901 | 0.087454602 | 0.086489529 | 0.010374597 | 0.008590821 |
| H3K18ac | 0.004603852 | 0.143751432 | 0.146933019 | 0.009451399 | 0.009114926 |
| H3K23ac | 0.004460048 | 0.070785078 | 0.069151799 | 0.012329926 | 0.010435848 |
| H3K23me2 | 0.011245963 | 0.210764983 | 0.212482272 | 0.012726688 | 0.010006819 |
| H3K27ac | 0.00142827 | 0.076231124 | 0.082445187 | 0.013535529 | 0.009923106 |
| H3K27me3 | 0.006274805 | 0.264723544 | 0.267807634 | 0.018019657 | 0.012383469 |
| H3K36me3 | 0.024663389 | 0.001022123 | 0.002384416 | 0.017448747 | 0.010223091 |
| H3K4ac | 0 | 0.001801705 | 0.000835903 | 0.008959393 | 0.009003838 |
| H3K4me1 | 0.012635185 | 0.191763828 | 0.192187825 | 0.012214466 | 0.009169876 |
| H3K4me2 | 0 | 0.105986392 | 0.104884314 | 0.009645867 | 0.009630124 |
| H3K4me3 | 0.041835436 | 0.181694812 | 0.194663812 | 0.014309336 | 0.015552193 |
| H3K56ac | 0 | 0.162900151 | 0.168035108 | 0.010902655 | 0.010062593 |
| H3K79me1 | 0.009669848 | 0.149842465 | 0.149048456 | 0.013190409 | 0.012395474 |
| H3K79me2 | 0.005545473 | 0.010073119 | 0.010816857 | 0.012947681 | 0.009371148 |
| H3K9ac | 0 | 0.201149376 | 0.226578398 | 0.012075599 | 0.011657927 |
| H3K9me3 | 0.01596266 | 0.251421621 | 0.255226647 | 0.016487303 | 0.010986959 |
| H4K20me1 | 0.005545218 | 0.228135728 | 0.227630164 | 0.012363562 | 0.009681554 |
| H4K5ac | 0.002759048 | 0.180203554 | 0.186740944 | 0.01011836 | 0.009408799 |
| H4K8ac | 0.002552595 | 0.191089956 | 0.192633768 | 0.011010995 | 0.011553083 |
| H4K91ac | 0 | 0.020628333 | 0.022092798 | 0.008231345 | 0.009025132 |
| HDAC2 | 0.005022805 | 0.011527897 | 0.007347331 | 0.010102166 | 0.009322836 |
| JUN | 0.008043633 | 0.071078648 | 0.06864813 | 0.01274435 | 0.012398188 |
| JUND | 0 | 0.183857771 | 0.233081364 | 0.010534497 | 0.008812678 |
| KDM1A | 0.001370451 | 0.072464241 | 0.074238598 | 0.011895036 | 0.010062075 |
| KDM5A | 0.016536759 | 0.221558336 | 0.225727641 | 0.013555517 | 0.011558511 |
| MAFK | 0.004627279 | 0.06930695 | 0.075717942 | 0.012807389 | 0.010093629 |
| MAX | 0.00321807 | 0.088706636 | 0.0913794 | 0.012627411 | 0.009222279 |
| MYC | 0.003291591 | 0.116101025 | 0.115353581 | 0.01236887 | 0.012498749 |
| NANOG | 0 | 0.119833232 | 0.123402403 | 0.010832612 | 0.008885422 |
| NRF1 | 0.01469342 | 0.082631869 | 0.083703047 | 0.013325931 | 0.011514658 |
| OCT4 | 0.021519186 | 0.455499866 | 0.475272254 | 0.018182386 | 0.014170718 |
| PHF8 | 0.002580863 | 0.141208303 | 0.146287608 | 0.010368982 | 0.009759104 |
| POLR2A | 0.002213895 | 0.373687493 | 0.444087977 | 0.010309027 | 0.010616809 |
| POLR2AphosphoS5 | 0.007710254 | 0.098389297 | 0.051214746 | 0.01087548 | 0.009452198 |
| RAD21 | 0.300367752 | 0.372294837 | 0.373670578 | 0.061753321 | 0.061522331 |
| RBBP5 | 0 | 0.048582694 | 0.052429161 | 0.011330924 | 0.011163178 |
| REST | 0.00850239 | 0.165140769 | 0.165640786 | 0.016118805 | 0.011873983 |
| RFX5 | 0.011542399 | 0.064360152 | 0.069789542 | 0.013110082 | 0.009874568 |
| RNF2 | 0.150549073 | 0.119540646 | 0.116568586 | 0.034645792 | 0.092443749 |
| RXRA | 0 | 0.011696898 | 0.012923972 | 0.012392603 | 0.007833352 |
| SAP30 | 0.008100795 | 0.016381032 | 0.010679592 | 0.012144165 | 0.010002629 |
| SIN3A | 0 | 0.246738512 | 0.256234446 | 0.009561917 | 0.010110429 |
| SIX5 | 0 | 0.001949766 | 0.004126885 | 0.013221412 | 0.010245181 |
| SOX2 | 0 | 0.024471163 | 0.032163242 | 0.009834659 | 0.01088312 |
| SP1 | 0.007941009 | 0.210573884 | 0.222407019 | 0.011430816 | 0.010636424 |
| SRF | 0.00252392 | 0.133791899 | 0.138459677 | 0.012163348 | 0.008982535 |
| SUZ12 | 0 | 0.016799504 | 0.015566909 | 0.013649549 | 0.011222387 |
| TAF1 | 0 | 0.019965741 | 0.020553547 | 0.009779353 | 0.010462513 |
| TAF7 | 0 | 0.074676303 | 0.064270351 | 0.0117508 | 0.017236605 |
| TBP | 0.001071115 | 0.246426373 | 0.251080765 | 0.013519716 | 0.01573159 |
| TCF12 | 0.001934404 | 0.03515402 | 0.033478216 | 0.011211322 | 0.009002618 |
| USF1 | 0.001196758 | 0.062093917 | 0.063624406 | 0.012654726 | 0.007804291 |
| USF2 | 0 | 0.191553036 | 0.196479525 | 0.010795582 | 0.012266138 |
| YY1 | 0 | 0.081252529 | 0.083045455 | 0.010270001 | 0.009256243 |
| ZNF143 | 0.014190636 | 0.24817469 | 0.258181011 | 0.012783366 | 0.010932796 |
| ZNF274 | 0.076456787 | 0.171268159 | 0.216114567 | 0.024374695 | 0.060396362 |

The five regression models have similar performance as indicated by comparable mean squared error (MES) and mean absolute error (MAE) (FIG. 9B). To identify the high-confident epigenome features important to chromatin's spatial interactive activity, the positive features, defined as “union features”, were identified by at least two models independently. Using this approach, 22 “union features” were predicted to be important for the spatial activity of chromatin (FIG. 5C). Among these union features, Cohesin (RAD21), CTCF, and ZNF143 are the well-known regulators important for 3D genome organization. Additional features, such as pluripotency factor POU5F1, the PRC1 core component RNF2 (also known as RING1B), histone H3K27me3 modification, and transcription activation marks H3K36me3/H4K20mel/RNA Pol2, with known function in regulating high-order chromatin organization were identified. The identification of multiple union features with previously validated roles in regulating high-order chromatin organization (FIG. 5C, highlighted in blue) indicates that these models were capable of accurately predicting regulators that are important for chromatin interaction activity.

### Example 7

### Identification of Long-Range Cis-Regulatory Chromatin Interactions in GM12878 and Mouse Embryonic Stem Cells (Mescs) with HICAR

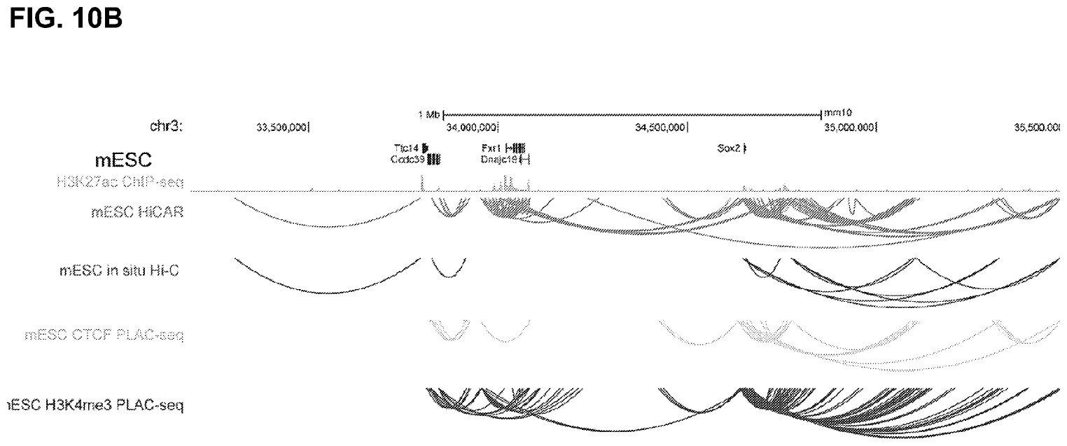

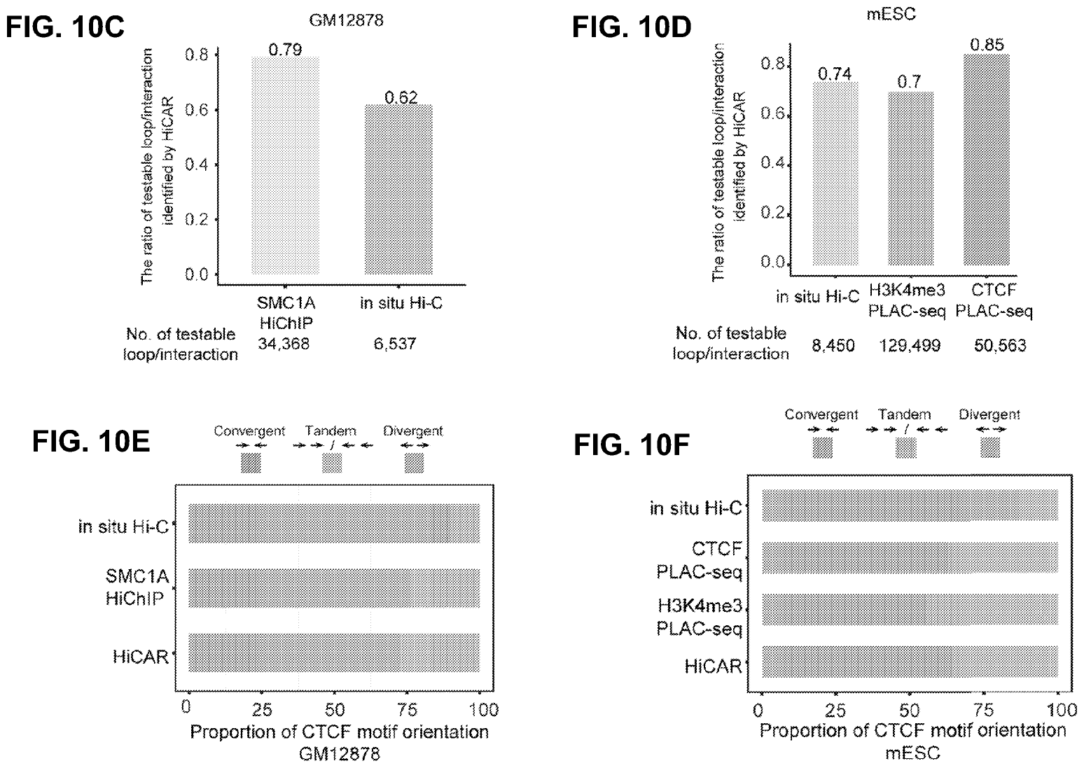

Lastly, to demonstrate the general applicability of HiCAR in other cell types. HiCAR was applied to human lymphoblastoid cell line GM12878 and mouse embryonic stem cells (mESCs). For each cell type, ˜100,000 cells were used as input sample and generated high quality HiCAR DNA libraries (Table 3, supra). Using the same approach described in FIG. 3A-FIG. 3C, then 42,459 and 91,809 significant (MAPS FDR <0.01) high resolution (10 KB bin) interactions in GM12878 and mESCs, respectively, were identified (FIG. 10A and FIG. 108; Tables 9A-9D and Tables 10A-10C for the full list of MAPS interactions and HiCCUPS loops identified in GM12878 and mESCs).

Each of Tables 9A-9D are representative of the data generated in the analysis. Each of Tables 9A-9D represents a “snapshot” of the expansive volume of data generated during an analysis. As disclosed supra, HiCARTools or NF-Core/HiCAR is a bioinformatics best-practice analysis pipeline for processing these data.

Each of Tables 10A-10C are representative of the data generated in the analysis. Each of Tables 10A-10C represents a “snapshot” of the expansive volume of data generated during an analysis. As disclosed supra. HiCARTools or NF-Core/HiCAR is a bioinformatics best-practice analysis pipeline for processing these data.

TABLE 9A
Representative List of HiCCUPPS Loops and MAPS Interactions in mESC Cells Identified in HiCAR Datasets

| | | | | | | | | | | | | | Clus- |
|---|---|---|---|---|---|---|---|---|---|---|---|---|---|
| | | | | | | | | | | Clus- | | | ter |
| | | | | | | | | | Cluster | ter | Cluster | ClusterNeg | Sum- |
| chr1 | start1 | end1 | chr2 | start2 | end2 | count | expected | fdr | Label | Size | Type | Log10P | mit |
| chr1 | 4770000 | 4779999 | chr1 | 4890000 | 4899999 | 33 | 11.07160058 | 1.01E−05 | chr1_01 | 1 | Singleton | 7.612616771 | 1 |
| chr1 | 5100000 | 5109999 | chr1 | 5900000 | 5909999 | 13 | 1.928113341 | 9.13E−06 | chr1_02 | 1 | Singleton | 7.658489517 | 1 |
| chr1 | 7390000 | 7399999 | chr1 | 7670000 | 7679999 | 20 | 4.802209306 | 1.66E−05 | chr1_03 | 1 | Singleton | 7.374521531 | 1 |
| chr1 | 10830000 | 10839999 | chr1 | 11350000 | 11359999 | 22 | 3.334983072 | 1.61E−09 | chr1_04 | 1 | Singleton | 11.74942109 | 1 |
| chr1 | 63850000 | 63859999 | chr1 | 64440000 | 64449999 | 13 | 2.108437415 | 2.38E−05 | chr1_05 | 1 | Singleton | 7.199330197 | 1 |
| chr1 | 64000000 | 64009999 | chr1 | 64440000 | 64449999 | 19 | 3.62633748 | 1.08E−06 | chr1_06 | 1 | Singleton | 8.678048283 | 1 |
| chr1 | 93720000 | 93729999 | chr1 | 93740000 | 93749999 | 92 | 49.92916706 | 1.34E−05 | chr1_07 | 1 | Singleton | 7.4767328 | 1 |
| chr1 | 5940000 | 5949999 | chr1 | 6130000 | 6139999 | 18 | 3.270592906 | 1.19E−06 | chr1_08 | 1 | Singleton | 8.633985463 | 1 |
| chr1 | 60150000 | 60159999 | chr1 | 60860000 | 60869999 | 15 | 2.834715253 | 2.34E−05 | chr1_09 | 1 | Singleton | 7.206981606 | 1 |
| chr1 | 21250000 | 21259999 | chr1 | 22460000 | 22469999 | 13 | 1.823948327 | 5.02E−06 | chr1_ | 1 | Singleton | 7.946121818 | 1 |
| | | | | | | | | | 010 | | | | |
| chr1 | 11150000 | 11159999 | chr1 | 11560000 | 11569999 | 19 | 3.035779536 | 7.01E−08 | chr1_ | 1 | Singleton | 9.97049775 | 1 |
| | | | | | | | | | 011 | | | | |
| chr1 | 11270000 | 11279999 | chr1 | 11560000 | 11569999 | 21 | 5.234687928 | 1.60E−05 | chr1_ | 1 | Singleton | 7.395580581 | 1 |
| | | | | | | | | | 012 | | | | |
| chr1 | 13780000 | 13789999 | chr1 | 14910000 | 14919999 | 12 | 1.746747827 | 2.09E−05 | chr1_ | 1 | Singleton | 7.263381956 | 1 |
| | | | | | | | | | 013 | | | | |
| chr1 | 21460000 | 21469999 | chr1 | 21960000 | 21969999 | 13 | 2.38949941 | 8.78E−05 | chr1_ | 1 | Singleton | 6.565669412 | 1 |
| | | | | | | | | | 014 | | | | |
| chr1 | 13560000 | 13569999 | chr1 | 13580000 | 13589999 | 75 | 37.66889436 | 1.10E−05 | chr1_ | 1 | Singleton | 7.573122492 | 1 |
| | | | | | | | | | 015 | | | | |
| chr1 | 13840000 | 13849999 | chr1 | 14420000 | 14429999 | 15 | 2.536767784 | 6.16E−06 | chr1_ | 1 | Singleton | 7.848564806 | 1 |
| | | | | | | | | | 016 | | | | |
| chr1 | 7390000 | 7399999 | chr1 | 7760000 | 7769999 | 17 | 3.885604054 | 5.68E−05 | chr1_ | 1 | Singleton | 6.776569993 | 1 |
| | | | | | | | | | 017 | | | | |

TABLE 9B
Representative List of HiCCUPPS Loops and MAPS Interactions in mESC Cells Identified in In Situ HiC Datasets

| | | | | | | | | | | | | | expected | | | | | | | | | | |
|---|---|---|---|---|---|---|---|---|---|---|---|---|---|---|---|---|---|---|---|---|---|---|---|
| chr1 | s1 | s2 | chr2 | s1 | s2 | [symbol] | [symbol] | [symbol] | [symbol] | color | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | — | — | — | — | [symbol] | [symbol] | [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

TABLE 9C
Representative List of HiCCUPPS Loops and MAPS Interactions in mESC Cells Identified in PLAC-seq CTCF Datasets

| chr1 | start1 | end1 | chr2 | start2 | end2 | obs | exp | fdr | type | summit |
|---|---|---|---|---|---|---|---|---|---|---|
| chr1 | 4490000 | 4499999 | chr1 | 4650000 | 4659999 | 19 | 5.76778213 | 4.03E−04 | Cluster | 0 |
| chr1 | 4490000 | 4499999 | chr1 | 4660000 | 4669999 | 18 | 6.12016133 | 0.00210786 | Cluster | 0 |
| chr1 | 4490000 | 4499999 | chr1 | 4670000 | 4679999 | 20 | 5.71599266 | 1.19E−04 | Cluster | 1 |
| chr1 | 4490000 | 4499999 | chr1 | 4680000 | 4689999 | 16 | 5.83456939 | 0.00809667 | Cluster | 0 |
| chr1 | 4490000 | 4499999 | chr1 | 4750000 | 4759999 | 18 | 5.00856216 | 2.25E−04 | Cluster | 0 |
| chr1 | 4490000 | 4499999 | chr1 | 4760000 | 4769999 | 24 | 6.76957095 | 2.46E−06 | Cluster | 1 |
| chr1 | 4490000 | 4499999 | chr1 | 5010000 | 5019999 | 19 | 3.453048 | 5.22E−08 | Cluster | 1 |
| chr1 | 4490000 | 4499999 | chr1 | 5020000 | 5029999 | 14 | 2.93493758 | 9.81E−05 | Cluster | 0 |
| chr1 | 4510000 | 4319999 | chr1 | 4750000 | 4759999 | 17 | 3.74155484 | 2.38E−05 | Cluster | 0 |
| chr1 | 4510000 | 4519999 | chr1 | 4760000 | 4769999 | 22 | 5.04084069 | 2.32E−07 | Cluster | 1 |
| chr1 | 4520000 | 4529999 | chr1 | 4760000 | 4769999 | 12 | 3.51272658 | 0.00572678 | Cluster | 0 |
| chr1 | 4530000 | 4539999 | chr1 | 4760000 | 4769999 | 14 | 4.06004989 | 0.00218068 | Cluster | 0 |
| chr1 | 5170000 | 5179999 | chr1 | 5910000 | 5919999 | 12 | 1.77562541 | 1.67E−05 | Singleton | 0 |
| chr1 | 5900000 | 5909999 | chr1 | 6130000 | 6139999 | 13 | 3.64746437 | 0.00259196 | Cluster | 0 |
| chr1 | 5910000 | 5919999 | chr1 | 6120000 | 6129999 | 14 | 2.42944849 | 1.39E−05 | Cluster | 0 |
| chr1 | 5910000 | 5919999 | chr1 | 6130000 | 6139999 | 48 | 5.41655457 | 3.45E−27 | Cluster | 1 |
| chr1 | 5910000 | 5919999 | chr1 | 6140000 | 6149999 | 23 | 4.96266852 | 3.35E−07 | Cluster | 0 |
| chr1 | 5910000 | 5919999 | chr1 | 6150000 | 6159999 | 23 | 4.90009961 | 3.24E−08 | Cluster | 0 |

TABLE 9D
Representative List of HiCCUPPS Loops and MAPS Interactions in mESC Cells Identified in PLAC-seq CTCF Datasets

| chr1 | start1 | end1 | chr2 | start2 | end2 | obs | exp | fdr | type | summit |
|---|---|---|---|---|---|---|---|---|---|---|
| chr1 | 4485000 | 4489999 | chr1 | 5015000 | 5019999 | 12 | 2.88935568 | 8.49E−04 | Cluster | 0 |
| chr1 | 4490000 | 4494999 | chr1 | 4665000 | 4669999 | 32 | 12.6249207 | 2.33E−14 | Cluster | 1 |
| chr1 | 4490000 | 4494999 | chr1 | 4670000 | 4674999 | 33 | 10.8952026 | 3.02E−06 | Cluster | 0 |
| chr1 | 4490000 | 4494999 | chr1 | 4675000 | 4679999 | 29 | 9.97345079 | 3.09E−050 | Cluster | 0 |
| chr1 | 4490000 | 4494999 | chr1 | 4680000 | 4684999 | 21 | 8.40790858 | 0.00360232 | Cluster | 0 |
| chr1 | 4490000 | 4494999 | chr1 | 4685000 | 4689999 | 44 | 12.7893897 | 2.40E−10 | Cluster | 0 |
| chr1 | 4490000 | 4494999 | chr1 | 4690000 | 4694999 | 34 | 11.0342173 | 1.42E−06 | Cluster | 0 |
| chr1 | 4490000 | 4494999 | chr1 | 4725000 | 4729999 | 21 | 7.03089337 | 4.07E−04 | Cluster | 0 |
| chr1 | 4490000 | 4494999 | chr1 | 4740000 | 4744999 | 23 | 8.02463167 | 3.40E−04 | Cluster | 0 |
| chr1 | 4490000 | 4494999 | chr1 | 4745000 | 4749999 | 21 | 6.59420452 | 1.75E−04 | Cluster | 0 |
| chr1 | 4490000 | 4494999 | chr1 | 4750000 | 4754999 | 28 | 11.4473462 | 7.30E−04 | Custer | 0 |
| chr1 | 4490000 | 4494999 | chr1 | 4755000 | 4759999 | 34 | 6.82202226 | 1.25E−11 | Cluster | 0 |
| chr1 | 4490000 | 4494999 | chr1 | 4760000 | 4764999 | 45 | 7.20671691 | 8.76E−19 | Cluster | 0 |
| chr1 | 4490000 | 4494999 | chr1 | 4765000 | 4769999 | 67 | 8.61845068 | 3.63E−33 | Cluster | 1 |
| chr1 | 4490000 | 4494999 | chr1 | 4770000 | 4774999 | 15 | 4.89029895 | 0.00297138 | Cluster | 0 |
| chr1 | 4490000 | 4494999 | chr1 | 4775000 | 4779999 | 27 | 6.30319345 | 5.52E−08 | Cluster | 0 |
| chr1 | 4490000 | 4494999 | chr1 | 4780000 | 4784999 | 29 | 11.4307767 | 1.17E−04 | Cluster | 0 |
| chr1 | 4490000 | 4494999 | chr1 | 5015000 | 5019999 | 38 | 9.48950471 | 8.04E−11 | Caster | 0 |

TABLE 10A
Representative List of HiCCUPPS Loops and MAPS Interactions in GM12878 Cells Identified in HiCAR Datasets

| | | | | | | | | | | | | | Clus- |
|---|---|---|---|---|---|---|---|---|---|---|---|---|---|
| | | | | | | | | | | Clus- | | | ter |
| | | | | | | | | | Cluster | ter | Cluster | ClusterNeg | Sum- |
| chr1 | start1 | end1 | chr2 | start1 | end2 | count | expected | fdr | Label | Size | Type | Log10P | mit |
| chr1 | 940000 | 949999 | chr1 | 1000000 | 1009999 | 27 | 8.624237 | 4.13E−05 | chr1_01 | 1 | Singleton | 6.87849064 | 1 |
| chr1 | 1000000 | 3009999 | chr1 | 1180000 | 1189999 | 14 | 2.19967103 | 6.06E−06 | chr1_02 | 1 | Singleton | 7.82181339 | 1 |
| chr1 | 1330000 | 1339999 | chr1 | 1350000 | 1359999 | 60 | 25.6858056 | 1.11E−06 | chr1_03 | 1 | Singleton | 8.64035869 | 1 |
| chr1 | 8700000 | 8709999 | chr1 | 8720000 | 8729999 | 167 | 102.926883 | 1.22E−06 | chr1_04 | 1 | Singleton | 8.59612267 | 1 |
| chr1 | 9200000 | 9209999 | chr1 | 9220000 | 9229999 | 92 | 51.2355021 | 3.32E−05 | chr1_05 | 1 | Singleton | 6.98845963 | 1 |
| chr1 | 19990000 | 19999999 | chr1 | 20030000 | 20039999 | 43 | 15.1632796 | 6.72E−07 | chr1_06 | 1 | Singleton | 8.87887974 | 1 |
| chr1 | 19230000 | 19239999 | chr1 | 19290000 | 19299999 | 28 | 8.92236729 | 2.62E−05 | chr1_07 | 1 | Singleton | 7.10655259 | 1 |
| chr1 | 6400000 | 6409999 | chr1 | 6460000 | 6469999 | 34 | 9.77857368 | 1.95E−07 | chr1_08 | 1 | Singleton | 9.46525875 | 1 |
| chr1 | 19270000 | 19279999 | chr1 | 19530000 | 19539999 | 27 | 7.02832469 | 9.71E−07 | chr1_09 | 1 | Singleton | 8.7051461 | 1 |
| chr1 | 19490000 | 19499999 | chr1 | 19530000 | 19539999 | 54 | 24.189295 | 1.90E−05 | chr1_010 | 1 | Singleton | 7.26817062 | 1 |
| chr1 | 36560000 | 36569999 | chr1 | 36600000 | 36609999 | 42 | 15.3436973 | 2.39E−06 | chr1_011 | 1 | Singleton | 8.26621728 | 1 |
| chr1 | 3400000 | 3409999 | chr1 | 3530000 | 3539999 | 22 | 4.25418503 | 3.18E−07 | chr1_012 | 1 | Singleton | 9.70711526 | 1 |
| chr1 | 23870000 | 23879999 | chr1 | 23920000 | 23929999 | 62 | 30.9504167 | 8.03E−05 | chr1_013 | 1 | Singleton | 6.5452301 | 1 |
| chr1 | 11960000 | 11969999 | chr1 | 12020000 | 12029999 | 42 | 15.3180632 | 2.29E−06 | chr1_014 | 1 | Singleton | 8.28668505 | 1 |
| chr1 | 2340000 | 2349999 | chr1 | 2510000 | 2319999 | 21 | 5.63981867 | 4.36E−05 | chr1_015 | 1 | Singleton | 6.85035747 | 1 |
| chr1 | 2480000 | 2489999 | chr1 | 2510000 | 2519999 | 71 | 33.2545901 | 1.84E−06 | chr1_016 | 1 | Singleton | 8.39578792 | 1 |
| chr1 | 55610000 | 55619999 | chr1 | 55670000 | 55679999 | 48 | 21.4219991 | 6.70E−05 | chr1_017 | 1 | Singleton | 6.63691262 | 1 |

TABLE 10B
Representative List of HiCCUPPS Loops and MAPS Interactions in mESC Cells Identified in In Situ HiC Datasets

| chr1 | start1 | end1 | chr2 | start2 | end2 | obs | exp | fdr | type | summit |
|---|---|---|---|---|---|---|---|---|---|---|
| chr1 | 900000 | 904999 | chr1 | 910000 | 914999 | 21 | 8.40312464 | 0.00797539 | Cluster | 1 |
| chr1 | 910000 | 914999 | chr1 | 920000 | 924995 | 27 | 12.3531962 | 0.00994538 | Cluster | 0 |
| chr1 | 915000 | 919999 | chr1 | 995000 | 999999 | 17 | 5.30744638 | 2.73E−04 | Cluster | 0 |
| chr1 | 920000 | 924999 | chr1 | 995000 | 999999 | 23 | 5.30562749 | 1.59E−06 | Cluster | 1 |
| chr1 | 925000 | 929999 | chr1 | 995000 | 999999 | 19 | 6.10960897 | 0.00127753 | Cluster | 0 |
| chr1 | 955000 | 959999 | chr1 | 965000 | 969999 | 38 | 14.0302533 | 1.49E−05 | Singleton | 0 |
| chr1 | 1695000 | 1699999 | chr1 | 1835000 | 1839999 | 16 | 4.13002947 | 4.28E−04 | Cluster | 0 |
| chr1 | 1700000 | 1704999 | chr1 | 1835000 | 1839999 | 12 | 3.52610006 | 0.00943763 | Cluster | 0 |
| chr1 | 1710000 | 1714999 | chr1 | 1835000 | 1839999 | 29 | 7.22048082 | 1.06E−08 | Cluster | 1 |
| chr1 | 1715000 | 1719999 | chr1 | 135000 | 1839999 | 17 | 4.90006868 | 8.66E−04 | Cluster | 0 |
| chr1 | 2105000 | 2109999 | chr1 | 2310000 | 2314999 | 13 | 2.83222163 | 5.05E−05 | Cluster | 0 |
| chr1 | 2120000 | 2124999 | chr1 | 2310000 | 2314999 | 18 | 2.42348997 | 2.07E−08 | Cluster | 0 |
| chr1 | 2125000 | 2129999 | chr1 | 2310000 | 2314999 | 27 | 2.82620997 | 1.28E−16 | Cluster | 1 |
| chr1 | 2125000 | 2129999 | chr1 | 2315000 | 2319999 | 16 | 1.88403743 | 2.94E−08 | Cluster | 0 |
| chr1 | 2125000 | 2129999 | chr1 | 2325000 | 2329999 | 14 | 2.47388222 | 2.70E−05 | Cluster | 0 |
| chr1 | 2130000 | 2134999 | chr1 | 2310000 | 2314999 | 15 | 2.1473131 | 9.97E−07 | Cluster | 0 |
| chr1 | 2345000 | 2349999 | chr1 | 2475000 | 2479999 | 21 | 5.66009252 | 4.83E−06 | Cluster | 1 |
| chr1 | 2345000 | 2349999 | chr1 | 2480000 | 2484999 | 15 | 2.92751302 | 3.69E−05 | Cluster | 0 |

TABLE 10C
Representative List of HiCCUPPS Loops and MAPS Interactions in SMC1 Identified in HiChIP Datasets

| chr1 | start1 | end1 | chr2 | start2 | end2 | obs | exp | fdr | type | summit |
|---|---|---|---|---|---|---|---|---|---|---|
| chr1 | 900000 | 904999 | chr1 | 910000 | 914999 | 21 | 8.40312464 | 0.00797539 | Cluster | 1 |
| chr1 | 910000 | 914999 | chr1 | 920000 | 924999 | 27 | 12.3531962 | 0.00994338 | Cluster | 0 |
| chr1 | 915000 | 919999 | chr1 | 995000 | 999999 | 17 | 5.30744638 | 2.73E−04 | Cluster | 0 |
| chr1 | 920000 | 924999 | chr1 | 995000 | 999999 | 23 | 5.30562749 | 1.59E−06 | Cluster | 1 |
| chr1 | 925000 | 929999 | chr1 | 995000 | 999999 | 19 | 6.10960897 | 0.00127753 | Cluster | 0 |
| chr1 | 955000 | 959999 | chr1 | 965000 | 969999 | 38 | 14.0302533 | 1.49E−05 | Singleton | 0 |
| chr1 | 1695000 | 1699999 | chr1 | 1835000 | 1839999 | 16 | 4.13002947 | 4.286-04 | Cluster | 0 |
| chr1 | 1700000 | 1704999 | chr1 | 1835000 | 1839999 | 12 | 3.52610006 | 0.00943763 | Cluster | 0 |
| chr1 | 1710000 | 1714999 | chr1 | 1835000 | 1839999 | 29 | 7.22048082 | 1.06E−08 | Cluster | 0 |
| chr1 | 1715000 | 1719999 | chr1 | 1835000 | 1839999 | 17 | 4.90006868 | 8.66E−04 | Cluster | 0 |
| chr1 | 2105000 | 2109999 | chr1 | 2310000 | 2314999 | 13 | 2.83222163 | 5.05E−05 | Cluster | 0 |
| chr1 | 2120000 | 2124999 | chr1 | 2310000 | 2314999 | 18 | 2.42348997 | 2.07E−08 | Cluster | 0 |
| chr1 | 2125000 | 2129999 | chr1 | 2310000 | 2314999 | 27 | 2.82620997 | 1.28E−16 | Cluster | 1 |
| chr1 | 2125000 | 2129999 | chr1 | 2315000 | 2319999 | 16 | 1.88403743 | 2.94E−08 | Cluster | 0 |
| chr1 | 2125000 | 2129999 | chr1 | 2325000 | 2329999 | 14 | 2.47388222 | 2.70E−05 | Cluster | 0 |
| chr1 | 2130000 | 2134999 | chr1 | 2310000 | 2314999 | 15 | 2.1473131 | 9.97E−07 | Cluster | 0 |
| chr1 | 2345000 | 2349999 | chr1 | 2475000 | 2479999 | 21 | 5.66009252 | 4.83E−06 | Cluster | 1 |
| chr1 | 2345000 | 2349999 | chr1 | 2480000 | 2484999 | 15 | 2.92751302 | 3.69E−05 | Cluster | 0 |

Consistent with the analysis in Hi hESC, the GM12878 and mESC HiCAR interactions showed high sensitivity in detecting the “testable” HiCCUPS loops and MAPS interactions identified by in situ Hi-C, HiChiP, and PLAC-seq in GM12878 and mESCs (FIG. 10C and FIG. 10D). Importantly, 72.4% of GM12878 interactions and 63.7% mESC interactions identified by HiCAR harbored convergent CTCF motifs on their anchor regions. This ratio was comparable to that observed in GM12878 SMC1A HiChiP (75.8%), mESC CTCF PLAC-seq (62.7%), and mESC H3K4me3 PLAC-seq (55.7%). but lower than the ratio detected in HiCCUPS loops identified by in situ Hi-C in GM12878 (89.8%) and in mESC (86.7%) (FIG. 10E and FIG. 10F). These results indicated that the precision of HiCAR interaction called from GM12878 and mESC was comparable to that of PLAC-scq and HiChIP interactions. Successful identification of these high-confident cis-regulatory chromatin interactions in GM12878 and mESCs clearly demonstrated the broadly applicability of HiCAR.

### SUMMARY OF EXAMPLES

As described herein, HiCAR—a novel co-assay was characterized using H1 hESC. HiCAR identified 46,792 significant long-range chromatin interactions anchored on open chromatin regions at 5 KB resolution. By integrating public epigenome datasets generated by the ENCODE, Epigenome Roadmap, and 4DN consortiums using the same H1 hESC line, the data presented herein demonstrated that epigenetically poised, bivalent, and repressed chromatin states can form massive, significant, and long-range chromatin interactions that are comparable to the interactions associated with active chromatin states. Consistent with other H3K27me3 HiChIP and PRC2 ChIA-PET studies, the H3K27me3-anchored HiCAR interactions were enriched for genes that were silenced in pluripotency stem cells but important for tissue and organ development. Importantly, the high-resolution chromatin contact map generated by HiCAR provided the unique opportunity to compare the high-resolution cRE-anchored interactions associated with distinct epigenome modifications and chromatin states. The examples provided herein showed that the cREs with similar chromatin states (“active”, or “inactive”) interacted with each other more frequently, while the interactions between “active” versus “inactive” chromatin states were less frequent. The data indicated the long-range chromatin interaction can play a role in coordinating epigenome modifications of cREs across linearly separated genomic loci.

Another interesting finding revealed by HiCAR was the weak correlation between cRE spatial interaction activity and transcriptional activity, enhancer activity, and chromatin accessibility. By integrating HiCAR data with public epigenome data, 20 histone marks and TF binding interactions that are significantly enriched on cRE-anchored interactions hotspots were identified. Five machine learning approaches to predict 22 “union features” important for the spatial interaction activity of cREs in H1 hESC were also employed. Many of the epigenetic signatures that were enriched on HiCAR interaction hotspots or predicated by machine learning—such as CTCF, Cohesin, ZNF143, POU5F1, RNF2, H3K27me3, H3K4mel—as well as active transcription marks including H3K36me3, H4K20mel, RNA Pol2) were known regulators of 3D genome structure.

With HiCAR data, 2,096 open chromatin-anchored interaction hotspots in H1 hESCs were identified. In previous studies, other groups carried out similar analyses with in situ Hi-C and PLAC-seq data, and discovered frequently interacting regions (FIREs) and super-interactive promoters (SIPs) in the human genome. Like FIREs and SIPs, HiCAR interaction hotspots exhibited unusually high chromatin interaction activity compared to other genomic loci. Notably, FIREs are enriched for super-enhancers and are near genes that are tissue-specifically expressed in 21 primary human tissues and cell types. HiCAR interaction hotspots, however, are not enriched for the super-enhancer mark H3K27ac. The GO enrichment analysis found that GO terms overrepresented on HiCAR interaction hotspots predominantly related to cell proliferation, chromatin organization, as well as neuronal, cardiovascular, blood vessel, and skeletal system differentiation. (Table 6B). There was no pluripotency genes or pluripotency related GO terms enriched on HiCAR interaction hotspots. In contrast, SIPs were enriched for lineage-specific genes in human brain cells. These differences between HiCAR interaction hotspots, FIREs, and SIPs can be due to two potential phenomena. First, the genome organization of hESCs is intrinsically different from that of terminally differentiated cells found in human adult tissues. Or, second, in situ Hi-C, PLAC-seq, and HiCAR each capture a subset of the “true” interactions present in the 3D genome. Therefore, FIREs (by Iii-C), SIPs (by H3K4me3 PLAC-seq), and HiCAR interaction hotspots may represent the top ranked interaction hotspots or hubs that are sampled from different types of chromatin interactions.

Most importantly, these data demonstrated that HiCAR is a robust, sensitive, and cost-effective method that can be used to simultaneously study genome architecture, chromatin accessibility, and the transcriptome from the same low-input samples. Compared to existing methods, the technical advantages of HiCAR are multifold. First. HiCAR required substantially less sequencing depth than in situ Hi-C to identify high-resolution, significant, long-range chromatin interactions anchored on cREs. Second, compared with HiChIP and PLAC-seq, HiCAR did not rely on ChIP-grade antibody-mediated immunoprecipitation to pull down chromatin interactions bound by a specific protein or histone modification. Thus, HiCAR enabled comprehensive analysis of open chromatin-anchored interactions associated with an array of diverse histone mark, TF binding, and chromatin states. Third, compared to state-of-the-art methods such as Trac-looping, with similar sequencing depth, HiCAR generated ˜17-fold more informative long-range cis-PETs despite starting from 1,000-fold lower input cell number. Fourth, by applying HiCAR in GM12878 and mESCs, HiCAR proved itself to be a sensitive and robust assay which is broadly applicable in multiple cell types with low input samples.

Taken together, the data presented herein demonstrate the technical advancement and general applicability of HiCAR, which can be used for multimodal analysis of low-input materials.

1.-9. (canceled)

10. A method of performing a multi-omics assay in a single population of cells, the method comprising:
i. identifying cis-regulatory chromatin interactions and characterizing chromatin accessibility by purifying and tagmenting DNA and performing PCR using the purified and tagmented DNA to generate a DNA library; and
ii. analyzing the transcriptome by collecting cytoplasmic and nucleic RNA while performing step (i) and creating an RNA-Seq library using the collected RNA.

11. The method of claim 10, wherein purifying and tagmenting DNA comprises one or more of the following:
isolating nuclei from a population of cells;
incubating the isolated nuclei with an assembled Tn5 transposome;
digesting the isolated nuclei with a first restriction enzyme;
incubating the digested nuclei with a splint oligonucleotide;
ligating in situ the Tn5 adaptors to the proximal genomic DNA;
reversing the crosslink;
purifying the reverse cross-linked DNA and dissolving the purified DNA;
digesting the purified DNA with a second restriction enzyme;
circularizing the digested DNA and purifying the circularized DNA;
digesting the purified DNA with a third restriction enzyme, or any combination thereof.

12. The method of claim 10, wherein analyzing the transcriptome comprises one or more of the following:
combining supernatant comprising cytoplasmic RNA and supernatant comprising nucleic RNA;
reversing the crosslink;
purifying the reverse crosslinked RNA;
dissolving the purified RNA;
treating the purified RNA with DNase;
creating an RNA-Seq library,
or any combination thereof.

13. The method of claim 10, further comprising processing the resulting DNA library, wherein processing the resulting DNA library comprises mapping and visualizing the uniquely mapped paired-end tags using a bioinformatics software program for visualizing molecular interactions, generating a comprehensive map of cis-regulatory chromatin contacts, calculating a cumulative interactive score for each anchor interaction anchor, or any combination thereof.

14.-19. (canceled)

20. The method of claim 11, wherein the first restriction enzyme is CviQI, the second restriction enzyme is NIaIII, and the third restriction enzyme is PmeI.

21. The method of claim 1, wherein the population of cells is cross-linked prior to the isolating nuclei step (i).

22. The method of claim 11, wherein the isolating nuclei step further comprises centrifuging the cells to isolate the nuclei and collecting the supernatant comprising cytoplasmic RNA.

23. The method of claim 11, wherein incubating the isolated nuclei step further comprises centrifuging the isolated nuclei and collecting the supernatant comprising the nucleic RNA.

24. The method of claim 11, further comprising assembling the Tn5 transposome.

25. The method of claim 24, wherein assembling the Tn5 transposome comprises annealing two Tn5 adaptors and incubating the annealed Tn5 adaptors with a Tn5 transposase.

26.-27. (canceled)

28. The method of claim 1, wherein the performing PCR step comprises mixing the digested purified DNA with dNTPs, a forward primer, a reverse primer, and a polymerase.

29. (canceled)

30. The method of claim 2, wherein the resulting amplified DNA fragments contain one end derived from the CviQI digested genomic DNA and one end derived from the Tn5-tagmented open chromatin sequence.

31. The method of claim 30, wherein the end derived from the CviQI digested genomic DNA is captured by Read 1 of each pair-end sequence and the end derived from the Tn5-tagmented open chromatin sequence is captured by Read 2 of each pair-end sequence.

32. The method of claim 2, further comprising using gel extraction to obtain those PCR products having a size of about 400-600 bp, and subjecting the gel extracted PCR products to deep sequencing.

33. (canceled)

34. The method of claim 12, wherein creating an RNA-Seq library comprises combining supernatant comprising cytoplasmic RNA and supernatant comprising nucleic RNA, reversing the crosslink, purifying the reverse crosslinked RNA, dissolving the purified RNA, treating the purified RNA with DNase, and creating an RNA-Seq library.

35. (canceled)

36. The method of claim 10, wherein the method does not comprise antibody-mediated immunoprecipitation, adaptor ligation, or biotin pull down.

37. (canceled)

38. The method of claim 11, wherein the population of cells comprise cells obtained from a biosample and then subjected to a crosslinking protocol.

39. The method of claim 38, wherein the biosample is obtained from a subject diagnosed with or is suspected of having a disease or disorder.

40. (canceled)

41. The method of claim 10, further comprising repeating the method using a second population of cells.

42.-46. (canceled)

47. A kit, comprising: one or more components and/or reagents for use in the method of of claim 10.

48.-51. (canceled)
